SEQUENCE LISTING (189 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 227 aa, Mus musculus
MGPLHQFLLLLITALSQALNTTVLQGMAGQSLRVSCTYDALKHWGRRKAWCRQLGEEGPCQRVVSTHGVWLLAFLKKRNGSTVIADDTLAGTVTITLKNLQAGDAGLYQCQSLRGREAEVLQKVLVEVLEDPLDDQDAGDLWVPEESSSFEGAQVEHSTSRNQETSFPPTSILLLLACVLLSKFLAASILWAVARGRQKPGTPVVRGLDCGQDAGHQLQILTGPGGT

SEQ ID NO: 3 - protein, 228 aa, Rattus norvegicus
MEPLHVFVLLLVTELSQALNTTVLQGVAGQSLRVSCTYDALRHWGRRKAWCRQLAEEGPCQRVVSTHGVWLLAFLRKQNGSTVITDDTLAGTVTITLRNLQAGDAGLYQCQSLRGREAEVLQKVVVEVLEDPLDDQDAGDLWVPEESESFEGAQVEHSTSSQVSSCGSPLTYHLPPKEPIRKDLLPTHFHSSPPGLCPPEQASYSQHPLGCGQGQAEAGDTCGQWARL

SEQ ID NO: 4 - protein, 260 aa, Macaca mulatta
MPDPLFSAVQGKDKILHKALCICPWPGKGGMEPLRLLILLFATELSGAHNTTVFQGVEGQSLQVSCPYDSMKHWGRRKAWCRQLGEKGPCQRVVSTHNLWLLSFLRRRNGSTAITDDTLGGTLTITLRNLQPHDAGFYQCQSLHGSEADTLRKVLVEVLADPLDHRDAGDLWVPGESESFEDAHVEHSISRSLLEGEIPFPPTSVLLLLACIFLIKILAASALWAAAWHGQKPGTHPPSEPDCGHDPGHQLQTLPGLRDT

SEQ ID NO: 5 - protein, 230 aa, Macaca fascicularis
MEPLRLLILLFATELSGAHNTTVFQGVEGQSLQVSCPYDSMKHWGRRKAWCRQLGEKGPCQRVVSTHNLWLLSFLRRRNGSTAITDDTLGGTLTITLRNLQPHDAGFYQCQSLHGSEADTLRKVLVEVLADPLDHRDAGDLWVPGESESFEDAHVEHSISRSLLEGEIPFPPTSVLLLLACIFLIKILAASALWAAAWHGQKPGTHPPSEPDCGHDPGHQLQTLPGLRDT

SEQ ID NO: 6 - protein, 230 aa, Equus caballus
MEPLPLLILLSVAELSRGHNTTVFQGTAGRSLKVSCPYNSLMHWGRRKAWCRQLGEDGPCQQVVSTHSLWLLSFLKRRNGSTVITDDALGGILTITLRNLQAHDAGFYQCQSLHGGEADTLRKVLVEVLADPLDHQEPGDLWIPKESESFEDAQVEHSISRSLVEEEIPSLPTSILLLLACIFLSKLLAASAIWAAAWHGQKQETPPASEPDRGHDPGYQLHTLTGERDT

SEQ ID NO: 7 - protein, 233 aa, Sus scrofa
METLGLLLLLWVAELSRAHNTSVFQGTAGQSLRVSCSYNSLKHWGRRKAWCRQLSEEGLCQHVVSTHPTWLLSFLKRRNGSTAITDDALGGTLTITLRNLQAHDAGLYQCQSLHGSEADTLKKVLVEVLADPLESQSKSFQDVQMEHSISRNLSEESLFPPTSTLFLLACVFLSKLLVASALWAAAWHGHKQRTSPAGGLDCGRDPGDQDQTLTDELGESSDQDQTLTELRDT

SEQ ID NO: 8 - protein, 230 aa, Canis lupus familiaris
MEPLWLLILLAVTELSGAHNTTVFQGMAGRSLQVSCPYNSLKHWGRRKAWCRQVDKEGPCQRVVSTHRSWLLSFLKRWNGSTAIVDDALGGTLTITLRNLQAHDAGLYQCQSLYGDEADTLRKVLVEVLADPLDHLDPGDLWIPEESKGFEDAHVEPSVSRSLSEEEIPFPPTSILFLLACIFLSKFLAASALWAAAWRGQKLGTPQASELDCSCDPGYQLQTLTEPRDM

SEQ ID NO: 9 - protein, 26 aa, Artificial Sequence
  Synthetic Construct
QVQLVQSGAEVKKPGSSVKVSCKASG

SEQ ID NO: 10 - protein, 26 aa, Artificial Sequence
  Synthetic Construct
EVQLVQSGAEVKKPGSSVKVSCKASG

SEQ ID NO: 11 - protein, 26 aa, Artificial Sequence
  Synthetic Construct
QVQLVQSGAEVKKPGASVKVSCKASG

SEQ ID NO: 12 - protein, 14 aa, Artificial Sequence
  Synthetic Construct
WVRQAPGQGLEWMG

SEQ ID NO: 13 - protein, 14 aa, Artificial Sequence
  Synthetic Construct
WVRQAPGQRLEWIG

SEQ ID NO: 14 - protein, 30 aa, Artificial Sequence
  Synthetic Construct
RVTITADESTSTAYMELSSLRSEDTAVYYC

SEQ ID NO: 15 - protein, 30 aa, Artificial Sequence
  Synthetic Construct
RVTITADTSASTAYMELSSLRSEDTAVYYC

SEQ ID NO: 16 - protein, 11 aa, Artificial Sequence
  Synthetic Construct
WGQGTLVTVSS

SEQ ID NO: 17 - protein, 23 aa, Artificial Sequence
  Synthetic Construct
DVVMTQTPLSLSVTPGQPASISC

SEQ ID NO: 18 - protein, 23 aa, Artificial Sequence
  Synthetic Construct
GVVMTQTPLSLSVTPGQPASISC

SEQ ID NO: 19 - protein, 23 aa, Artificial Sequence
  Synthetic Construct
GVVMAQTPLSLSVTPGQPASISC

SEQ ID NO: 20 - protein, 23 aa, Artificial Sequence
  Synthetic Construct
DVVMTQSPDSLAVSLGERATINC

SEQ ID NO: 21 - protein, 15 aa, Artificial Sequence
  Synthetic Construct
WYLQKPGQSPQLLIY

SEQ ID NO: 22 - protein, 15 aa, Artificial Sequence
  Synthetic Construct
WYQQKPGQSPKLLIY

SEQ ID NO: 23 - protein, 32 aa, Artificial Sequence
  Synthetic Construct
GVPDRFSGSGSGTDFTLKISRVEAEDVGVYYC

SEQ ID NO: 24 - protein, 32 aa, Artificial Sequence
  Synthetic Construct
GVPDRFSGSGSGTDFTLTISSLQAEDVAVYYC

SEQ ID NO: 25 - protein, 10 aa, Artificial Sequence
  Synthetic Construct
FGQGTKLEIK

SEQ ID NO: 26 - protein, 10 aa, Artificial Sequence
  Synthetic Construct
FGGGTKVEIK

SEQ ID NO: 27 - protein, 123 aa, Artificial Sequence
  Synthetic Construct
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSS

SEQ ID NO: 28 - protein, 123 aa, Artificial Sequence
  Synthetic Construct
QVQLVQSGAEVKKPGSSVKVSCKASGYAFSSDWMNWVRQAPGQGLEWMGRIYPGEGDTNYARKFHGRVTITADESTSTAYMELSSLRSEDTAVYYCARLLRNKPGESYAMDYWGQGTLVTVSS

SEQ ID NO: 29 - protein, 112 aa, Artificial Sequence
  Synthetic Construct
DVVMTQTPLSLSVTPGQPASISCRTSQSLVHSNAYTYLHWYLQKPGQSPQLLIYKVSNRVSGVPDRFSGSGSGTDFTLKISRVEAEDVGVYYCSQSTRVPYTFGQGTKLEIK

SEQ ID NO: 30 - protein, 112 aa, Artificial Sequence
  Synthetic Construct
DVVMTQSPDSLAVSLGERATINCRSSQSLVHSNRYTYLHWYQQKPGQSPKLLIYKVSNRFSGVPDRFSGSGSGTDFTLKISRVEAEDVGVYYCSQSTRVPYTFGQGTKLEIK

SEQ ID NO: 31 - protein, 16 aa, Artificial Sequence
  Synthetic Construct
ARLLRNQPGESYAMDY

SEQ ID NO: 32 - protein, 9 aa, Artificial Sequence
  Synthetic Construct
SQSTRVPYT

SEQ ID NO: 33 - protein, 7 aa, Artificial Sequence
  Synthetic Construct
KVSNRFS

SEQ ID NO: 34 - protein, 9 aa, Artificial Sequence
  Synthetic Construct
YAFSSQWMN

SEQ ID NO: 35 - protein, 17 aa, Artificial Sequence
  Synthetic Construct
RIYPGGGDTNYAGKFQG

SEQ ID NO: 36 - protein, 9 aa, Artificial Sequence
  Synthetic Construct
YAFSSDWMN

SEQ ID NO: 37 - protein, 17 aa, Artificial Sequence
  Synthetic Construct
RIYPGEGDTNYARKFHG

SEQ ID NO: 38 - protein, 16 aa, Artificial Sequence
  Synthetic Construct
ARLLRNKPGESYAMDY

SEQ ID NO: 39 - protein, 16 aa, Artificial Sequence
  Synthetic Construct
RTSQSLVHSNAYTYLH

SEQ ID NO: 40 - protein, 7 aa, Artificial Sequence
  Synthetic Construct
KVSNRVS

SEQ ID NO: 41 - protein, 16 aa, Artificial Sequence
  Synthetic Construct
RSSQSLVHSNRYTYLH

SEQ ID NO: 42 - protein, 110 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCP

SEQ ID NO: 43 - protein, 453 aa, Artificial Sequence
  Synthetic Construct
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 44 - protein, 452 aa, Artificial Sequence
  Synthetic Construct
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 45 - protein, 453 aa, Artificial Sequence
  Synthetic Construct
QVQLVQSGAEVKKPGSSVKVSCKASGYAFSSDWMNWVRQAPGQGLEWMGRIYPGEGDTNYARKFHGRVTITADESTSTAYMELSSLRSEDTAVYYCARLLRNKPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 452 aa, Artificial Sequence
  Synthetic Construct
QVQLVQSGAEVKKPGSSVKVSCKASGYAFSSDWMNWVRQAPGQGLEWMGRIYPGEGDTNYARKFHGRVTITADESTSTAYMELSSLRSEDTAVYYCARLLRNKPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 219 aa, Artificial Sequence
  Synthetic Construct
DVVMTQSPDSLAVSLGERATINCRSSQSLVHSNRYTYLHWYQQKPGQSPKLLIYKVSNRFSGVPDRFSGSGSGTDFTLKISRVEAEDVGVYYCSQSTRVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 219 aa, Artificial Sequence
  Synthetic Construct
DVVMTQTPLSLSVTPGQPASISCRTSQSLVHSNAYTYLHWYLQKPGQSPQLLIYKVSNRVSGVPDRFSGSGSGTDFTLKISRVEAEDVGVYYCSQSTRVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 19 aa, Artificial Sequence
  Synthetic Construct
  VARIANT 1: Xaa = Asp or Glu
  VARIANT 2, 3: Xaa = Any Amino Acid, and each can be present or absent
  VARIANT 5, 6: Xaa = Any Amino Acid
  VARIANT 7: Xaa = Leu or Ile
  VARIANT 8, 9, 10, 11, 12, 13, 14, 15: Xaa = Any Amino Acid, and up to two can be present or absent
  VARIANT 17, 18: Xaa = Any Amino Acid
  VARIANT 19: Xaa = Leu or Ile
XXXYXXXXXXXXXXXYXXX

SEQ ID NO: 50 - protein, 112 aa, Artificial Sequence
  Synthetic Construct
DVZMTQTPLTLSVTIGQPASISCKSSQSLLYSNGKTFLSWLLQRPGQSPKRLIYLVSKLDSGVPDRFAGSGSGTDFTLKISRLEADDLGIYYCMQGTHFPLTFGAGTKLELK

SEQ ID NO: 51 - protein, 116 aa, Artificial Sequence
  Synthetic Construct
EVKLEESGGGLVQPGGSMKLSCAASGFTFSDAWMGWVRQSPEKGLEWVAEIRDKVKNHATYYAESVKGRFTISRDDSKSTVYLQMNTLRADDTGIYYCRLGVFDYWGQGTTLTVSS

SEQ ID NO: 52 - protein, 11 aa, Artificial Sequence
  Synthetic Construct
GYSITSDYAWN

SEQ ID NO: 53 - protein, 16 aa, Artificial Sequence
  Synthetic Construct
YINYSGRTIYNPSLKS

SEQ ID NO: 54 - protein, 11 aa, Artificial Sequence
  Synthetic Construct
ARWNGNYGFAY

SEQ ID NO: 55 - protein, 16 aa, Artificial Sequence
  Synthetic Construct
RSSQSLVHINGNTYLH

SEQ ID NO: 56 - protein, 7 aa, Artificial Sequence
  Synthetic Construct
KVSNRFS

SEQ ID NO: 57 - protein, 9 aa, Artificial Sequence
  Synthetic Construct
SQTTHALFT

SEQ ID NO: 58 - protein, 118 aa, Artificial Sequence
  Synthetic Construct
DVQLQESGPGLVKPSQSLSLTCTVTGYSITSDYAWNWIRQFPGNKLEWMGYINYSGRTIYNPSLKSRISITRDTSKNHFFLQLISVTTEDTATYYCARWNGNYGFAYWGQGTLVTVSA

SEQ ID NO: 59 - protein, 111 aa, Artificial Sequence
  Synthetic Construct
DWMTQNPLSLPVSLGDQASISCRSSQSLVHINGNTYLHWYLQKPGQSPKLLIYKVSNRFSGVPDRFSGSGSGTDFTLKISRVEAEDLGVYFCSQTTHALFTFGSGTKLEIK

SEQ ID NO: 60 - protein, 7 aa, Artificial Sequence
  Synthetic Construct
GYTFTSY

SEQ ID NO: 61 - protein, 15 aa, Artificial Sequence
  Synthetic Construct
IGRSDPTTGGTNYNE

SEQ ID NO: 62 - protein, 9 aa, Artificial Sequence
  Synthetic Construct
VRTSGTGDY

SEQ ID NO: 63 - protein, 16 aa, Artificial Sequence
  Synthetic Construct
RSSQSLVHNNGNTFLH

SEQ ID NO: 64 - protein, 6 aa, Artificial Sequence
  Synthetic Construct
VSNRFS

SEQ ID NO: 65 - protein, 9 aa, Artificial Sequence
  Synthetic Construct
SQTTHVPPT

SEQ ID NO: 66 - protein, 140 aa, Artificial Sequence
  Synthetic Construct
QVQLQQPGAELVKPGASVKLSCKASGYTFTSYWMHWVKQSPGRGLEWIGRSDPTTGGTNYNEKFKTKATLTVDKPSSTAYMQLSSLTSDDSAVYYCVRTSGTGDYWGQGTSLTVSSAKTTAPSVYPLAPVCGGTTGSSVT

SEQ ID NO: 67 - protein, 140 aa, Artificial Sequence
  Synthetic Construct
DVVMTQTPLSLPVSLGDQASISCRSSQSLVHNNGNTFLHWYLQKPGQSPKLLIYKVSNRFSGVPDRFSGSGSGTDFTLKISRVEAEDLGVYFCSQTTHVPPTFGGGTKLEIKRADAAPTVSIFPPSSEQLTSGGASVVCF

SEQ ID NO: 68 - protein, 8 aa, Artificial Sequence
  Synthetic Construct
GFTFTDFY

SEQ ID NO: 69 - protein, 10 aa, Artificial Sequence
  Synthetic Construct
IRNKANGYTT

SEQ ID NO: 70 - protein, 15 aa, Artificial Sequence
  Synthetic Construct
ARIGINNGGSLDYWG

SEQ ID NO: 71 - protein, 12 aa, Artificial Sequence
  Synthetic Construct
QSLLYSENNQDY

SEQ ID NO: 72 - protein, 3 aa, Artificial Sequence
  Synthetic Construct
GAS

SEQ ID NO: 73 - protein, 9 aa, Artificial Sequence
  Synthetic Construct
EQTYSYPYT

SEQ ID NO: 74 - protein, 121 aa, Artificial Sequence
  Synthetic Construct
EVKLLESGGGLVQPGGSMRLSCAASGFTFTDFYMNWIRQPAGKAPEWLGLIRNKANGYTTEYNPSVKGRFTISRDNTQNMLYLQMNTLREDTATYYCARIGINNGGSLDYWGQGVMVTVSS

SEQ ID NO: 75 - protein, 113 aa, Artificial Sequence
  Synthetic Construct
DILINQSPASLTVSAGEKVTMSCKSSQSLLYSENNQDYLAWYQQKPGQFPKLLIYGASNRHTGVPDRFTGSGSGTDFTLTISSVQAEDLADYYCEQTYSYPYTFGAGTKLELK

SEQ ID NO: 76 - protein, 8 aa, Artificial Sequence
  Synthetic Construct
GFTFTDFY

SEQ ID NO: 77 - protein, 10 aa, Artificial Sequence
  Synthetic Construct
IRNKTKGYTT

SEQ ID NO: 78 - protein, 15 aa, Artificial Sequence
  Synthetic Construct
ARIGVNNGGSLDYWG

SEQ ID NO: 79 - protein, 12 aa, Artificial Sequence
  Synthetic Construct
QSLLYSENNQDY

SEQ ID NO: 80 - protein, 3 aa, Artificial Sequence
  Synthetic Construct
GAS

SEQ ID NO: 81 - protein, 9 aa, Artificial Sequence
  Synthetic Construct
EQTYSYPYT

SEQ ID NO: 82 - protein, 122 aa, Artificial Sequence
  Synthetic Construct
EVKLLEFGGGLVQPGGSMRLSCAASGFTFTDFYMNWIRQPAGRAPEWLGLIRNKTKGYTTEYNRSVKGRFTISRDNTQNMLYLQMNSLRPEDTATYYCARIGVNNGGSLDYWGQGVMVTVSS

SEQ ID NO: 83 - protein, 113 aa, Artificial Sequence
  Synthetic Construct
DILIIQSPASLTVSAGARVTMSCKSSQSLLYSENNQDYLAWYQQKPGQFPKLLIYGASNRHTGVPDRFTGSGSGTDFTLTISSVQAEDLADYYCEQTYSYPYTFGAGTKLELK

SEQ ID NO: 84 - protein, 8 aa, Artificial Sequence
  Synthetic Construct
GFTFTDFY

SEQ ID NO: 85 - protein, 10 aa, Artificial Sequence
  Synthetic Construct
IRNKANGYTT

SEQ ID NO: 86 - protein, 15 aa, Artificial Sequence
  Synthetic Construct
ARIGINNGGSLDYWG

SEQ ID NO: 87 - protein, 12 aa, Artificial Sequence
  Synthetic Construct
QSLLYSEKNQDY

SEQ ID NO: 88 - protein, 3 aa, Artificial Sequence
  Synthetic Construct
GAS

SEQ ID NO: 89 - protein, 9 aa, Artificial Sequence
  Synthetic Construct
EQTYSYPYT

SEQ ID NO: 90 - protein, 122 aa, Artificial Sequence
  Synthetic Construct
EVKLLESGGGLVQPGGSMRLSCAASGFTFTDFYMNWIRQPAGKAPEWLGLIRNKANGYTTVYNPSVKGRFTISRDNTQNMLYLQMNTLRGEDTATYYCARIGINNGGSLDYWGQGVMVTVSS

SEQ ID NO: 91 - protein, 113 aa, Artificial Sequence
  Synthetic Construct
DILINQSPASLTVSTGEKVTMSCRSSQSLLYSEKNQDYLAWYQQKPGQFPKLLIYGASYRHTGVPDRFTGSGSGTDFTLTISSVQAEDLADYYCEQTYSYPYTFGAGTKLELK

SEQ ID NO: 92 - protein, 8 aa, Artificial Sequence
  Synthetic Construct
GFTFTDFY

SEQ ID NO: 93 - protein, 10 aa, Artificial Sequence
  Synthetic Construct
IRNKANGYTT

SEQ ID NO: 94 - protein, 15 aa, Artificial Sequence
  Synthetic Construct
ARIGINNGGSLDYWG

SEQ ID NO: 95 - protein, 12 aa, Artificial Sequence
  Synthetic Construct
QSLLYSEKNQDY

SEQ ID NO: 96 - protein, 3 aa, Artificial Sequence
  Synthetic Construct
GAS

SEQ ID NO: 97 - protein, 9 aa, Artificial Sequence
  Synthetic Construct
EQTYSYPYT

SEQ ID NO: 98 - protein, 122 aa, Artificial Sequence
  Synthetic Construct
EVKLLESGGGLVQPGGSMRLSCAASGFTFTDFYMNWIRQPAGKAPEWLGLIRNKANGYTTQYNPSVKGRFTISRDNTQNMLYLQMNTLRGEDTATYYCARIGINNGGSLDYWGQGVMVTVSS

SEQ ID NO: 99 - protein, 113 aa, Artificial Sequence
  Synthetic Construct
DILINQSPASLTVSAGEKVTMSCKSSQSLLYSEKNQDYLAWYQQKPGQSPKLLMYGASYRHTGVPDRFTGSGSGTDFTLTISSVQAEDLADYYCEQTYSYPYTFGAGTKLELK

SEQ ID NO: 100 - protein, 8 aa, Artificial Sequence
  Synthetic Construct
GFTFTDFY

SEQ ID NO: 101 - protein, 10 aa, Artificial Sequence
  Synthetic Construct
IRNKTKGYTT

SEQ ID NO: 102 - protein, 15 aa, Artificial Sequence
  Synthetic Construct
ARIGVNNGGSLDYWG

SEQ ID NO: 103 - protein, 12 aa, Artificial Sequence
  Synthetic Construct
QSLLYSENNQDY

SEQ ID NO: 104 - protein, 3 aa, Artificial Sequence
  Synthetic Construct
GAS

SEQ ID NO: 105 - protein, 9 aa, Artificial Sequence
  Synthetic Construct
EQTYSYPYT

SEQ ID NO: 106 - protein, 122 aa, Artificial Sequence
  Synthetic Construct
EVKLLESGGGLVQPGGSMRLSCAASGFTFTDFYMNWIRQPAGKAPEWLGLIRNKTKGYTTEYNTSVKGRFTISRDNTQNMLYLQMNSLRPEDTATYYCARIGVNNGGSLDYWGQGVMVTVSS

SEQ ID NO: 107 - protein, 113 aa, Artificial Sequence; identical to SEQ ID NO: 83
  Synthetic Construct

SEQ ID NO: 108 - protein, 8 aa, Artificial Sequence
  Synthetic Construct
GFTFTDFY

SEQ ID NO: 109 - protein, 10 aa, Artificial Sequence
  Synthetic Construct
IRNKANGYTT

SEQ ID NO: 110 - protein, 15 aa, Artificial Sequence
  Synthetic Construct
ARIGINNGGSLDYWG

SEQ ID NO: 111 - protein, 12 aa, Artificial Sequence
  Synthetic Construct
QSLLYSEKNQDY

SEQ ID NO: 112 - protein, 3 aa, Artificial Sequence
  Synthetic Construct
GAS

SEQ ID NO: 113 - protein, 9 aa, Artificial Sequence
  Synthetic Construct
EQTYSYPYT

SEQ ID NO: 114 - protein, 122 aa, Artificial Sequence; identical to SEQ ID NO: 98
  Synthetic Construct

SEQ ID NO: 115 - protein, 113 aa, Artificial Sequence; identical to SEQ ID NO: 99
  Synthetic Construct

SEQ ID NO: 116 - protein, 8 aa, Artificial Sequence
  Synthetic Construct
GFTFTDFY

SEQ ID NO: 117 - protein, 10 aa, Artificial Sequence
  Synthetic Construct
IRNKTKGYTT

SEQ ID NO: 118 - protein, 15 aa, Artificial Sequence
  Synthetic Construct
ARIGTNNGGSLDYWG

SEQ ID NO: 119 - protein, 12 aa, Artificial Sequence
  Synthetic Construct
QSLLYSENNQDY

SEQ ID NO: 120 - protein, 3 aa, Artificial Sequence
  Synthetic Construct
GAS

SEQ ID NO: 121 - protein, 9 aa, Artificial Sequence
  Synthetic Construct
EQTYSYPYT

SEQ ID NO: 122 - protein, 122 aa, Artificial Sequence
  Synthetic Construct
EVKLLESGGGLVQPGGSMRLSCAASGFTFTDFYMNWIRQPAGETPEWLGLIRNKTKGYTTEYNPSVKGRFTISRDNTQNMLYLQMNSLRPEDTATYYCARIGTNNGGSLDYWGQGVMVTVSS

SEQ ID NO: 123 - protein, 113 aa, Artificial Sequence; identical to SEQ ID NO: 83
  Synthetic Construct

SEQ ID NO: 124 - protein, 8 aa, Artificial Sequence
  Synthetic Construct
GFTFTDFY

SEQ ID NO: 125 - protein, 10 aa, Artificial Sequence
  Synthetic Construct
IRNKVNGYRT

SEQ ID NO: 126 - protein, 15 aa, Artificial Sequence
  Synthetic Construct
ARIGINNGGSLDYWG

SEQ ID NO: 127 - protein, 12 aa, Artificial Sequence
  Synthetic Construct
QSLLYSENNQDY

SEQ ID NO: 128 - protein, 3 aa, Artificial Sequence
  Synthetic Construct
GAS

SEQ ID NO: 129 - protein, 9 aa, Artificial Sequence
  Synthetic Construct
EQTYSYPYT

SEQ ID NO: 130 - protein, 122 aa, Artificial Sequence
  Synthetic Construct
EVKLLESGGGLVQPGGSMRLSCVVSGFTFTDFYMNWIRQAAGKAPEWLGLIRNKVNGYRTEYNPSVKGRFTISRDNIQNMLYLQMNTLRAEDTATYYCARIGINNGGSLDYWGQGVMVTVSS

SEQ ID NO: 131 - protein, 113 aa, Artificial Sequence; identical to SEQ ID NO: 75
  Synthetic Construct

SEQ ID NO: 132 - protein, 8 aa, Artificial Sequence
  Synthetic Construct
GFTFTDFY

SEQ ID NO: 133 - protein, 10 aa, Artificial Sequence
  Synthetic Construct
IRNKAYGYTT

SEQ ID NO: 134 - protein, 15 aa, Artificial Sequence
  Synthetic Construct
ARIGINYGGSLDYWG

SEQ ID NO: 135 - protein, 12 aa, Artificial Sequence
  Synthetic Construct
QSLLYSESNQDY

SEQ ID NO: 136 - protein, 3 aa, Artificial Sequence
  Synthetic Construct
GAS

SEQ ID NO: 137 - protein, 9 aa, Artificial Sequence
  Synthetic Construct
EQTYSYPYT

SEQ ID NO: 138 - protein, 122 aa, Artificial Sequence
  Synthetic Construct
EVKLLESGGGLVQPGGSMRLSCAASGFTFTDFYMNWIRQPAGKAPEWLGLIRNKAYGYTTEYNPSVKGRFTISRDNTQDMLYLQMNTLRAEDTATYYCARIGINYGGSLDYWGQGVMVTVSS

SEQ ID NO: 139 - protein, 113 aa, Artificial Sequence
  Synthetic Construct
DILINQSPASLTVSAGEKVTVSCKSSQSLLYSESNQDYLAWYQQKPGQFPKLLIYGASYRHTGVPDRFTGSGSGTDFTLTISSVQAEDLAHYYCEQTYSYPYTFGAGTKLELK

SEQ ID NO: 140 - protein, 8 aa, Artificial Sequence
  Synthetic Construct
GFTFTDFY

SEQ ID NO: 141 - protein, 10 aa, Artificial Sequence
  Synthetic Construct
IRNKANGFTT

SEQ ID NO: 142 - protein, 15 aa, Artificial Sequence
  Synthetic Construct
ARIGINNGGSLDYWG

SEQ ID NO: 143 - protein, 12 aa, Artificial Sequence
  Synthetic Construct
QSLLYSENKQDY

SEQ ID NO: 144 - protein, 3 aa, Artificial Sequence
  Synthetic Construct
GAS

SEQ ID NO: 145 - protein, 9 aa, Artificial Sequence
  Synthetic Construct
EQTYSYPYT

SEQ ID NO: 146 - protein, 122 aa, Artificial Sequence
  Synthetic Construct
EVKLLESGGGLVQPGGSLRLSCVASGFTFTDFYMNWIRQPAGKAPEWLGLIRNKANGFTTEYNPSVKGRFTISRDNTQHMLYLQMNTLRAEDTATYYCARIGINNGGSLDYWGQGVMVTVSS

SEQ ID NO: 147 - protein, 113 aa, Artificial Sequence
  Synthetic Construct
DILINQSPASLTVSTGEKVTMSCKSSQSLLYSENKQDYLAWYQQKPGQFPKLLIYGASNRHTGVPDRFTGSGSGTDFTLTINIVQAEDLADYYCEQTYSYPYTFGAGTKLELK

SEQ ID NO: 148 - protein, 107 aa, Artificial Sequence
  Synthetic Construct
DIQMTQSPSSVSASVGDRVTITCRASQGISSWLAWYQQKPGKAPKLLIYAASSLQNGVPSRFSGSGSGTDFTLTISSLQPEDFATYFCQQADSFPRTFGQGTKLEIK

SEQ ID NO: 149 - protein, 123 aa, Artificial Sequence
  Synthetic Construct
EVQLVQSGAEVKKPGESLKISCKGSGYSFTSYWIAWVRQMPGKGLEWMGIIYPGDSDTRYSPSFQGQVTISADKSISTAYLQWSSLKASDTAMYFCARQRTFYYDSSDYFDYWGQGTLVTVSS

SEQ ID NO: 150 - protein, 107 aa, Artificial Sequence
  Synthetic Construct
DIQMTQSPSSLSASVGDRVTITCRASQGISNYLAWFQQKPGKAPKSLIYAASSLQSGVPSKFSGSGSGTDFTLTISSLQPEDFATYYCQQYSTYPFTFGPGTKVDIK

SEQ ID NO: 151 - protein, 121 aa, Artificial Sequence
  Synthetic Construct
QVQLVQSGAEVKKPGASVKVSCKASGYTFTGYYIHWVRQAPGLGLEWMGWINPYSGGTTSAQKFQGRVTMTRDTSISSAYMELSRLRSDDTAVYYCARDGGYLALYGTDVWGQGTTVTVSS

SEQ ID NO: 152 - protein, 113 aa, Artificial Sequence
  Synthetic Construct
DIVMTQSPDSLAVSLGERATINCKSSQSVLYSSNNKHFLAWYQQKPGQPPKLLIYWASTRESGVPDRFSGSGSGTDFTLTISSLQAEDVAFYYCQQYYSTPLTFGGGTKVEIK

SEQ ID NO: 153 - protein, 119 aa, Artificial Sequence
  Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAYTPMAFFDYWGQGTLVTVSS

SEQ ID NO: 154 - protein, 107 aa, Artificial Sequence
  Synthetic Construct
EKVMTQSPATLSVSPGERATLSCRASQSVNNNLAWYQQKPGQAPRLLIYGASTRATGIPARFSGSGSGTEFTLTISSLQSEDFAVYYCQQYNNWPRTFGQGTKVEIK

SEQ ID NO: 155 - protein, 124 aa, Artificial Sequence
  Synthetic Construct
QVQLQQWGAGLLKPSETLSLTCAVYGGSFSSYYWSWIRQPPGKGLEWIGEINHSGNTNYNPSLKSRVTISVDTSKNQFSLKLSSVTAADTAVYYCAREGYYDILTGYHDAFDIWDQGTMVTVFS

SEQ ID NO: 156 - protein, 11 aa, Artificial Sequence
  Synthetic Construct
RASQSVSSNLA

SEQ ID NO: 157 - protein, 7 aa, Artificial Sequence
  Synthetic Construct
GASTRAT

SEQ ID NO: 158 - protein, 9 aa, Artificial Sequence
  Synthetic Construct
LQDNNWPPT

SEQ ID NO: 159 - protein, 5 aa, Artificial Sequence
  Synthetic Construct
SYWIG

SEQ ID NO: 160 - protein, 17 aa, Artificial Sequence
  Synthetic Construct
IIYPGDSDTRYSPSFQG

SEQ ID NO: 161 - protein, 12 aa, Artificial Sequence
  Synthetic Construct
RRQGIWGDALDF

SEQ ID NO: 162 - protein, 107 aa, Artificial Sequence
  Synthetic Construct
EIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIK

SEQ ID NO: 163 - protein, 121 aa, Artificial Sequence
  Synthetic Construct
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSS

SEQ ID NO: 164 - protein, 107 aa, Artificial Sequence
  Synthetic Construct
EIVMTQSPATLSVSPGERATLSCRASQSVSSNLAWFQQKPGQAPRLLIYGASTRATGIPARFSVSGSGTEFTLTISSLQSEDFAVYYCLQDNNWPPTFGPGTKVDIK

SEQ ID NO: 165 - protein, 121 aa, Artificial Sequence
  Synthetic Construct
EVQLVQSGAEVKKPGESLMISCKGSGYSFTSYWIGWVRQMPGKGLEWMGIIYPGDSDTRYSPSFQGQVTISADKSISTAYLQWSSLKASDTAMYFCARRRQGIWGDALDFWGQGTLVTVSS

SEQ ID NO: 166 - protein, 11 aa, Artificial Sequence
  Synthetic Construct
RASQGISSWLA

SEQ ID NO: 167 - protein, 7 aa, Artificial Sequence
  Synthetic Construct
AASSLQN

SEQ ID NO: 168 - protein, 9 aa, Artificial Sequence
  Synthetic Construct
QQADSFPRT

SEQ ID NO: 169 - protein, 5 aa, Artificial Sequence
  Synthetic Construct
SYWIA

SEQ ID NO: 170 - protein, 17 aa, Artificial Sequence
  Synthetic Construct
IIYPGDSDTRYSPSFQG

SEQ ID NO: 171 - protein, 14 aa, Artificial Sequence
  Synthetic Construct
QRTFYYDSSDYFDY

SEQ ID NO: 172 - protein, 11 aa, Artificial Sequence
  Synthetic Construct
RASQGISNYLA

SEQ ID NO: 173 - protein, 7 aa, Artificial Sequence
  Synthetic Construct
AASSLQS

SEQ ID NO: 174 - protein, 9 aa, Artificial Sequence
  Synthetic Construct
QQYSTYPFT

SEQ ID NO: 175 - protein, 5 aa, Artificial Sequence
  Synthetic Construct
GYYIH

SEQ ID NO: 176 - protein, 17 aa, Artificial Sequence
  Synthetic Construct
WINPYSGGTTSAQKFQG

SEQ ID NO: 177 - protein, 12 aa, Artificial Sequence
  Synthetic Construct
DGGYLALYGTDV

SEQ ID NO: 178 - protein, 17 aa, Artificial Sequence
  Synthetic Construct
KSSQSVLYSSNNKHFLA

SEQ ID NO: 179 - protein, 7 aa, Artificial Sequence
  Synthetic Construct
WASTRES

SEQ ID NO: 180 - protein, 9 aa, Artificial Sequence
  Synthetic Construct
QQYYSTPLT

SEQ ID NO: 181 - protein, 5 aa, Artificial Sequence
  Synthetic Construct
SYAMS

SEQ ID NO: 182 - protein, 17 aa, Artificial Sequence
  Synthetic Construct
AISGSGGSTYYADSVKG

SEQ ID NO: 183 - protein, 10 aa, Artificial Sequence
  Synthetic Construct
AYTPMAFFDY

SEQ ID NO: 184 - protein, 11 aa, Artificial Sequence
  Synthetic Construct
RASQSVNNNLA

SEQ ID NO: 185 - protein, 7 aa, Artificial Sequence
  Synthetic Construct
GASTRAT

SEQ ID NO: 186 - protein, 9 aa, Artificial Sequence
  Synthetic Construct
QQYNNWPRT

SEQ ID NO: 187 - protein, 5 aa, Artificial Sequence
  Synthetic Construct
SYYWS

SEQ ID NO: 188 - protein, 16 aa, Artificial Sequence
  Synthetic Construct
EINHSGNTNYNPSLKS

SEQ ID NO: 189 - protein, 16 aa, Artificial Sequence
  Synthetic Construct
EGYYDILTGYHDAFDI

(19) United States
(12) Patent Application Publication
(10) Pub. No.: US 2023/0159637 A1
(43) Pub. Date: May 25, 2023

(54) METHODS OF USE OF ANTI-TREM2 ANTIBODIES

(71) Applicants: Alector LLC, South San Francisco, CA (US); Washington University, St. Louis, MO (US)

(72) Inventors: Francesca CIGNARELLA, South San Francisco, CA (US); Fabia FILIPELLO, St. Louis, MO (US); Bryan BOLLMAN, St. Louis, MO (US); Arnon ROSENTHAL, Woodside, CA (US); Tina SCHWABE, San Francisco, CA (US); Ilaria TASSI, San Francisco, CA (US); Laura PICCIO, St. Louis, MO (US)

(73) Assignees: Alector LLC, South San Francisco, CA (US); Washington University, St. Louis, MO (US)

(21) Appl. No.: 17/801,776

(22) PCT Filed: Feb. 23, 2021
(86) PCT No.: PCT/US2021/019260

§ 371 Date: Aug. 23, 2022

Related U.S. Application Data
(60) Provisional application No. 62/980,929, filed on Feb. 24, 2020.

(51) Int. Cl. C07K 16/28; A61P 25/28

(52) U.S. Cl. CPC C07K 16/2803; A61P 25/28; A61K 2039/505

(57) ABSTRACT

The present disclosure is generally directed to the use of anti-TREM2 antibodies in preventing, reducing risk, or treating disease in an individual in need thereof.

### CROSS-REFERENCE TO RELATED APPLICATION

This application claims the benefit of U.S. Provisional Application 62/980,929, filed Feb. 24, 2020, which is hereby incorporated by reference in its entirety.

### FIELD OF THE PRESENT DISCLOSURE

The present disclosure relates to therapeutic uses of anti-TREM2 antibodies.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 735022003440SEQLIST.TXT, date recorded: Feb. 23, 2021, size: 108 KB).

### BACKGROUND OF THE PRESENT DISCLOSURE

Multiple sclerosis (MS) is a chronic demyelinating disease of the central nervous system (CNS) whose pathogenesis involves inflammatory and neurodegenerative processes. The pathological hallmark in MS is the formation of demyelinating lesions in the white and gray matter (Lassmann, Bruck et al. 2007). Oligodendrocyte (OL) loss during demyelination is followed by remyelination primarily sustained by oligodendrocyte precursor cells (OPCs) that differentiate into mature OLs. In MS, impaired generation of OLs leads to an imbalance between demyelination and remyelination. The lack of remyelination in MS eventually leads to axonal damage which clinically manifests as neurological disability (Dulamea (2017) Adv Exp Med Biol, 958:91-127).

Resident microglial cells and infiltrating monocytes/macrophages are thought to play a dual role in demyelination lesions, depending on the phase of lesion formation. On one end, they could contribute to myelin damage and lesion expansion; on the other, they may support generation of OLs and promote remyelination of such lesions by clearing myelin debris, dampening inflammation, and secreting regenerative factors (Kotter, Li et al. 2006; Lampron, Larochelle et al. 2015; Franklin and French-Constant 2017; Voet, Prinz et al. 2019). TREM2 is an important modulator of microglia functions in the CNS (Cantoni, Bollman et al. 2015; Poliani, Wang et al. 2015; Kiialainen et al., 2005; Schmid et al., 2002). Mutations in TREM2 have been associated with neurodegenerative diseases such as Nasu-Hakola disease (NHD), a rare genetic disorder characterized by loss of myelin and axons in the brain (Klunemann, Ridha et al. 2005), Alzheimer's disease, frontotemporal dementia, Parkinson's disease, and amyotrophic lateral sclerosis (Guerreiro, Wojtas et al. 2012, Jonsson, Stefansson et al. 2012, Rayaprolu, Mullen et al. 2013, Cady, Koval et al. 2014).

Current treatments for demyelination diseases such as MS target the inflammatory disease component, thereby reducing attack frequency and severity. However, no therapies are presently available to regenerate myelin and to halt disease progression. Accordingly, there is a need in the art for treatments that promote remyelination in MS and other demyelinating diseases.

All references cited herein, including patent applications and publications, are hereby incorporated by reference in their entirety.

### SUMMARY OF THE PRESENT DISCLOSURE

The present disclosure is generally directed to methods of treating individuals having a demyelination disease comprising administering to the individual an antibody that binds to a TREM2 protein, where the antibody is an agonist.

In one aspect, provided herein is a method for treating or preventing a central nervous system (CNS) demyelination disease, comprising administering to an individual in need thereof a therapeutically effective amount of an agonist antibody that binds to a TREM2 protein. In some embodiments, the antibody promotes remyelination in one or more demyelination lesions in the CNS of the individual.

In another aspect, provided herein is a method for promoting remyelination of one or more demyelination lesions in an individual having a central nervous system (CNS) demyelination disease, comprising administering to the individual a therapeutically effective amount of an agonist antibody that binds to a TREM2 protein.

In some embodiments, which may be combined with any of the preceding embodiments, the TREM2 protein is a mammalian protein or a human protein. In some embodiments, the TREM2 protein is a wild-type protein, a naturally occurring variant, or a disease variant.

In some embodiments, which may be combined with any of the preceding embodiments, the antibody induces one or more TREM2 activities selected from: (a) TREM2 binding to DAP12; (b) DAP12 phosphorylation; (c) activation of Syk kinase; (d) recruitment of Syk to a DAP12/TREM2 complex; or (e) increasing activity of one or more TREM2-dependent genes, optionally wherein the one or more TREM2-dependent genes comprise nuclear factor of activated T-cells (NFAT) transcription factors.

In some embodiments, which may be combined with any of the preceding embodiments, the antibody enhances one or more TREM2 activities that are induced in the presence of myelin. In some embodiments, the one or more TREM2 activities induced in the presence of myelin comprise increasing activity of one or more TREM2-dependent genes, optionally wherein the one or more TREM2-dependent genes comprise nuclear factor of activated T-cells (NFAT) transcription factors.

In some embodiments, which may be combined with any of the preceding embodiments, the antibody promotes recruitment of oligodendrocyte precursor cells (OPCs) to one or more demyelination lesions in the CNS of the individual. In some embodiments, the OPCs are PDGFRα positive.

In some embodiments, which may be combined with any of the preceding embodiments, the antibody promotes an increase of mature oligodendrocytes (OLs) in one or more demyelination lesions in the CNS of the individual. In some embodiments, the antibody promotes differentiation of OPCs into mature oligodendrocytes (OLs) in one or more demyelination lesions in the CNS of the individual. In some embodiments, the mature OLs are OLIG2 and/or CNPase positive.

In some embodiments, which may be combined with any of the preceding embodiments, the antibody promotes an increase in the levels of phosphorylated neurofilaments in one or more demyelination lesions in the CNS of the individual. In some embodiments, the phosphorylated neurofilaments are SMI-31 positive.

In some embodiments, which may be combined with any of the preceding embodiments, the antibody promotes clearance of myelin debris in one or more demyelination lesions in the individual.

In some embodiments, which may be combined with any of the preceding embodiments, the antibody is a murine antibody, a humanized antibody, a bispecific antibody, a multivalent antibody, a conjugated antibody, or a chimeric antibody. In some embodiments, the antibody is a monoclonal antibody. In some embodiments, the antibody binds to one or more amino acids within amino acid residues 124-153 of SEQ ID NO: 1, or amino acid residues on a TREM2 protein corresponding to amino acid residues 124-153 of SEQ ID NO: 1; within amino acid residues 129-153 of SEQ ID NO: 1, or amino acid residues on a TREM2 protein corresponding to amino acid residues 129-153 of SEQ ID NO: 1; within amino acid residues 140-149 of SEQ ID NO: 1, or amino acid residues on a TREM2 protein corresponding to amino acid residues 140-149 of SEQ ID NO: 1; within amino acid residues 149-157 of SEQ ID NO: 1, or amino acid residues on a TREM2 protein corresponding to amino acid residues 149-157 of SEQ ID NO: 1; or within amino acid residues 153-162 of SEQ ID NO: 1, or amino acid residues on a TREM2 protein corresponding to amino acid residues 153-162 of SEQ ID NO: 1. In some embodiments, the antibody binds to one or more amino acid residues selected from D140, L141, W142, F143, P144, E151, D152, H154, E156, or H157 of SEQ ID NO: 1, or one or more amino acid residues on a mammalian TREM2 protein corresponding to an amino acid residue selected from D140, L141, W142, F143, P144, E151, D152, H154, E156, or H157 of SEQ ID NO: 1.

In some embodiments, which may be combined with any of the preceding embodiments, the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSQWMN (SEQ ID NO: 34), the HVR-H2 comprises the amino acid sequence RIYPGGGDTNYAGKFQG (SEQ ID NO: 35), the HVR-H3 comprises the amino acid sequence ARLLRNQPGESYAMDY (SEQ ID NO: 31), the HVR-L1 comprises the amino acid sequence RSSQSLVHSNRYTYLH (SEQ ID NO: 41), the HVR-L2 comprises the amino acid sequence KVSNRFS (SEQ ID NO: 33), and the HVR-L3 comprises the amino acid sequence SQSTRVPYT (SEQ ID NO: 32). In some embodiments, the antibody comprises a heavy chain variable region comprising the amino acid sequence of SEQ ID NO: 27 and a light chain variable region comprising the amino acid sequence of SEQ ID NO: 30. In some embodiments, the antibody comprises: (a) a heavy chain comprising the amino acid sequence of SEQ ID NO: 43, and a light chain comprising the amino acid sequence of SEQ ID NO: 47; or (b) a heavy chain comprising the amino acid sequence of SEQ ID NO: 44, and a light chain comprising the amino acid sequence of SEQ ID NO: 47.

In some embodiments, which may be combined with any of the preceding embodiments, the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSDWMN (SEQ ID NO: 36), the HVR-H2 comprises the amino acid sequence RIYPGEGDTNYARKFHG (SEQ ID NO: 37), the HVR-H3 comprises the amino acid sequence ARLLRNKPGESYAMDY (SEQ ID NO: 38), the HVR-L1 comprises the amino acid sequence RTSQSLVHSNAYTYLH (SEQ ID NO: 39), the HVR-L2 comprises the amino acid sequence KVSNRVS (SEQ ID NO: 40), and the HVR-L3 comprises the amino acid sequence SQSTRVPYT (SEQ ID NO: 32). In some embodiments, the antibody comprises a heavy chain variable region comprising the amino acid sequence of SEQ ID NO: 28 and a light chain variable region comprising the amino acid sequence of SEQ ID NO: 29. In some embodiments, the antibody comprises: (a) a heavy chain comprising the amino acid sequence of SEQ ID NO: 45, and a light chain comprising the amino acid sequence of SEQ ID NO: 48; or (b) a heavy chain comprising the amino acid sequence of SEQ ID NO: 46, and a light chain comprising the amino acid sequence of SEQ ID NO: 48.

In some embodiments, which may be combined with any of the preceding embodiments, the antibody is a fragment and the fragment is a Fab, Fab′, Fab′-SH, F(ab′)2, Fv or scFv fragment. In some embodiments, the antibody is of the IgG class, the IgM class, or the IgA class. In some embodiments, the antibody is of the IgG class and has an IgG1, IgG2, IgG3, or IgG4 isotype. In some embodiments, the antibody has a human IgG1 isotype and comprises amino acid substitutions in the Fc region at the residue positions P331S and E430G, wherein the numbering of the residues is according to EU numbering.

In some embodiments, which may be combined with any of the preceding embodiments, the individual is a human. In some embodiments, the individual comprises at least one copy of a functional TREM2 gene. In some embodiments, the individual is heterozygous for a mutation in a TREM2 gene. In some embodiments, the individual is homozygous for a mutation in a TREM2 gene.

In some embodiments, which may be combined with any of the preceding embodiments, the individual has or is at risk for a disease characteristic selected from myelin damage, one or more demyelination lesions in the CNS, inflammation in the CNS, one or more plaques in the CNS, loss of myelin sheaths, axonal damage, reduced OPCs, reduced OLs, reduced myelin debris clearance, axonal varicosities, axonal spheroids, gliosis, autofluorescent lipid-laden macrophages, axon destruction, or any combination thereof. In some embodiments, the individual has or is at risk for having axonal damage and/or one or more demyelination lesions in the CNS. In some embodiments, the axonal damage and/or the one or more demyelination lesions in the CNS are in the white matter, the gray matter, or the corpus callosum of the CNS.

In some embodiments, which may be combined with any of the preceding embodiments, the individual has or is at risk for having a symptom selected from changes in sensation, pricking, numbness, muscle weakness, clonus, muscle spasms, difficulty in moving, difficulties with coordination, difficulties with balance, problems in speech, problems in swallowing, visual problems, fatigue, acute pain, chronic pain, bladder difficulties, bowel difficulties, cognitive impairment, depression, unstable mood, Uhthoffs phenomenon, Lhermitte's sign, or any combination thereof.

In some embodiments, which may be combined with any of the preceding embodiments, the demyelination disease or disorder is multiple sclerosis, optic neuritis, neuromyelitis optica (Devic's disease), transverse myelitis, acute disseminated encephalomyelitis, adrenoleukodystrophy, or adrenomyeloneuropathy. In some embodiments, the demyelination disease or disorder is multiple sclerosis.

In some embodiments, which may be combined with any of the preceding embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 52; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 53; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 54; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 55; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 56; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 57. In some embodiments, which may be combined with any of the preceding embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 52; (b) an HVR-H2 comprising the amino acid sequence of SEQ ID NO: 53; and (c) an HVR-H3 comprising the amino acid sequence of SEQ ID NO: 54; and wherein the light chain variable domain comprises: (a) an HVR-L1 comprising the amino acid sequence of SEQ ID NO: 55; (b) an HVR-L2 comprising the amino acid sequence of SEQ ID NO: 56; and (c) an HVR-L3 comprising the amino acid sequence of SEQ ID NO: 57.

In some embodiments, which may be combined with any of the preceding embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 60; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 61; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 62; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 63; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 64; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 65. In some embodiments, which may be combined with any of the preceding embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 60; (b) an HVR-H2 comprising the amino acid sequence of SEQ ID NO: 61; and (c) an HVR-H3 comprising the amino acid sequence of SEQ ID NO: 62; and wherein the light chain variable domain comprises: (a) an HVR-L1 comprising the amino acid sequence of SEQ ID NO: 63; (b) an HVR-L2 comprising the amino acid sequence of SEQ ID NO: 64; and (c) an HVR-L3 comprising the amino acid sequence of SEQ ID NO: 65.

In some embodiments, which may be combined with any of the preceding embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 68; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 69; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 70; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 71; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 72; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 73. In some embodiments, which may be combined with any of the preceding embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 68; (b) an HVR-H2 comprising the amino acid sequence of SEQ ID NO: 69; and (c) an HVR-H3 comprising the amino acid sequence of SEQ ID NO: 70; and wherein the light chain variable domain comprises: (a) an HVR-L1 comprising the amino acid sequence of SEQ ID NO: 71; (b) an HVR-L2 comprising the amino acid sequence of SEQ ID NO: 72; and (c) an HVR-L3 comprising the amino acid sequence of SEQ ID NO: 73.

In some embodiments, which may be combined with any of the preceding embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 159; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 160; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 161; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 156; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 157; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 158. In some embodiments, which may be combined with any of the preceding embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 159; (b) an HVR-H2 comprising the amino acid sequence of SEQ ID NO: 160; and (c) an HVR-H3 comprising the amino acid sequence of SEQ ID NO: 161; and wherein the light chain variable domain comprises: (a) an HVR-L1 comprising the amino acid sequence of SEQ ID NO: 156; (b) an HVR-L2 comprising the amino acid sequence of SEQ ID NO: 157; and (c) an HVR-L3 comprising the amino acid sequence of SEQ ID NO: 158. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 169; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 170; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 171; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 166; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 167; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 168. In some embodiments, which may be combined with any of the preceding embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 169; (b) an HVR-H2 comprising the amino acid sequence of SEQ ID NO: 170; and (c) an HVR-H3 comprising the amino acid sequence of SEQ ID NO: 171; and wherein the light chain variable domain comprises: (a) an HVR-L1 comprising the amino acid sequence of SEQ ID NO: 166; (b) an HVR-L2 comprising the amino acid sequence of SEQ ID NO: 167; and (c) an HVR-L3 comprising the amino acid sequence of SEQ ID NO: 168. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 175; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 176; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 177; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 172; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 173; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 174. In some embodiments, which may be combined with any of the preceding embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 175; (b) an HVR-H2 comprising the amino acid sequence of SEQ ID NO: 176; and (c) an HVR-H3 comprising the amino acid sequence of SEQ ID NO: 177; and wherein the light chain variable domain comprises: (a) an HVR-L1 comprising the amino acid sequence of SEQ ID NO: 172; (b) an HVR-L2 comprising the amino acid sequence of SEQ ID NO: 173; and (c) an HVR-L3 comprising the amino acid sequence of SEQ ID NO: 174. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 181; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 182; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 183; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 178; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 179; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 180. In some embodiments, which may be combined with any of the preceding embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 181; (b) an HVR-H2 comprising the amino acid sequence of SEQ ID NO: 182; and (c) an HVR-H3 comprising the amino acid sequence of SEQ ID NO: 183; and wherein the light chain variable domain comprises: (a) an HVR-L1 comprising the amino acid sequence of SEQ ID NO: 178; (b) an HVR-L2 comprising the amino acid sequence of SEQ ID NO: 179; and (c) an HVR-L3 comprising the amino acid sequence of SEQ ID NO: 180. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 187; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 188; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 189; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 184; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 185; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 186. In some embodiments, which may be combined with any of the preceding embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 187; (b) an HVR-H2 comprising the amino acid sequence of SEQ ID NO: 188; and (c) an HVR-H3 comprising the amino acid sequence of SEQ ID NO: 189; and wherein the light chain variable domain comprises: (a) an HVR-L1 comprising the amino acid sequence of SEQ ID NO: 184; (b) an HVR-L2 comprising the amino acid sequence of SEQ ID NO: 185; and (c) an HVR-L3 comprising the amino acid sequence of SEQ ID NO: 186. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 58; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 59. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 58, wherein the heavy chain variable domain comprises the HVR-H1, HVR-H2, and HVR-H3 amino acid sequences of antibody 42E8.H1. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 59, wherein the light chain variable domain comprises the HVR-L1, HVR-L2, and HVR-L3 amino acid sequences of antibody 42E8.H1. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 58 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 42E8.H1 or the amino acid sequence of SEQ ID NO: 58. In certain embodiments, a total of 1 to 5 amino acids have been substituted, inserted and/or deleted in the heavy chain variable domain amino acid sequence of antibody 42E8.H1 or the amino acid sequence of SEQ ID NO: 58. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody 42E8.H1 or of SEQ ID NO: 58, including post-translational modifications of that sequence. In a particular embodiment, the VH comprises one, two or three HVRs selected from: (a) the HVR-H1 amino acid sequence of antibody 42E8.H1, (b) the HVR-H2 amino acid sequence of antibody 42E8.H1, and (c) the HVR-H3 amino acid sequence of antibody 42E8.H1. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 59 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 42E8.H1 or the amino acid sequence of SEQ ID NO: 59. In certain embodiments, a total of 1 to 5 amino acids have been substituted, inserted and/or deleted in the light chain variable domain amino acid sequence of antibody 42E8.H1 or the amino acid sequence of SEQ ID NO: 59. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody 42E8.H1 or of SEQ ID NO: 59, including post-translational modifications of that sequence. In a particular embodiment, the VL comprises one, two or three HVRs selected from: (a) the HVR-L1 amino acid sequence of antibody 42E8.H1, (b) the HVR-L2 amino acid sequence of antibody 42E8.H1, and (c) the HVR-L3 amino acid sequence of antibody 42E8.H1. In some embodiments, the antibody comprises a heavy chain variable region comprising the amino acid sequence of SEQ ID NO: 58 and a light chain variable region comprising the amino acid sequence of SEQ ID NO: 59.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 66; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 67. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 66, wherein the heavy chain variable domain comprises the HVR-H1, HVR-H2, and HVR-H3 amino acid sequences of antibody RS9.F6. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 67, wherein the light chain variable domain comprises the HVR-L1, HVR-L2, and HVR-L3 amino acid sequences of antibody RS9.F6. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 66 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody RS9.F6 or the amino acid sequence of SEQ ID NO: 66. In certain embodiments, a total of 1 to 5 amino acids have been substituted, inserted and/or deleted in the heavy chain variable domain amino acid sequence of antibody RS9.F6 or the amino acid sequence of SEQ ID NO: 66. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody RS9.F6 or of SEQ ID NO: 66, including post-translational modifications of that sequence. In a particular embodiment, the VH comprises one, two or three HVRs selected from: (a) the HVR-H1 amino acid sequence of antibody RS9.F6, (b) the HVR-H2 amino acid sequence of antibody RS9.F6, and (c) the HVR-H3 amino acid sequence of antibody RS9.F6. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 67 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody RS9.F6 or the amino acid sequence of SEQ ID NO: 67. In certain embodiments, a total of 1 to 5 amino acids have been substituted, inserted and/or deleted in the light chain variable domain amino acid sequence of antibody RS9.F6 or the amino acid sequence of SEQ ID NO: 67. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody RS9.F6 or of SEQ ID NO: 67, including post-translational modifications of that sequence. In a particular embodiment, the VL comprises one, two or three HVRs selected from: (a) the HVR-L1 amino acid sequence of antibody RS9.F6, (b) the HVR-L2 amino acid sequence of antibody RS9.F6, and (c) the HVR-L3 amino acid sequence of antibody RS9.F6. In some embodiments, the antibody comprises a heavy chain variable region comprising the amino acid sequence of SEQ ID NO: 66 and a light chain variable region comprising the amino acid sequence of SEQ ID NO: 67.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 74; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 75. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 74 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the amino acid sequence of SEQ ID NO: 74. In certain embodiments, a total of 1 to 5 amino acids have been substituted, inserted and/or deleted in the amino acid sequence of SEQ ID NO: 74. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of SEQ ID NO: 74, including post-translational modifications of that sequence. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 75 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the amino acid sequence of SEQ ID NO: 75. In certain embodiments, a total of 1 to 5 amino acids have been substituted, inserted and/or deleted in the amino acid sequence of SEQ ID NO: 75. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of SEQ ID NO: 75, including post-translational modifications of that sequence. In some embodiments, the antibody comprises a heavy chain variable region comprising the amino acid sequence of SEQ ID NO: 74 and a light chain variable region comprising the amino acid sequence of SEQ ID NO: 75.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 6E7 or to the amino acid sequence of SEQ ID NO: 149; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 6E7 or to the amino acid sequence of SEQ ID NO: 148. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 6E7 or to the amino acid sequence of SEQ ID NO: 149, wherein the heavy chain variable domain comprises the HVR-H1, HVR-H2, and HVR-H3 amino acid sequences of antibody 6E7. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 6E7 or to the amino acid sequence of SEQ ID NO: 148, wherein the light chain variable domain comprises the HVR-L1, HVR-L2, and HVR-L3 amino acid sequences of antibody 6E7. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 6E7 or to the amino acid sequence of SEQ ID NO: 149 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 6E7 or the amino acid sequence of SEQ ID NO: 149. In certain embodiments, a total of 1 to 5 amino acids have been substituted, inserted and/or deleted in the heavy chain variable domain amino acid sequence of antibody 6E7 or the amino acid sequence of SEQ ID NO: 149. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 6E7 or of SEQ ID NO: 149, including post-translational modifications of that sequence. In a particular embodiment, the VH comprises one, two or three HVRs selected from: (a) the HVR-H1 amino acid sequence of antibody 6E7, (b) the HVR-H2 amino acid sequence of antibody 6E7, and (c) the HVR-H3 amino acid sequence of antibody 6E7. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 6E7 or to the amino acid sequence of SEQ ID NO: 148 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 6E7 or the amino acid sequence of SEQ ID NO: 148. In certain embodiments, a total of 1 to 5 amino acids have been substituted, inserted and/or deleted in the light chain variable domain amino acid sequence of antibody 6E7 or the amino acid sequence of SEQ ID NO: 148. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 6E7 or of SEQ ID NO: 148, including post-translational modifications of that sequence. In a particular embodiment, the VL comprises one, two or three HVRs selected from: (a) the HVR-L1 amino acid sequence of antibody 6E7, (b) the HVR-L2 amino acid sequence of antibody 6E7, and (c) the HVR-L3 amino acid sequence of antibody 6E7. In some embodiments, the antibody comprises a heavy chain variable region comprising the amino acid sequence of SEQ ID NO: 149 and a light chain variable region comprising the amino acid sequence of SEQ ID NO: 148. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 5E3 or to the amino acid sequence of SEQ ID NO: 151; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 5E3 or to the amino acid sequence of SEQ ID NO: 150. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 5E3 or to the amino acid sequence of SEQ ID NO: 151, wherein the heavy chain variable domain comprises the HVR-H1, HVR-H2, and HVR-H3 amino acid sequences of antibody 5E3. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 5E3 or to the amino acid sequence of SEQ ID NO: 150, wherein the light chain variable domain comprises the HVR-L1, HVR-L2, and HVR-L3 amino acid sequences of antibody 5E3. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 5E3 or to the amino acid sequence of SEQ ID NO: 151 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 5E3 or the amino acid sequence of SEQ ID NO: 151. In certain embodiments, a total of 1 to 5 amino acids have been substituted, inserted and/or deleted in the heavy chain variable domain amino acid sequence of antibody 5E3 or the amino acid sequence of SEQ ID NO: 151. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 5E3 or of SEQ ID NO: 151, including post-translational modifications of that sequence. In a particular embodiment, the VH comprises one, two or three HVRs selected from: (a) the HVR-H1 amino acid sequence of antibody 5E3, (b) the HVR-H2 amino acid sequence of antibody 5E3, and (c) the HVR-H3 amino acid sequence of antibody 5E3. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 5E3 or to the amino acid sequence of SEQ ID NO: 150 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 5E3 or the amino acid sequence of SEQ ID NO: 150. In certain embodiments, a total of 1 to 5 amino acids have been substituted, inserted and/or deleted in the light chain variable domain amino acid sequence of antibody 5E3 or the amino acid sequence of SEQ ID NO: 150. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 5E3 or of SEQ ID NO: 150, including post-translational modifications of that sequence. In a particular embodiment, the VL comprises one, two or three HVRs selected from: (a) the HVR-L1 amino acid sequence of antibody 5E3, (b) the HVR-L2 amino acid sequence of antibody 5E3, and (c) the HVR-L3 amino acid sequence of antibody 5E3. In some embodiments, the antibody comprises a heavy chain variable region comprising the amino acid sequence of SEQ ID NO: 151 and a light chain variable region comprising the amino acid sequence of SEQ ID NO: 150. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 24G6 or to the amino acid sequence of SEQ ID NO: 153; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 24G6 or to the amino acid sequence of SEQ ID NO: 152. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 24G6 or to the amino acid sequence of SEQ ID NO: 153, wherein the heavy chain variable domain comprises the HVR-H1, HVR-H2, and HVR-H3 amino acid sequences of antibody 24G6. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 24G6 or to the amino acid sequence of SEQ ID NO: 152, wherein the light chain variable domain comprises the HVR-L1, HVR-L2, and HVR-L3 amino acid sequences of antibody 24G6. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 24G6 or to the amino acid sequence of SEQ ID NO: 153 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 24G6 or the amino acid sequence of SEQ ID NO: 153. In certain embodiments, a total of 1 to 5 amino acids have been substituted, inserted and/or deleted in the heavy chain variable domain amino acid sequence of antibody 24G6 or the amino acid sequence of SEQ ID NO: 153. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 24G6 or of SEQ ID NO: 153, including post-translational modifications of that sequence. In a particular embodiment, the VH comprises one, two or three HVRs selected from: (a) the HVR-H1 amino acid sequence of antibody 24G6, (b) the HVR-H2 amino acid sequence of antibody 24G6, and (c) the HVR-H3 amino acid sequence of antibody 24G6. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 24G6 or to the amino acid sequence of SEQ ID NO: 152 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 24G6 or the amino acid sequence of SEQ ID NO: 152. In certain embodiments, a total of 1 to 5 amino acids have been substituted, inserted and/or deleted in the light chain variable domain amino acid sequence of antibody 24G6 or the amino acid sequence of SEQ ID NO: 152. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 24G6 or of SEQ ID NO: 152, including post-translational modifications of that sequence. In a particular embodiment, the VL comprises one, two or three HVRs selected from: (a) the HVR-L1 amino acid sequence of antibody 24G6, (b) the HVR-L2 amino acid sequence of antibody 24G6, and (c) the HVR-L3 amino acid sequence of antibody 24G6. In some embodiments, the antibody comprises a heavy chain variable region comprising the amino acid sequence of SEQ ID NO: 153 and a light chain variable region comprising the amino acid sequence of SEQ ID NO: 152. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 25F12 or to the amino acid sequence of SEQ ID NO: 155; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 25F12 or to the amino acid sequence of SEQ ID NO: 154. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 25F12 or to the amino acid sequence of SEQ ID NO: 155, wherein the heavy chain variable domain comprises the HVR-H1, HVR-H2, and HVR-H3 amino acid sequences of antibody 25F12. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 25F12 or to the amino acid sequence of SEQ ID NO: 154, wherein the light chain variable domain comprises the HVR-L1, HVR-L2, and HVR-L3 amino acid sequences of antibody 25F12. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 25F12 or to the amino acid sequence of SEQ ID NO: 155 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 25F12 or the amino acid sequence of SEQ ID NO: 155. In certain embodiments, a total of 1 to 5 amino acids have been substituted, inserted and/or deleted in the heavy chain variable domain amino acid sequence of antibody 25F12 or the amino acid sequence of SEQ ID NO: 155. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 25F12 or of SEQ ID NO: 155, including post-translational modifications of that sequence. In a particular embodiment, the VH comprises one, two or three HVRs selected from: (a) the HVR-H1 amino acid sequence of antibody 25F12, (b) the HVR-H2 amino acid sequence of antibody 25F12, and (c) the HVR-H3 amino acid sequence of antibody 25F12. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 25F12 or to the amino acid sequence of SEQ ID NO: 154 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 25F12 or the amino acid sequence of SEQ ID NO: 154. In certain embodiments, a total of 1 to 5 amino acids have been substituted, inserted and/or deleted in the light chain variable domain amino acid sequence of antibody 25F12 or the amino acid sequence of SEQ ID NO: 154. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 25F12 or of SEQ ID NO: 154, including post-translational modifications of that sequence. In a particular embodiment, the VL comprises one, two or three HVRs selected from: (a) the HVR-L1 amino acid sequence of antibody 25F12, (b) the HVR-L2 amino acid sequence of antibody 25F12, and (c) the HVR-L3 amino acid sequence of antibody 25F12. In some embodiments, the antibody comprises a heavy chain variable region comprising the amino acid sequence of SEQ ID NO: 155 and a light chain variable region comprising the amino acid sequence of SEQ ID NO: 154. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 14C12 or to the amino acid sequence of SEQ ID NO: 165; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 14C12 or to the amino acid sequence of SEQ ID NO: 164. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 14C12 or to the amino acid sequence of SEQ ID NO: 165, wherein the heavy chain variable domain comprises the HVR-H1, HVR-H2, and HVR-H3 amino acid sequences of antibody 13E7 14C12. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 14C12 or to the amino acid sequence of SEQ ID NO: 164, wherein the light chain variable domain comprises the HVR-L1, HVR-L2, and HVR-L3 amino acid sequences of antibody 13E7 14C12. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 14C12 or to the amino acid sequence of SEQ ID NO: 165 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 13E7 14C12 or the amino acid sequence of SEQ ID NO: 165. In certain embodiments, a total of 1 to 5 amino acids have been substituted, inserted and/or deleted in the heavy chain variable domain amino acid sequence of antibody 13E7 14C12 or the amino acid sequence of SEQ ID NO: 165. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 13E7 14C12 or of SEQ ID NO: 165, including post-translational modifications of that sequence. In a particular embodiment, the VH comprises one, two or three HVRs selected from: (a) the HVR-H1 amino acid sequence of antibody 13E7 14C12, (b) the HVR-H2 amino acid sequence of antibody 13E7 14C12, and (c) the HVR-H3 amino acid sequence of antibody 13E7 14C12. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 14C12 or to the amino acid sequence of SEQ ID NO: 164 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 13E7 14C12 or the amino acid sequence of SEQ ID NO: 164. In certain embodiments, a total of 1 to 5 amino acids have been substituted, inserted and/or deleted in the light chain variable domain amino acid sequence of antibody 13E7 14C12 or the amino acid sequence of SEQ ID NO: 164. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 13E7 14C12 or of SEQ ID NO: 164, including post-translational modifications of that sequence. In a particular embodiment, the VL comprises one, two or three HVRs selected from: (a) the HVR-L1 amino acid sequence of antibody 13E7 14C12, (b) the HVR-L2 amino acid sequence of antibody 13E7 14C12, and (c) the HVR-L3 amino acid sequence of antibody 13E7 14C12. In some embodiments, the antibody comprises a heavy chain variable region comprising the amino acid sequence of SEQ ID NO: 165 and a light chain variable region comprising the amino acid sequence of SEQ ID NO: 164. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 (SST202443) or to the amino acid sequence of SEQ ID NO: 163; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 (SST202443) or to the amino acid sequence of SEQ ID NO: 162. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 (SST202443) or to the amino acid sequence of SEQ ID NO: 163, wherein the heavy chain variable domain comprises the HVR-H1, HVR-H2, and HVR-H3 amino acid sequences of antibody 13E7 (SST202443). In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 (SST202443) or to the amino acid sequence of SEQ ID NO: 162, wherein the light chain variable domain comprises the HVR-L1, HVR-L2, and HVR-L3 amino acid sequences of antibody 13E7 (SST202443). In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 (SST202443) or to the amino acid sequence of SEQ ID NO: 163 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 13E7 (SST202443) or the amino acid sequence of SEQ ID NO: 163. In certain embodiments, a total of 1 to 5 amino acids have been substituted, inserted and/or deleted in the heavy chain variable domain amino acid sequence of antibody 13E7 (SST202443) or the amino acid sequence of SEQ ID NO: 163. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 13E7 (SST202443) or of SEQ ID NO: 163, including post-translational modifications of that sequence. In a particular embodiment, the VH comprises one, two or three HVRs selected from: (a) the HVR-H1 amino acid sequence of antibody 13E7 (SST202443), (b) the HVR-H2 amino acid sequence of antibody 13E7 (SST202443), and (c) the HVR-H3 amino acid sequence of antibody 13E7 (SST202443). In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 (SST202443) or to the amino acid sequence of SEQ ID NO: 162 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 13E7 (SST202443) or the amino acid sequence of SEQ ID NO: 162. In certain embodiments, a total of 1 to 5 amino acids have been substituted, inserted and/or deleted in the light chain variable domain amino acid sequence of antibody 13E7 (SST202443) or the amino acid sequence of SEQ ID NO: 162. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 13E7 (SST202443) or of SEQ ID NO: 162, including post-translational modifications of that sequence. In a particular embodiment, the VL comprises one, two or three HVRs selected from: (a) the HVR-L1 amino acid sequence of antibody 13E7 (SST202443), (b) the HVR-L2 amino acid sequence of antibody 13E7 (SST202443), and (c) the HVR-L3 amino acid sequence of antibody 13E7 (SST202443). In some embodiments, the antibody comprises a heavy chain variable region comprising the amino acid sequence of SEQ ID NO: 163 and a light chain variable region comprising the amino acid sequence of SEQ ID NO: 162. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, which may be combined with any of the preceding embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 34; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 35; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 31; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 41; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 33; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 32.

In some embodiments, which may be combined with any of the preceding embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 36; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 37; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 38; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 39; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 40; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 32.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-47 or to the amino acid sequence of SEQ ID NO: 28; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-47 or to the amino acid sequence of SEQ ID NO: 29. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-47 or to the amino acid sequence of SEQ ID NO: 28, wherein the heavy chain variable domain comprises the HVR-H1, HVR-H2, and HVR-H3 amino acid sequences of antibody AL2p-47. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-47 or to the amino acid sequence of SEQ ID NO: 29, wherein the light chain variable domain comprises the HVR-L1, HVR-L2, and HVR-L3 amino acid sequences of antibody AL2p-47. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-47 or to the amino acid sequence of SEQ ID NO: 28 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody AL2p-47 or the amino acid sequence of SEQ ID NO: 28. In certain embodiments, a total of 1 to 5 amino acids have been substituted, inserted and/or deleted in the heavy chain variable domain amino acid sequence of antibody AL2p-47 or the amino acid sequence of SEQ ID NO: 28. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody AL2p-47 or of SEQ ID NO: 28, including post-translational modifications of that sequence. In a particular embodiment, the VH comprises one, two or three HVRs selected from: (a) the HVR-H1 amino acid sequence of antibody AL2p-47, (b) the HVR-H2 amino acid sequence of antibody AL2p-47, and (c) the HVR-H3 amino acid sequence of antibody AL2p-47. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-47 or to the amino acid sequence of SEQ ID NO: 29 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody AL2p-47 or the amino acid sequence of SEQ ID NO: 29. In certain embodiments, a total of 1 to 5 amino acids have been substituted, inserted and/or deleted in the light chain variable domain amino acid sequence of antibody AL2p-47 or the amino acid sequence of SEQ ID NO: 29. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody AL2p-47 or of SEQ ID NO: 29, including post-translational modifications of that sequence. In a particular embodiment, the VL comprises one, two or three HVRs selected from: (a) the HVR-L1 amino acid sequence of antibody AL2p-47, (b) the HVR-L2 amino acid sequence of antibody AL2p-47, and (c) the HVR-L3 amino acid sequence of antibody AL2p-47. In some embodiments, the antibody comprises a heavy chain variable region comprising the amino acid sequence of SEQ ID NO: 28 and a light chain variable region comprising the amino acid sequence of SEQ ID NO: 29.

In some embodiments, which may be combined with any of the preceding embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-58 or to the amino acid sequence of SEQ ID NO: 27; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-58 or to the amino acid sequence of SEQ ID NO: 30. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-58 or to the amino acid sequence of SEQ ID NO: 27, wherein the heavy chain variable domain comprises the HVR-H1, HVR-H2, and HVR-H3 amino acid sequences of antibody AL2p-58. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-58 or to the amino acid sequence of SEQ ID NO: 30, wherein the light chain variable domain comprises the HVR-L1, HVR-L2, and HVR-L3 amino acid sequences of antibody AL2p-58. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-58 or to the amino acid sequence of SEQ ID NO: 27 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody AL2p-58 or the amino acid sequence of SEQ ID NO: 27. In certain embodiments, a total of 1 to 5 amino acids have been substituted, inserted and/or deleted in the heavy chain variable domain amino acid sequence of antibody AL2p-58 or the amino acid sequence of SEQ ID NO: 27. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody AL2p-58 or of SEQ ID NO: 27, including post-translational modifications of that sequence. In a particular embodiment, the VH comprises one, two or three HVRs selected from: (a) the HVR-H1 amino acid sequence of antibody AL2p-58, (b) the HVR-H2 amino acid sequence of antibody AL2p-58, and (c) the HVR-H3 amino acid sequence of antibody AL2p-58. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-58 or to the amino acid sequence of SEQ ID NO: 30 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody AL2p-58 or the amino acid sequence of SEQ ID NO: 30. In certain embodiments, a total of 1 to 5 amino acids have been substituted, inserted and/or deleted in the light chain variable domain amino acid sequence of antibody AL2p-58 or the amino acid sequence of SEQ ID NO: 30. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody AL2p-58 or of SEQ ID NO: 30, including post-translational modifications of that sequence. In a particular embodiment, the VL comprises one, two or three HVRs selected from: (a) the HVR-L1 amino acid sequence of antibody AL2p-58, (b) the HVR-L2 amino acid sequence of antibody AL2p-58, and (c) the HVR-L3 amino acid sequence of antibody AL2p-58. In some embodiments, the antibody comprises a heavy chain variable region comprising the amino acid sequence of SEQ ID NO: 27 and a light chain variable region comprising the amino acid sequence of SEQ ID NO: 30.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIGS. 1A-1C show the results of experiments analyzing myelin debris clearance in the brains of Trem2^{+/+}, Trem2^{-/-} and Trem2^{+/-} mice with cuprizone (CPZ)-induced central nervous system (CNS) demyelination. FIG. 1A provides representative immunohistochemistry images of the corpus callosum of Trem2^{+/+}, Trem2^{-/-} and Trem2^{+/-} mice with CPZ-induced CNS demyelination. An anti-degraded myelin basic protein (dMBP) antibody was used to determine the amount of myelin debris accumulation; an anti-Iba1 antibody was used to image Iba1⁺ microglia; 4′,6-diamidino-2-phenylindole (DAPI) was used to image DNA. FIGS. 1B-1C provide quantifications of the experiment shown in FIG. 1A. In FIG. 1B, the bars show the percent of dMBP+ staining, which was measured as the number of dMBP-positive pixels per mm². In FIG. 1C, the bars show the density of Iba1+ cells, which was measured as the number of Iba1+ cells per mm². In FIGS. 1B-1C, the data shown are the mean±standard deviation (s.d.), with P<0.05 considered statistically significant. The asterisks indicate statistical significance (“*”=p<0.05; “**”=p<0.01; “**”=p<0.001; “**”=p<0.0001).

FIG. 2 shows the results of immunoprecipitation experiments evaluating the effect of anti-TREM2 antibody 7E5 on in vitro TREM2 signaling. FIG. 2 is an immunoblot of DAP12 phosphorylation in TREM2 immunoprecipitates from Trem2^{+/+} and Trem2^{-/-} bone marrow-derived macrophages (BMDMs) that were stimulated in vitro with cross-linked or soluble anti-TREM2 antibody 7E5 or isotype control antibody. DAP12 phosphorylation was analyzed using an anti-phosphotyrosine antibody. Actin immunoblots were used as loading/input controls. In FIG. 2, “x-link” indicates that anti-TREM2 antibody 7E5 or isotype control antibody was cross-linked with a secondary antibody. “Soluble” indicates that crosslinking with a secondary antibody was not performed.

FIGS. 3A-3B show the results of an in vitro TREM2 activity assay using BWZ cells that express mouse TREM2 and DAP12 and were transduced with a luciferase reporter gene under the NFAT promoter control. In FIG. 3A, the BWZ cells were treated with the indicated concentrations of anti-TREM2 antibody 7E5 or isotype control antibody. In FIG. 3B, the BWZ cells were seeded onto the indicated concentrations of myelin and were subsequently treated with anti-TREM2 antibody 7E5 or isotype control antibody. In FIGS. 3A-3B, the y-axis shows the fold luminescence induced by anti-TREM2 antibody 7E5 over the isotype control antibody (fold over control). The data shown are the mean±standard deviation (s.d.), with P<0.05 considered statistically significant. The asterisks indicate statistical significance (“*”=p<0.05; “**”=p<0.01; “***”=p<0.001; “**”=p<0.0001).

FIGS. 4A-4B show the results of immunoprecipitation experiments analyzing the effect of anti-TREM2 antibody 7E5 on in vivo TREM2 signaling. FIG. 4A is an immunoblot of DAP12 phosphorylation in immunoprecipitates of TREM2 from thioglycollate-induced macrophages obtained from C57BL/6 mice treated with anti-TREM2 antibody 7E5 or isotype control antibody. DAP12 phosphorylation was analyzed using an anti-phosphotyrosine antibody. Actin immunoblots were used as loading/input controls. FIG. 4B provides a quantification of the experiment shown in FIG. 4A. The bars indicate the normalized phosphotyrosine signal (i.e., phosphorylated DAP12). The data shown are the mean±standard deviation (s.d.), with P<0.05 considered statistically significant. The asterisks indicate statistical significance (“*”=p<0.05; “**”=p<0.01; “***”=p<0.001; “**”=p<0.0001).

FIGS. 5A-5C show the results of in vivo experiments evaluating the effects of anti-TREM2 antibody 7E5 on myelin debris clearance in the brains of Trem2^{+/-} mice with CPZ-induced CNS demyelination. FIG. 5A is a diagram of the dosing regimen for anti-TREM2 antibody 7E5 or isotype control antibody in Trem2^{+/-} mice. WKS=weeks; ON=active CPZ feeding; OFF=after CPZ feeding; D=days; +3D=3 days after CPZ withdrawal; +7D=7 days after CPZ withdrawal; +14D=14 days after CPZ withdrawal. The asterisks indicate the days on which brain samples were collected for analysis. “INJ”=injection of anti-TREM2 antibody 7E5 or isotype control antibody. FIG. 5B shows representative immunohistochemistry images of brains of Trem2^{+/-} mice with CPZ-induced CNS demyelination treated with anti-TREM2 antibody 7E5 or isotype control antibody as diagrammed in FIG. 5A. An anti-dMBP antibody was used to determine the amount of myelin debris accumulation; DAPI was used to image DNA. FIG. 5C provides a quantification of the experiments shown in FIGS. 5A-5B. The bars indicate the percent of dMBP+ staining at the indicated timepoints, which was measured as the number of dMBP-positive pixels per mm². The data shown are the mean±standard deviation (s.d.), with P<0.05 considered statistically significant. The asterisks indicate statistical significance (“*”=p<0.05; “**”=p<0.01; “***”=p<0.001; “****”=p<0.0001). In FIGS. 5B-5C, WK 4=four weeks after CPZ feeding; WK 4+3D=3 days after CPZ withdrawal; WK 4+7D=7 days after CPZ withdrawal; WK 4+14D=14 days after CPZ withdrawal.

FIGS. 6A-6B show the results of in vivo experiments evaluating the effects of anti-TREM2 antibody 7E5 on oligodendrocyte precursor cell (OPC) recruitment to demyelination lesions in the brains of Trem2+/− mice with CPZ-induced CNS demyelination. FIG. 6A shows representative immunohistochemistry images of PDGFRa in the brains of Trem2+/− mice administered anti-TREM2 antibody 7E5 or isotype control antibody as diagrammed in FIG. 5A. The images shown in FIG. 6A are from brain samples taken 3 days (WK4+3D) and 7 days (WK 4+7D) after CPZ withdrawal. DAPI was used to image DNA. FIG. 6B provides a quantification of the experiment shown in FIG. 6A. The bars indicate the density of PDGFRα⁺ cells at the indicated time points, which was measured as the number of PDGFRα-positive cells per mm². The data shown are the mean±standard deviation (s.d.), with P<0.05 considered statistically significant. The asterisks indicate statistical significance (“*”=p<0.05; “**”=p<0.01; “***”=p<0.001; “****”=p<0.0001).

FIGS. 7A-7C show the results of in vivo experiments evaluating the effects of anti-TREM2 antibody 7E5 on OPC differentiation into mature oligodendrocytes (OLs) in the brains of Trem2^{+/-} mice with CPZ-induced CNS demyelination. FIG. 7A shows representative immunohistochemistry images of OLIG2 and CNPase in the brains of Trem2+/− mice administered anti-TREM2 antibody 7E5 or isotype control antibody as diagrammed in FIG. 5A. The images shown in FIG. 7A are from brain samples taken 3 days after CPZ withdrawal (WK4+3D). DAPI was used to image DNA. FIGS. 7B-7C provide quantifications of the experiment shown in FIG. 7A at 3 days (WK4+3D) and 7 days (WK 4+7D) after CPZ withdrawal. In FIG. 7B, the bars indicate the density of OLIG2+ cells, which was measured as the number of OLIG2-positive cells per mm². In FIG. 7C, the bars indicate the percent of CNPase⁺ staining, which was measured as the number of CNPase-positive pixels per mm². In FIGS. 7B-7C, the data shown are the mean±standard deviation (s.d.), with P<0.05 considered statistically significant. The asterisks indicate statistical significance (“*”=p<0.05; “**”=p<0.01; “***”=p<0.001; “****”=p<0.0001).

FIGS. 8A-8B show the results of in vivo experiments evaluating the effects of anti-TREM2 antibody 7E5 on axonal health in the brains of Trem2+/− mice with CPZ-induced CNS demyelination. FIG. 8A shows representative immunohistochemistry images of SMI-31 in the brains of Trem2+/− mice administered anti-TREM2 antibody 7E5 or isotype control antibody as diagrammed in FIG. 5A. The images shown in FIG. 8A are from brain samples taken 7 days after CPZ withdrawal (WK 4+7D). FIG. 8B provides a quantification of the experiment shown in FIG. 8A at 3 days (WK4+3D) and 7 days (WK 4+7D) after CPZ withdrawal. The bars indicate the percent of SMI31+ staining at the indicated times, which was measured as the number of SMI31-positive pixels per mm². The data shown are the mean±standard deviation (s.d.), with P<0.05 considered statistically significant. The asterisks indicate statistical significance (“*”=p<0.05; “**”=p<0.01; “***”=p<0.001; “****”=p<0.0001).

### DETAILED DESCRIPTION OF THE PRESENT DISCLOSURE

Provided herein are methods of treating disorders and diseases associated with demyelination by administering an agonist of TREM2. Such diseases or disorders include, but are not limited to, multiple sclerosis, optic neuritis, neuromyelitis optica (Devic's disease), transverse myelitis, acute disseminated encephalomyelitis, adrenoleukodystrophy, and adrenomyeloneuropathy. Agonists of TREM2 include anti-TREM2 antibodies that induce one or more TREM2 activities, enhance one or more TREM2 activities induced in the presence of myelin, promote recruitment of oligodendrocyte precursor cells (OPCs) to one or more demyelination lesions in the CNS, promote an increase of mature oligodendrocytes (OLs) in one or more demyelination lesions in the CNS, promote differentiation of OPCs into mature oligodendrocytes (OLs) in one or more demyelination lesions in the CNS, promote an increase in the levels of phosphorylated neurofilaments in one or more demyelination lesions in the CNS, or promote clearance of myelin debris in one or more demyelination lesions in the individual.

### Definitions

As used herein, the term “preventing” includes providing prophylaxis with respect to occurrence or recurrence of a particular disease, disorder, or condition, including delaying onset of a particular disease, disorder, or condition, in an individual that may be predisposed to, susceptible to, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” antibody, such as an isolated anti-TREM2 antibody of the present disclosure, is one that has been identified, separated and/or recovered from a component of its production environment (e.g., naturally or recombinantly). Preferably, the isolated polypeptide is free from association with substantially all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain.

The “variable region” or “variable domain” of an antibody, such as an anti-TREM2 antibody of the present disclosure, refers to the amino-terminal domains of the heavy or light chain of the antibody. The variable domains of the heavy chain and light chain may be referred to as “V_{H}” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-TREM2 antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular cytotoxicity.

The term “monoclonal antibody” as used herein refers to an antibody, such as a monoclonal anti-TREM2 antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, monoclonal antibodies are advantageous in that they may be synthesized by a hybridoma culture, substantially uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), yeast presentation technologies (see, e.g., WO2009/036379A2; WO2010105256; WO2012009568, and Xu et al., Protein Eng. Des. Sel., 26(10): 663-70 (2013), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” or “whole antibody” are used interchangeably to refer to an antibody, such as an anti-TREM2 antibody of the present disclosure, in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-TREM2 antibodies of the present disclosure, produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments that are capable of binding and cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments may be produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which is recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) may have the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains, which enables the sFv to form the desired structure for antigen binding. For a review of the sFv, see Plückthun in The Pharmacology of Monoclonal Antibodies, vol. 113, Rosenburg and Moore eds., Springer-VerLAG-3, New York, pp. 269-315 (1994).

“Functional fragments” of antibodies, such as anti-TREM2 antibodies of the present disclosure, comprise a portion of an intact antibody, generally including the antigen binding or variable region of the intact antibody or the Fc region of an antibody which retains or has modified FcR binding capability.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the variable domains is achieved, thereby resulting in a bivalent fragment, i.e., a fragment having two antigen-binding sites. Diabodies are described in greater detail in, for example, EP 404,097; WO 93/11161; Hollinger et al., Proc. Nat'l Acad. Sci. USA 90:6444-48 (1993).

As used herein, a “chimeric antibody” refers to an antibody, such as a chimeric anti-TREM2 antibody of the present disclosure, in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies include antibodies in which the variable region of the antibody is derived from a murine antibody, and the constant region is derived from a human antibody. As used herein, “humanized antibody” is a subset of “chimeric antibodies.”

“Humanized” forms of non-human (e.g., murine) antibodies, such as humanized forms of anti-TREM2 antibodies of the present disclosure, are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody, such as an anti-TREM2 antibody of the present disclosure, that has been made using any of the techniques for making human antibodies as disclosed herein or otherwise known in the art. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology. Alternatively, human antibodies can also be prepared by employing yeast libraries and methods as disclosed in, for example, WO2009/036379A2; WO2010105256; WO2012009568; and Xu et al., Protein Eng. Des. Sel., 26(10): 663-70 (2013).

The term “hypervariable region” or “HVR” when used herein refers to the regions of an antibody-variable domain, such as that of an anti-TREM2 antibody of the present disclosure, that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the V_{H}. The variable domain residues are numbered according to Kabat et al., supra, for each of these extended HVR definitions.

“Framework” or “FR” residues are those variable domain residues other than the HVR residues as herein defined.

The phrase “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system,” “EU numbering” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. References to residue numbers in the variable domain of antibodies means residue numbering by the Kabat numbering system. References to residue numbers in the constant domain of antibodies means residue numbering by the EU numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferably those changes occur at only three, two, or one of positions 71H, 73H and 78H; for instance, the amino acid residues at those positions may by 71A, 73T and/or 78A. In one embodiment, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). For the VL, the subgroup may be, e.g., subgroup kappa I, kappa II, kappa III or kappa IV as in Kabat et al., supra. Additionally, for the VH, the subgroup may be, e.g., subgroup I, subgroup II, or subgroup III as in Kabat et al., supra.

An “amino acid modification” at a specified position, e.g., of an anti-TREM2 antibody of the present disclosure, refers to the substitution or deletion of the specified residue, or the insertion of at least one amino acid residue adjacent to the specified residue. Insertion “adjacent” to a specified residue means insertion within one to two residues thereof. The insertion may be N-terminal or C-terminal to the specified residue. The preferred amino acid modification herein is a substitution.

An “affinity matured” antibody, such as an affinity matured anti-TREM2 antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity matured antibodies may be produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As used herein, the term “specifically binds” refers to measurable and reproducible binding interactions between a target and an antibody, such as between an anti-TREM2 antibody and TREM2, that is determinative of the presence of the target within a heterogeneous population of molecules, such as biological molecules. For example, an antibody, such as an anti-TREM2 antibody of the present disclosure, that specifically binds to a target or an epitope of the target is an antibody that preferentially binds this target or epitope, e.g., with greater affinity or avidity, than it binds to other unrelated targets or epitopes. It is also understood that an antibody that specifically binds to a first target may or may not specifically bind to a second target. As such, “specific binding” does not necessarily require (although it can include) exclusive binding. An antibody that specifically binds to a target may have an association constant of at least about 10³M⁻¹ or 10⁴ M⁻¹, sometimes about 10⁵ M⁻¹ or 10⁶M⁻⁴, in other instances about 10⁶M⁻¹ or 10⁷ M⁻¹, about 10⁸ M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, or Vashist and Luong (2018) Handbook of Immunoassay Technologies, Approaches, Performances, and Applications, Academic Press, for a description of immunoassay formats and conditions that can be used to determine specific immunoreactivity.

As used herein, an antibody “inhibits interaction” between two proteins when the antibody disrupts, reduces, or completely eliminates an interaction between the two proteins by binding to one of the two proteins.

An “agonist” antibody is an antibody that induces (e.g., increases) one or more activities or functions of a target upon binding to the target.

An “antagonist” antibody or a “blocking” antibody is an antibody that reduces or eliminates (e.g., decreases) antigen binding to one or more binding partners after the antibody binds the antigen, and/or that reduces or eliminates (e.g., decreases) one or more activities or functions of the antigen after the antibody binds the antigen. In some embodiments, antagonist antibodies, or blocking antibodies substantially or completely inhibit antigen binding to one or more binding partners and/or one or more activities or functions of the antigen.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region, e.g. from about one to about ten amino acid substitutions, and preferably from about one to about five amino acid substitutions in a native sequence Fc region. The variant Fc region herein will preferably possess at least about 80% homology with a native sequence Fc region, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (see, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs are encompassed by the term “FcR” herein. FcRs can also increase the serum half-life of antibodies.

Binding to FcR in vivo and serum half-life of human FcR high-affinity binding polypeptides can be assayed, e.g., in transgenic mice or transfected human cell lines expressing human FcR, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

An “isolated” nucleic acid molecule, e.g., encoding an antibody such as an anti-TREM2 antibody of the present disclosure, is a nucleic acid molecule that is identified and separated from at least one contaminant nucleic acid molecule with which it is ordinarily associated in the environment in which it was produced. Preferably, the isolated nucleic acid is free of association with substantially all components associated with the production environment. The isolated nucleic acid molecules encoding the polypeptides and antibodies herein are distinguished from nucleic acid existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps”; substitution of one or more of the naturally occurring nucleotides with an analog; and internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “host cell” includes an individual cell or cell culture that can contain or contains a vector(s) or other exogenous nucleic acid, e.g., that incorporates a polynucleotide insert(s). In some embodiments, the vector or other exogenous nucleic acid is incorporated into the genome of the host cell. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### Overview

The present disclosure relates to methods of treating disorders and diseases associated with demyelination by administering an agonist of TREM2. Such diseases or disorders include, but are not limited to, multiple sclerosis, optic neuritis, neuromyelitis optica (Devic's disease), transverse myelitis, acute disseminated encephalomyelitis, adrenoleukodystrophy, and adrenomyeloneuropathy. Agonists of TREM2 include anti-TREM2 antibodies that induce one or more TREM2 activities and/or enhance one or more activities induced by binding of one or more ligands to TREM2. For example, agonist anti-TREM2 antibodies may decrease soluble TREM2, induce spleen tyrosine kinase (Syk) phosphorylation, induce binding of TREM2 to DAP12, induce DAP12 phosphorylation, increase the proliferation, survival, and/or function of dendritic cells, macrophages, monocytes, osteoclasts, Langerhans cells of skin, Kupffer cells, and microglial cells (microglia), or increase the activity and/or expression of TREM2-dependent genes.

Demyelination diseases, such as multiple sclerosis, are characterized by persistent demyelination and myelin debris accumulation within the brain, which eventually lead to axonal damage that clinically manifests as neurological disability (Dulamea 2017). Possible explanations for the impairment of remyelination in demyelination diseases include impaired oligodendrocyte precursor cell (OPC) activation or recruitment to the site of demyelination, and/or defects in OPC differentiation into myelinating mature oligodendrocytes (OLs) (Franklin and Ffrench-Constant 2017).

Accordingly, certain aspects of the present disclosure are based, at least in part, on the discovery, using an in vivo murine toxin-induced model of demyelination in the CNS, that an agonistic anti-TREM2 antibody (see, e.g., Example 2) significantly enhanced myelin debris clearance in vivo (see, e.g., Example 3); promoted recruitment of oligodendrocyte precursor cells (OPCs) to demyelination lesions in vivo (see, e.g., Example 4); promoted differentiation of OPCs into mature oligodendrocytes in vivo (see, e.g., Example 4); and promoted axonal health in the corpus callosum in vivo (see, e.g., Example 5).

### Methods of the Present Disclosure

The present disclosure provides methods of treating, preventing, or reducing risk of a central nervous system (CNS) demyelination disease, comprising administering to the individual in need thereof a therapeutically effective amount of an antibody that binds to a TREM2 protein, where the antibody is an agonist. In some embodiments, the antibody promotes remyelination in one or more demyelination lesions in the CNS of the individual.

Also provided herein are methods for promoting remyelination of one or more demyelination lesions in an individual having a central nervous system (CNS) demyelination disease, comprising administering to the individual a therapeutically effective amount of an antibody that binds to a TREM2 protein, where the antibody is an agonist.

### Demyelination Diseases

A “demyelination” or “demyelinating” disease, as used herein, refer to any disease, disorder or condition characterized by the presence of one or more demyelination lesions. Typically, demyelination lesions, also known as plaques, are characterized by damage to or loss of the myelin sheath of neurons in the white and/or gray matter of the central nervous system. In some cases, demyelination lesions exhibit T-cell and macrophage-mediated inflammatory reactions. In some embodiments, demyelination lesions may be diagnosed, measured, and/or classified according to known methods in the art, e.g., using the four demyelination lesion patterns described in Lucchinetti et al., (2000), Am Neurological Assoc, 47:707-17, and Popescu et al., (2013), Continuum (Minneap Minn.), 19 (4 Multiple Sclerosis): 901-21. Demyelination diseases include, without limitation, multiple sclerosis and others, such as optic neuritis, neuromyelitis optica (Devic's disease), transverse myelitis, acute disseminated encephalomyelitis, adrenoleukodystrophy, adrenomyeloneuropathy, mucopolysaccharidosis (Hurler syndrome), sphingolipidoses, Krabbe disease and other leukodystrophies, Leber hereditary optic atrophy, phenylketonuria, Tay-Sachs disease, Niemann-Pick disease, Gaucher disease, acute hemorrhagic leukoencephalitis, osmotic demyelination syndrome, and Marchiafava-Bignami disease. In some embodiments, the demyelination disease is multiple sclerosis.

Without wishing to be bound by theory, it is believed that agonizing TREM2 will ameliorate the symptoms of a demyelinating disease, e.g., multiple sclerosis. In certain embodiments, a TREM2 antibody as described herein may activate or increase signaling by TREM2 to compensate for or otherwise rescue reduced TREM2 function or copy number.

In some embodiments, an individual administered an anti-TREM2 antibody according to the methods provided herein has or is at risk for a disease characteristic selected from myelin damage, one or more demyelination lesions in the CNS, inflammation in the CNS, one or more plaques in the CNS, loss of myelin sheaths, axonal damage, reduced OPCs, reduced OLs, reduced myelin debris clearance, axonal varicosities, axonal spheroids, gliosis, autofluorescent lipid-laden macrophages, axon destruction, or any combination thereof. In some embodiments, the individual has or is at risk for having axonal damage and/or one or more demyelination lesions in the CNS. In some embodiments, the axonal damage and/or the one or more demyelination lesions in the CNS are in the white matter, the gray matter, or the corpus callosum of the CNS. In some embodiments, the individual has or is at risk for having a symptom selected from changes in sensation, pricking, numbness, muscle weakness, clonus, muscle spasms, difficulty in moving, difficulties with coordination, difficulties with balance, problems in speech, problems in swallowing, visual problems, fatigue, acute pain, chronic pain, bladder difficulties, bowel difficulties, cognitive impairment, depression, unstable mood, Uhthoffs phenomenon, Lhermitte's sign, or any combination thereof.

Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women.

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

In some embodiments, administering an anti-TREM2 antibody of the present disclosure can prevent, reduce the risk, and/or treat multiple sclerosis. In some embodiments, administering an anti-TREM2 antibody may induce one or more TREM2 activities in an individual having multiple sclerosis (e.g., DAP12 phosphorylation, PI3K activation, increased expression of one or more anti-inflammatory mediators, and reduced expression of one or more pro-inflammatory mediators). In some embodiments, administration of an anti-TREM2 antibody of the present disclosure results in an improvement in or a reduction of one or more symptoms of multiple sclerosis, including loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

### TREM2 Proteins

The present disclosure provides methods of treating, preventing, or reducing risk of a demyelinating disease in an individual comprising administering to the individual an antibody that binds to a TREM2 protein, wherein the antibody is an agonist.

Triggering receptor expressed on myeloid cells-2 (TREM2) is variously referred to as TREM-2, TREM2a, TREM2b, TREM2c, triggering receptor expressed on myeloid cells-2a, and triggering receptor expressed on monocytes-2. TREM2 is a 230 amino acid membrane protein. TREM2 is an immunoglobulin-like receptor primarily expressed on myeloid lineage cells, including without limitation, macrophages, dendritic cells, monocytes, Langerhans cells of skin, Kupffer cells, osteoclasts, and microglia. In some embodiments, TREM2 forms a receptor signaling complex with DAP12. In some embodiments, TREM2 phosphorylates and signals through DAP12 (an ITAM domain adaptor protein). In some embodiments, TREM2 signaling results in the downstream activation of PI3K or other intracellular signals. On myeloid cells, Toll-like receptor (TLR) signals are important for the activation of TREM2 activities, e.g., in the context of an infection response. TLRs also play a key role in the pathological inflammatory response, e.g., TLRs expressed in macrophages and dendritic cells.

TREM2 proteins of the present disclosure include, without limitation, a human TREM2 protein (Uniprot Accession No. Q9NZC2; SEQ ID NO: 1), and a non-human mammalian TREM2 protein, such as mouse TREM2 protein (Uniprot Accession No. Q99NH8; SEQ ID NO: 2), rat TREM2 protein (Uniprot Accession No. D3ZZ89; SEQ ID NO: 3), Rhesus monkey TREM2 protein (Uniprot Accession No. F6QVF2; SEQ ID NO: 4), cynomolgus monkey TREM2 protein (NCBI Accession No. XP_015304909.1; SEQ ID NO: 5), equine TREM2 protein (Uniprot Accession No. F7D6L0; SEQ ID NO: 6), pig TREM2 protein (Uniprot Accession No. H2EZZ3; SEQ ID NO: 7), and dog TREM2 protein (Uniprot Accession No. E2RP46; SEQ ID NO: 8). As used herein “TREM2 protein” refers to both wild-type sequences and naturally occurring variant sequences. In some embodiments, an agonist anti-TREM2 antibody of the disclosure binds to a wild-type TREM2 protein, a naturally occurring variant of a TREM2 protein, or a disease variant of a TREM2 protein.

In some embodiments, an example of a human TREM2 amino acid sequence is set forth below as SEQ ID NO: 1:

| | | | | | |
|---|---|---|---|---|---|
| 10 | 20 | 30 | 40 | 50 | 60 |
| MEPLRLLILL | FVTELSGAHN | TTVFQGVAGQ | SLQVSCPYDS | MKHWGRRKAW | CRQLGEKGPC |
| 70 | 80 | 90 | 100 | 110 | 120 |
| QRVVSTHNLW | LLSFLRRWNG | STAITDDTLG | GTLTITLRNL | QPHDAGLYQC | QSLHGSEADT |
| 130 | 140 | 150 | 160 | 170 | 180 |
| LRKVLVEVLA | DPLDHRDAGD | LWFPGESESF | EDAHVEHSIS | RSLLEGEIPF | PPTSILLLLA |
| 190 | 200 | 210 | 220 | 230 | |
| CIFLIKILAA | SALWAAAWHG | QKPGTHPPSE | LDCGHDPGYQ | LQTLPGLRDT | |

In some embodiments, the human TREM2 is a preprotein that includes a signal peptide. In some embodiments, the human TREM2 is a mature protein. In some embodiments, the mature TREM2 protein does not include a signal peptide. In some embodiments, the mature TREM2 protein is expressed on a cell. In some embodiments, TREM2 contains a signal peptide located at amino acid residues 1-18 of human TREM2 (SEQ ID NO: 1); an extracellular immunoglobulin-like variable-type (IgV) domain located at amino acid residues 29-112 of human TREM2 (SEQ ID NO: 1); additional extracellular sequences located at amino acid residues 113-174 of human TREM2 (SEQ ID NO: 1); a transmembrane domain located at amino acid residues 175-195 of human TREM2 (SEQ ID NO: 1); and an intracellular domain located at amino acid residues 196-230 of human TREM2 (SEQ ID NO: 1). The TREM2 cleavage site has been identified as occurring on the C-terminal side of Histidine 157 (see WO2018/015573), and cleavage at that site leads to shedding of the relevant portion of the TREM2 extracellular domain, detectable as an increase in soluble TREM2 (sTREM2) corresponding to that portion of TREM2.

The transmembrane domain of human TREM2 contains a lysine at amino acid residue 186 that can interact with an aspartic acid in DAP12, which is a key adaptor protein that transduces signaling from TREM2, TREM1, and other related IgV family members.

### TREM2 Mutations

In certain embodiments, a demyelinating disease involves a TREM2 protein with reduced function as compared to a TREM2 protein considered to have “wild type” function or that has function considered to be within normal range. In certain embodiments, a demyelinating disease is characterized by a mutation in a TREM2 gene in the affected individual. In certain embodiments, the mutation results in reduced function of TREM2 in the affected individual. The mutation may be of any type, including, for example, a missense mutation, an indel, or a mutation generating a truncated protein product. In some embodiments, the individual comprises at least one copy of a functional TREM2 gene. In some embodiments, the individual is heterozygous for a mutation in a TREM2 gene. In some embodiments, the individual is homozygous for a mutation in a TREM2 gene.

### Anti-TREM2 Antibodies

Certain aspects of the present disclosure relate to antibodies (e.g., monoclonal antibodies) that bind to a TREM2 protein, where the anti-TREM2 antibody is an agonist. In some embodiments, antibodies of the present disclosure bind a mature TREM2 protein. In some embodiments, antibodies of the present disclosure bind a mature TREM2 protein, wherein the mature TREM2 protein is expressed on a cell. In some embodiments, antibodies of the present disclosure bind a TREM2 protein expressed on one or more human cells selected from human dendritic cells, human macrophages, human monocytes, human osteoclasts, human Langerhans cells of skin, human Kupffer cells, human microglia, and any combinations thereof. In some embodiments, antibodies of the present disclosure bind a TREM2 protein expressed on one or more human microglia.

Anti-TREM2 Antibodies that Induce Activity and/or Enhance Ligand-Induced Activity

In some embodiments, an anti-TREM2 antibody of the present disclosure is an agonist antibody that induces one or more TREM2 activities. In some embodiments, the antibody induces one or more activities of TREM2 after binding to a TREM2 protein that is expressed on a cell.

In some embodiments, anti-TREM2 antibodies of the present disclosure bind to a TREM2 protein without competing with, inhibiting, or otherwise blocking one or more TREM2 ligands from binding to the TREM2 protein. Examples of TREM2 ligands include, without limitation, TREM2 ligands expressed by E. coli cells, apoptotic cells, nucleic acids, anionic lipids, APOE, APOE2, APOE3, APOE4, anionic APOE, anionic APOE2, anionic APOE3, anionic APOE4, lipidated APOE, lipidated APOE2, lipidated APOE3, lipidated APOE4, zwitterionic lipids, negatively charged phospholipids, phosphatidylserine, sulfatides, phosphatidylcholin, sphingomyelin, membrane phospholipids, lipidated proteins, proteolipids, lipidated peptides, and lipidated amyloid beta peptide. Accordingly, in certain embodiments, the one or more TREM2 ligands comprise E. coli cells, apoptotic cells, nucleic acids, anionic lipids, zwitterionic lipids, negatively charged phospholipids, phosphatidylserine (PS), sulfatides, phosphatidylcholin, sphingomyelin (SM), phospholipids, lipidated proteins, proteolipids, lipidated peptides, and lipidated amyloid beta peptide.

Anti-TREM2 antibodies used in the methods of the present disclosure are agonist antibodies. In some embodiments, antibodies of the present disclosure that bind a TREM2 protein may include agonist antibodies that due to their epitope specificity bind TREM2 and activate one or more TREM2 activities. In some embodiments, such antibodies may bind to the ligand-binding site on TREM2 and mimic the action of one or more TREM2 ligands, or stimulate TREM2 to transduce signal by binding to one or more domains that are not the ligand-binding sites. In some embodiments, the antibodies do not compete with or otherwise block ligand binding to TREM2. In some embodiments, the antibodies, act additively or synergistically with one or more TREM2 ligands to activate and/or enhance one more TREM2 activities, as set forth below.

Agonist anti-TREM2 antibodies of the present disclosure may display the ability to bind TREM2 without blocking simultaneous binding of one or more TREM2 ligands. The anti-TREM2 antibodies of the present disclosure may further display additive and/or synergistic functional interactions with one or more TREM2 ligands. Thus, in some embodiments, the maximal activity of TREM2 when bound to anti-TREM2 antibodies of the present disclosure in combination with one or more TREM2 ligands of the present disclosure may be greater (e.g., enhanced) than the maximal activity of TREM2 when exposed to saturating concentrations of ligand alone or to saturating concentrations of the antibody alone. In addition, the activity of TREM2 at a given concentration of TREM2 ligand may be greater (e.g., enhanced) in the presence of the antibody.

Accordingly, in some embodiments, anti-TREM2 antibodies of the present disclosure have an additive effect with the one or more TREM2 ligands to enhance the one or more TREM2 activities when bound to the TREM2 protein. In some embodiments, anti-TREM2 antibodies of the present disclosure synergize with the one or more TREM2 ligands to enhance the one or more TREM2 activities. In some embodiments, anti-TREM2 antibodies of the present disclosure increase the potency of the one or more TREM2 ligands to induce the one or more TREM2 activities, as compared to the potency of the one or more TREM2 ligands to induce the one or more TREM2 activities in the absence of the antibody. In some embodiments, anti-TREM2 antibodies of the present disclosure enhance the one or more TREM2 activities in the absence of cell surface clustering of TREM2. In some embodiments, anti-TREM2 antibodies of the present disclosure enhance the one or more TREM2 activities by inducing or retaining cell surface clustering of TREM2. In some embodiments, anti-TREM2 antibodies of the present disclosure are clustered by one or more Fc-gamma receptors expressed on one or more immune cells, including without limitation, B cells and microglial cells. In some embodiments, enhancement of the one or more TREM2 activities induced by binding of one or more TREM2 ligands to the TREM2 protein is measured on primary cells, including without limitation, dendritic cells, bone marrow-derived dendritic cells, monocytes, microglia, macrophages, neutrophils, NK cells, osteoclasts, Langerhans cells of skin, and Kupffer cells, or on cell lines.

In certain embodiments, an anti-TREM2 antibody of the present disclosure that enhances one or more TREM2 activities induced by binding of one or more TREM2 ligands to the TREM2 protein induces at least a 2-fold, at least a 3-fold, at least a 4-fold, at least a 5-fold, at least a 6-fold, at least a 7-fold, at least a 8-fold, at least a 9-fold, at least a 10-fold, at least an 11-fold, at least a 12-fold, at least a 13-fold, at least a 14-fold, at least a 15-fold, at least a 16-fold, at least a 17-fold, at least an 18-fold, at least a 19-fold, at least a 20-fold or greater increase in the one or more TREM2 activities as compared to levels of the one or more TREM2 activities induced by binding of the one or more TREM2 ligands to the TREM2 protein in the absence of the anti-TREM2 antibody.

In some embodiments, an agonist anti-TREM2 antibody of the present disclosure enhances one or more TREM2 activities induced in the presence of myelin. In some embodiments, the one or more TREM2 activities induced in the presence of myelin include increasing activity of one or more TREM2-dependent genes. In some embodiments, the one or more TREM2-dependent genes include nuclear factor of activated T-cells (NFAT) transcription factors. In some embodiments, the ability of anti-TREM2 antibodies to activate mouse or human TREM2-dependent genes is evaluated using a luciferase reporter gene under the control of an NFAT (nuclear factor of activated T-cells) promoter, e.g., as described below.

In some embodiments, TREM2 activities that may be induced and/or enhanced by anti-TREM2 antibodies of the present disclosure and/or one or more TREM2 ligands of the present disclosure include, without limitation, TREM2 binding to DAP12; DAP12 phosphorylation; activation of Syk kinase; modulation of one or more pro-inflammatory mediators selected from IFN-β, IL-1α, IL-1β, TNF-α, YM-1, IL-6, IL-8, CRP, CD86, MCP-1/CCL2, CCL3, CCL4, CCL5, CCR2, CXCL-10, Gata3, Rorc, IL-20 family members, IL-33, LIF, IFN-gamma, OSM, CNTF, GM-CSF, CSF-1, MHC-II, OPN, CD11c, GM-CSF, IL-11, IL-12, IL-17, IL-18, and IL-23, optionally where the modulation occurs in one or more cells selected from macrophages, M1 macrophages, activated M1 macrophages, M2 macrophages, dendritic cells, monocytes, osteoclasts, Langerhans cells of skin, Kupffer cells, and microglial cells; recruitment of Syk, ZAP70, or both to a DAP12/TREM2 complex; increasing activity of one or more TREM2-dependent genes, optionally where the one or more TREM2-dependent genes comprise nuclear factor of activated T-cells (NFAT) transcription factors; increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; modulated expression of one or more stimulatory molecules selected from CD83, CD86 MHC class II, CD40, and any combination thereof, optionally where the CD40 is expressed on dendritic cells, monocytes, macrophages, or any combination thereof, and optionally where the dendritic cells comprise bone marrow-derived dendritic cells; increasing memory; and reducing cognitive deficit. In some embodiments, anti-TREM2 antibodies of the present disclosure increase memory and/or reduce cognitive deficit when administered to an individual.

In some embodiments, an agonist anti-TREM2 antibody of the present disclosure induces one or more TREM2 activities selected from TREM2 binding to DAP12, DAP12 phosphorylation, activation of Syk kinase, recruitment of Syk to a DAP12/TREM2 complex, increasing activity of one or more TREM2-dependent genes, or any combination thereof. In some embodiments, the one or more TREM2-dependent genes include nuclear factor of activated T-cells (NFAT) transcription factors.

Syk Phosphorylation

In some embodiments, the anti-TREM2 antibodies of the present disclosure may induce spleen tyrosine kinase (Syk) phosphorylation after binding to a TREM2 protein expressed in a cell.

Spleen tyrosine kinase (Syk) is an intracellular signaling molecule that functions downstream of TREM2 by phosphorylating several substrates, thereby facilitating the formation of a signaling complex leading to cellular activation and inflammatory processes.

In some embodiments, the ability of agonist TREM2 antibodies to induce Syk activation is determined by culturing mouse macrophages and measuring the phosphorylation state of Syk protein in cell extracts. In some embodiments, bone marrow-derived macrophages (BMDM) from wild-type (WT) mice, from TREM2 knockout (KO) mice, and from mice that lack expression of functional Fc receptor common gamma chain gene (FcgR KO; REF: Takai T 1994. Cell 76(3):519-29) are starved for 4 hours in 1% serum RPMI and then removed from tissue culture dishes with PBS-EDTA, washed with PBS, and counted. In some embodiments, the cells are coated with full-length TREM2 antibodies, or with control antibodies for 15 minutes on ice. In some embodiments, after washing with cold PBS, cells are incubated at 37° C. for the indicated period of time in the presence of goat anti-human IgG. In some embodiments, after stimulation, cells are lysed with lysis buffer (1% v/v NP-40%, 50 Mm Tris-HCl (pH 8.0), 150 mM NaCl, 1 mM EDTA, 1.5 mM MgCl₂, 10% glycerol, plus protease and phosphatase inhibitors) followed by centrifugation at 16,000 g for 10 min at 4° C. to remove insoluble materials. In some embodiments, lysates are then immunoprecipitated with anti-Syk antibody (N-19 for BMDM or 4D10 for human DCs, Santa Cruz Biotechnology). In some embodiments, precipitated proteins are fractionated by SDS-PAGE, transferred to PVDF membranes and probed with anti-phosphotyrosine antibody (4G10, Millipore). In some embodiments, to confirm that all substrates are adequately immunoprecipitated, immunoblots are reprobed with anti-Syk antibody (Abcam, for BMDM) or anti-Syk (Novus Biological, for human DCs). In some embodiments, visualization is performed with the enhanced chemiluminescence (ECL) system (GE healthcare), as described (e.g., Peng et al., (2010) Sci Signal., 3(122): ra38).

DAP12 Binding and Phosphorylation

In some embodiments, the anti-TREM2 antibodies of the present disclosure may induce binding of TREM2 to DAP12. In other embodiments, the anti-TREM2 antibodies of the present disclosure may induce DAP12 phosphorylation after binding to a TREM2 protein expressed in a cell. In other embodiments, TREM2-mediated DAP12 phosphorylation is induced by one or more SRC family tyrosine kinases. Examples of Src family tyrosine kinases include, without limitation, Src, Syk, Yes, Fyn, Fgr, Lck, Hck, Blk, Lyn, and Frk.

DAP12 is variously referred to as TYRO protein tyrosine kinase-binding protein, TYROBP, KARAP, and PLOSL. DAP12 is a transmembrane signaling protein that contains an immunoreceptor tyrosine-based activation motif (ITAM) in its cytoplasmic domain. In certain embodiments, the anti-TREM2 antibody may induce DAP12 phosphorylation in its ITAM motif. Any method known in the art for determining protein phosphorylation, such as DAP12 phosphorylation, may be used.

In some embodiments, DAP12 is phosphorylated by SRC family kinases, resulting in the recruitment and activation of the Syk kinase, ZAP70 kinase, or both, to a DAP12/TREM2 complex.

In some embodiments, the ability of TREM2 antibodies to induce DAP12 activation is determined by culturing mouse macrophages and measuring the phosphorylation state of DAP12 protein in cell extracts. In some embodiments, before stimulation with antibodies, mouse wild-type (WT) bone marrow-derived macrophages (BMDM) and TREM2 knockout (KO) BMDM are starved for 4 h in 1% serum RPMI. In some embodiments, 15×10⁶ cells are incubated in ice for 15 min with full-length TREM2 antibodies or control antibodies. In some embodiments, cells are washed and incubated at 37° C. for the indicated period of time in the presence of goat anti-human IgG. In some embodiments, after stimulation, cells are lysed with lysis buffer (1% v/v n-Dodecyl-p-D-maltoside, 50 Mm Tris-HCl (pH 8.0), 150 mM NaCl, 1 mM EDTA, 1.5 mM MgCl₂, 10% glycerol, plus protease and phosphatase inhibitors), followed by centrifugation at 16,000 g for 10 min at 4° C. to remove insoluble materials. In some embodiments, cell lysate is immunoprecipitated with a second TREM2 antibody (R&D Systems). In some embodiments, precipitated proteins are fractionated by SDS-PAGE, transferred to PVDF membranes, and probed with anti-phosphotyrosine Ab (4G10, Millipore). In some embodiments, the membrane is stripped and reprobed with anti-DAP12 antibody (Cells Signaling, D7G1X). In some embodiments, each cell lysate used for TREM2 immunoprecipitations contains an equal amount of proteins, as indicated by a control antibody (anti-Actin, Santa Cruz).

Proliferation, Survival and Functionality of TREM2-Expressing Cells

In some embodiments, the anti-TREM2 antibodies of the present disclosure may increase the proliferation, survival, and/or function of dendritic cells, macrophages, monocytes, osteoclasts, Langerhans cells of skin, Kupffer cells, and microglial cells (microglia) after binding to TREM2 protein expressed in a cell. In some embodiments, the anti-TREM2 antibodies of the present disclosure do not inhibit the growth (e.g., proliferation and/or survival) of one or more innate immune cells.

In some embodiments, the anti-TREM2 antibodies of the present disclosure may increase the proliferation, survival, and/or function of microglial cells (microglia) after binding to TREM2 protein expressed in a cell. Microglial cells are a type of glial cell that are the resident macrophages of the brain and spinal cord, and thus act as the first and main form of active immune defense in the central nervous system (CNS). Microglial cells constitute 20% of the total glial cell population within the brain. Microglial cells are constantly scavenging the CNS for plaques, damaged neurons and infectious agents. The brain and spinal cord are considered “immune privileged” organs in that they are separated from the rest of the body by a series of endothelial cells known as the blood-brain barrier, which prevents most infections from reaching the vulnerable nervous tissue. In the case where infectious agents are directly introduced to the brain or cross the blood-brain barrier, microglial cells must react quickly to decrease inflammation and destroy the infectious agents before they damage the sensitive neural tissue. Due to the unavailability of antibodies from the rest of the body (few antibodies are small enough to cross the blood brain barrier), microglia must be able to recognize foreign bodies, swallow them, and act as antigen-presenting cells activating T-cells. Since this process must be done quickly to prevent potentially fatal damage, microglial cells are extremely sensitive to even small pathological changes in the CNS. They achieve this sensitivity in part by having unique potassium channels that respond to even small changes in extracellular potassium.

As used herein, macrophages of the present disclosure include, without limitation, M1 macrophages, activated M1 macrophages, and M2 macrophages. As used herein, microglial cells of the present disclosure include, without limitation, M1 microglial cells, activated M1 microglial cells, and M2 microglial cells.

In some embodiments, anti-TREM2 antibodies of the present disclosure may increase the expression of CD83 and/or CD86 on dendritic cells, monocytes, and/or macrophages.

As used herein, the rate of proliferation, survival, and/or function of macrophages, dendritic cells, monocytes, and/or microglia may include increased expression if the rate of proliferation, survival, and/or function of dendritic cells, macrophages, monocytes, osteoclasts, Langerhans cells of skin, Kupffer cells, and/or microglia in a subject treated with an anti-TREM2 antibody of the present disclosure is greater than the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody. In some embodiments, an anti-TREM2 antibody of the present disclosure may increase the rate of proliferation, survival, and/or function of dendritic cells, macrophages, monocytes, osteoclasts, Langerhans cells of skin, Kupffer cells, and/or microglia in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody. In other embodiments, an anti-TREM2 antibody of the present disclosure may increase the rate of proliferation, survival, and/or function of dendritic cells, macrophages, monocytes, osteoclasts, Langerhans cells of skin, Kupffer cells, and/or microglia in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody.

In some embodiments, to evaluate the ability of anti-TREM2 antibodies to induce or enhance cell survival in vitro, macrophages deficient in the gamma chain subunit of FcgRI, FcgRIII, and FceRI receptors (Fcgr1KO mice, REF: Takai T, Li M, Sylvestre D, Clynes R, Ravetch J. (1994). Cell, 76:519-529) are cultured in the presence of plate-bound anti-TREM2 antibodies and cell viability is determined when cells are cultured in suboptimal growth conditions. In some embodiments, murine bone marrow precursor cells from FcgR1 KO mice (Taconic, Model 584) are obtained by flushing tibial and femoral marrow cells with cold PBS. In some embodiments, after one wash with PBS, erythrocytes are lysed using ACK Lysing Buffer (Lonza), washed twice with PBS and suspended at 0.5×10⁶ cells/ml in complete RPMI media (10% FCS, Pen/Strep, Gln, neAA) with the indicated amount of M-CSF (Peprotech) to produce macrophages. In some embodiments, to analyze cell viability of bone marrow-derived macrophages, cells are prepared as above and plated at 2.5×10⁴/200 μl in a 96-well plate with suboptimal amounts of M-CSF (10 ng/ml) in non-tissue culture treated plates for two days. In some embodiments, cells are then quantified using the ToxGlo™ kit (Promega) and luminescence is determined as a measure of cell viability. In some embodiments, all experiments are conducted in the presence or absence of anti-TREM2 antibodies or isotype control antibodies.

TREM2-Dependent Gene Expression

In some embodiments, anti-TREM2 antibodies of the present disclosure may increase the activity and/or expression of TREM2-dependent genes, such as one or more transcription factors of the nuclear factor of activated T-cells (NFAT) family of transcription factors.

In some embodiments, the ability of soluble full-length anti-TREM2 antibodies to activate mouse or human TREM2-dependent genes is evaluated using a luciferase reporter gene under the control of an NFAT (nuclear factor of activated T-cells) promoter. In some embodiments, the cell line BW5147.G.1.4 (ATCC® TIB48™), derived from mouse thymus lymphoma T lymphocytes, is infected with mouse TREM2 and DAP12, and with Cignal Lenti NFAT-Luciferase virus (Qiagen). In some embodiments, alternatively the BW5147.G.1.4 cell line is infected with a human TREM2/DAP12 fusion protein, and with Cignal Lenti NFAT-Luciferase virus (Qiagen). In some embodiments, as a positive control for signaling, PMA (0.05 ug/ml) and ionomycin (0.25 uM) are added together. In some embodiments, cells are incubated together with soluble anti-TREM2 and isotype control antibodies for 6 hours and luciferase activity is measured by adding OneGlo Reagent (Promega) to each well and incubating 3 min at room temperature on a plate shaker. In some embodiments, luciferase signal is measured using a BioTek plate reader. In some embodiments, the cells display tonic TREM2-dependent signaling due to either the presence of an endogenous ligand or to spontaneous receptor aggregation, which leads to TREM2 signaling.

In some embodiments, the enhancement of the one or more TREM2 activities induced by binding of one or more TREM2 ligands to the TREM2 protein is measured, for example, utilizing an in vitro cell assay. In some embodiments, the increase in one or more TREM2 activities may be measured by any suitable in vitro cell-based assays or suitable in vivo model described herein or known in the art, for example, by utilizing a luciferase-based reporter assay to measure TREM2-dependent gene expression, using Western blot analysis to measure increase in TREM2-induced phosphorylation of downstream signaling partners, such as Syk, or by utilizing flow cytometry, such as fluorescence-activated cell sorting (FACS) to measure changes in cell surface levels of markers of TREM2 activation. Any in vitro cell-based assays or suitable in vivo model described herein or known in the art may be used to measure interaction (e.g., binding) between TREM2 and one or more TREM2 ligands.

In some embodiments, the increase in one or more TREM2 activities is measured by an in vitro cell-based assay. In some embodiments, to evaluate the ability of anti-TREM2 antibodies to enhance cell survival in vitro, macrophages deficient in the gamma chain subunit of FcgRI, FcgRIII, and FceRI receptors (Fcgr1KO mice, REF: Takai T, Li M, Sylvestre D, Clynes R, Ravetch J. (1994). Cell, 76:519-529) are cultured in the presence of plate-bound anti-TREM2 antibodies and cell viability is determined when cells are cultured in suboptimal growth conditions. In some embodiments, murine bone marrow precursor cells from FcgR1 KO mice (Taconic, Model 584) are obtained by flushing tibial and femoral marrow cells with cold PBS. In some embodiments, after one wash with PBS, erythrocytes are lysed using ACK Lysing Buffer (Lonza), washed twice with PBS and suspended at 0.5×10⁶ cells/ml in complete RPMI media (10% FCS, Pen/Strep, Gln, neAA) with the indicated amount of M-CSF (Peprotech) to produce macrophages. In some embodiments, to analyze cell viability of bone marrow-derived macrophages, cells are prepared as above and plated at 2.5×10⁴/200 μl in a 96-well plate with suboptimal amounts of M-CSF (10 ng/ml) in non-tissue culture treated plates for two days. In some embodiments, cells are then quantified using the ToxGlo™ kit (Promega) and luminescence is determined as a measure of cell viability. In some embodiments, all experiments are conducted in the presence or absence of anti-TREM2 antibodies or isotype control antibodies.

In some embodiments, the increase in one or more TREM2 activities is measured by an in vivo cell-based assay. In some embodiments, to evaluate the ability of anti-TREM2 antibodies to increase the number of immune cells in vivo, C57B16 mice are injected intraperitoneally (IP) with an anti-TREM2 antibody or a mouse IgG1 isotype control antibody, and the number of immune cells in the brain is quantified by FACS. In some embodiments, three to four mice per group receive an IP injection of 40 mg/kg anti-TREM2 antibody or isotype control antibody mIgG1 (clone MOPC-21, Bioxcell). In some embodiments, 48 hours later, the entire brains are harvested, rinsed with PBS, incubated at 37° C. in PBS containing 1 mg/ml collagenase and processed through a cell strainer to obtain a single cell suspension. In some embodiments, cells are then incubated with anti-CD45-PerCp-Cy7, anti-CD11b-PerCP-Cy5.5, anti-Gr1-FITC antibodies and a cell viability dye (Life Technologies, Cat #L34957) for 30 min on ice, then washed twice with cold FACS buffer. In some embodiments, 4% PFA-fixed samples are then analyzed by FACS. In some embodiments, data are acquired on a BD FACSCanto™ II cytometer (Becton Dickinson) and analyzed with FlowJo software.

In some embodiments, an anti-TREM2 antibody of the present disclosure enhances one or more TREM2 activities induced by binding of a TREM2 ligand to the TREM2 protein if it induces an increase that ranges from about 1.5-fold to about 6-fold, or more than 6-fold in ligand-induced TREM2-dependent gene transcription when used at a concentration that ranges from about 0.5 nM to about 50 nM, or greater than 50 nM, and as compared to the level of TREM2-dependent gene transcription induced by binding of the TREM2 ligand to the TREM2 protein in the absence of the anti-TREM2 antibody when the TREM2 ligand is used at its EC₅₀ concentration. In some embodiments, the increase in ligand-induced TREM2-dependent gene transcription is at least 1.5-fold, at least 2-fold, at least a 3-fold, at least a 4-fold, at least a 5-fold, at least a 6-fold, at least a 7-fold, at least a 8-fold, at least a 9-fold, at least a 10-fold, at least an 11-fold, at least a 12-fold, at least a 13-fold, at least a 14-fold, at least a 15-fold, at least a 16-fold, at least a 17-fold, at least an 18-fold, at least a 19-fold, at least a 20-fold or greater when used at a concentration that ranges from about 0.5 nM to about 50 nM, or greater than 50 nM, and as compared to the level of TREM2-dependent gene transcription induced by binding of the TREM2 ligand to the TREM2 protein in the absence of the anti-TREM2 antibody when the TREM2 ligand is used at its EC₅₀ concentration.

In some embodiments, the anti-TREM2 antibody is used at a concentration of at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, at least 10 nM, at least 15 nM, at least 20 nM, at least 25 nM, at least 30 nM, at least 35 nM, at least 40 nM, at least 45 nM, at least 46 nM, at least 47 nM, at least 48 nM, at least 49 nM, or at least 50 nM. In some embodiments, the TREM2 ligand is phosphatidylserine (PS). In some embodiments, the TREM2 ligand is sphingomyelin (SM). In some embodiments, the increase in one more TREM2 activities may be measured by any suitable in vitro cell-based assays or suitable in vivo model described herein or known in the art. In some embodiments, a luciferase-based reporter assay is used to measure the fold increase of ligand-induced TREM2-dependent gene expression in the presence and absence of antibody, as described in, for example, WO2017/062672 and WO2019/028292.

As used herein, an anti-TREM2 antibody of the present disclosure does not compete with, inhibit, or otherwise block the interaction (e.g., binding) between one or more TREM2 ligands and TREM2 if it decreases ligand binding to TREM2 by less than 20% at saturating antibody concentrations utilizing any in vitro assay or cell-based culture assay described herein or known in the art. In some embodiments, anti-TREM2 antibodies of the present disclosure inhibit interaction (e.g., binding) between one or more TREM2 ligands and TREM2 by less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% at saturating antibody concentrations utilizing any in vitro assay or cell-based culture assay described herein or known in the art.

Anti-TREM2 Antibodies that Decrease Soluble TREM2

In some embodiments, an agonist anti-TREM2 antibody decreases soluble TREM2 (sTREM2). In some embodiments, an agonist anti-TREM2 antibody decreases the level sTREM2 that is “shed” from the cell surface of a cell into an extracellular sample (e.g. shedding). In some embodiments, such an antibody binds to a region of TREM2 such that it blocks cleavage of TREM2. In such embodiments, the antibody binds to a region comprising His157, the cleavage site of TREM2.

The degree of inhibition of cleavage of TREM2 by an anti-TREM2 antibody negatively correlates with the amount of soluble TREM2 (sTREM2) in the presence of the anti-TREM2 antibody as compared to the amount of sTREM2 in the absence of the anti-TREM2 antibody. For example, an anti-TREM2 antibody may be considered as an anti-TREM2 antibody that inhibits cleavage of TREM2, if in the presence of said anti-TREM2 antibody the amount of sTREM2 is 0-90%, preferably 0-80%, more preferably 0-70%, even more preferably 0-60%, even more preferably 0-50% and even more preferable 0-20% of the amount of sTREM2 in the absence of the anti-TREM2 antibody, as assayed, e.g., by ELISA-based quantification of sTREM2.

In some embodiments, an anti-TREM2 antibody decreases levels of sTREM2 if the amount of sTREM2 in a treated sample is decreased by at least 10%, at least 20%, at least 30%, at least 40%, at least 50%, at least 60%, at least 70%, at least 80%, at least 90% or more as compared to a control value. In some embodiments, the control value is the amount of sTREM2 in an untreated sample (e.g., a supernatant from a TREM2-expressing cell that has not been treated with an anti-TREM2 antibody, or a sample from a subject that has not been treated with an anti-TREM2 antibody) or a sample treated with an appropriate non-TREM2-binding antibody.

In some embodiments, sTREM2 shedding is measured using a sample that comprises a fluid, e.g., blood, plasma, serum, urine, or cerebrospinal fluid. In some embodiments, the sample comprises cerebrospinal fluid. In some embodiments, the sample comprises supernatant from cell cultures (e.g., supernatant from a primary cell or cell line that endogenously expresses TREM2, such as human macrophages, or a primary cell or cell line that has been engineered to express TREM2.

In some embodiments, the level of sTREM2 in a sample is measured using an immunoassay. Immunoassays are known in the art and include, but are not limited to, enzyme immunoassays (EIA) such as enzyme multiplied immunoassay (EMIA), enzyme linked immunosorbent assay (ELISA), microparticle enzyme immunoassay (MEIA), immunohistochemistry (IHC), immunocytochemistry, capillary electrophoresis immunoassays (CEIA), radioimmunoassays (RIA), immunofluorescence, chemiluminescence immunoassays (CL), and electrochemiluminescence immunoassays (ECL). In some embodiments, sTREM2 levels are measuring using an ELISA assay.

In some embodiments, an ELISA assay can be used for quantitation of levels of sTREM2 in cell culture supernatants. In some embodiments, an ELISA for human sTREM2 is conducted using the Meso Scale Discovery SECTOR Imager 2400. In some embodiments, Streptavidin-coated 96-well plates are blocked overnight at 4° C. in 0.5% bovine serum albumin (BSA) and 0.05% Tween 20 in PBS (pH 7.4) (blocking buffer). In some embodiments, plates are shaken for 1 hour at room temperature with biotinylated polyclonal goat anti-human TREM2 capture antibody (0.25 mg/ml; R&D Systems) diluted in blocking buffer. In some embodiments, plates are washed subsequently four times with 0.05% Tween 20 in PBS (washing buffer) and incubated for 2 hours at room temperature with samples diluted 1:4 in 0.25% BSA and 0.05% Tween 20 in PBS (pH 7.4) (assay buffer) supplemented with protease inhibitors (Sigma). In some embodiments, recombinant human TREM2 protein (Holzel Diagnostika) is diluted in assay buffer in a two-fold serial dilution and used for the standard curve (concentration range, 4000 to 62.5 pg/ml). In some embodiments, plates are washed three times for 5 min with washing buffer before incubation for 1 hour at room temperature with mouse monoclonal anti-TREM2 antibody (1 mg/ml; Santa Cruz Biotechnology; B-3) diluted in blocking buffer. In some embodiments, after three additional washing steps, plates are incubated with a SULFO-TAG-labeled anti-mouse secondary antibody (1:1000; Meso Scale Discovery) and incubated for 1 hour in the dark. In some embodiments, plates are washed three times with washing buffer followed by two washing steps in PBS and developed by adding Meso Scale Discovery Read buffer. In some embodiments, the light emission at 620 nm after electrochemical stimulation is measured using the Meso Scale Discovery SECTOR Imager 2400 reader. In some embodiments, to quantify the levels of sTREM2 secreted, conditioned media from biological replicates are analyzed in duplicates. In some embodiments, sTREM2 standard curves are generated using the MasterPlex ReaderFit software (MiraiBio Group, Hitachi Solutions America) through a five-parameter logistic fit. In some embodiments, levels of sTREM2 are subsequently normalized to levels of immature TREM2 as quantified from Western Blots.

In some embodiments, sTREM2 may be inactive variants of cellular TREM2 receptors. In some embodiments, sTREM2 may be present in the periphery, such as in the plasma, or brains of subject, and may sequester anti-TREM2 antibodies. Such sequestered antibodies would be unable to bind to and activate, for example, the cellular TREM2 receptor present on cells. Accordingly, in certain embodiments, anti-TREM2 antibodies of the present disclosure, such as agonist anti-TREM2 antibodies of the present disclosure, do not bind to soluble TREM2. In some embodiments, anti-TREM2 antibodies of the present disclosure, such as agonist anti-TREM2 antibodies of the present disclosure, do not bind to soluble TREM2 in vivo. In some embodiments, agonist anti-TREM2 antibodies of the present disclosure that do not bind soluble TREM2 may bind to an epitope on TREM2 that, for example, may include a portion of the extracellular domain of cellular TREM2 that is not contained in sTREM2, for example one or more amino acid residues within amino acid residues 161-175; may be at or near a transmembrane portion of TREM2; or may include a transmembrane portion of TREM2.

Anti-TREM2 Antibodies that Promote Myelin Clearance

The pathological hallmark of demyelination diseases is the formation of demyelinating lesions in the CNS. For example, in multiple sclerosis, demyelination lesions may be found in the white and gray matter (Lassmann, Bruck et al. 2007). It is known that myelin debris removal and clearance by phagocytic cells are critical to eliminate inhibitory signals that can interfere with remyelination in the tissue (Kotter, Li et al. 2006; Lampron, Larochelle et al. 2015; Franklin and Ffrench-Constant 2017).

Accordingly, in some embodiments, the anti-TREM2 antibodies (e.g., agonist anti-TREM2 antibodies) of the present disclosure increase or promote the clearance of myelin debris in one or more demyelination lesions in an individual (e.g., in the CNS of an individual). For example, in some embodiments, an anti-TREM2 antibody of the disclosure reduces the amount of myelin debris in one or more demyelination lesions in an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In some embodiments, an anti-TREM2 antibody of the disclosure reduces the amount of myelin debris in one or more demyelination lesions in an individual compared to the amount of myelin debris in one or more demyelination lesions in the same individual prior to administration of the anti-TREM2 antibody. In some embodiments, an anti-TREM2 antibody of the disclosure reduces the amount of myelin debris in one or more demyelination lesions in an individual compared to the amount of myelin debris in one or more demyelination lesions in a corresponding individual that is not administered the anti-TREM2 antibody. In some embodiments, administration of an anti-TREM2 antibody of the disclosure to a plurality of individuals reduces the amount of myelin debris in one or more demyelination lesions in at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the individuals in the plurality compared to the amount of myelin debris in one or more demyelination lesions in the same plurality of individuals prior to administration of the anti-TREM2 antibody.

In some embodiments, an anti-TREM2 antibody of the disclosure increases the rate of myelin debris removal in one or more demyelination lesions by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold. In some embodiments, an anti-TREM2 antibody of the disclosure increases the rate of myelin debris removal in one or more demyelination lesions in an individual compared to the rate of myelin debris removal in one or more demyelination lesions in the same individual prior to administration of the anti-TREM2 antibody. In some embodiments, an anti-TREM2 antibody of the disclosure increases the rate of myelin debris removal in one or more demyelination lesions in an individual compared to the rate of myelin debris removal in a corresponding individual that is not administered the anti-TREM2 antibody. In some embodiments, administration of an anti-TREM2 antibody of the disclosure to a plurality of individuals increases the rate of myelin debris removal in one or more demyelination lesions in at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the individuals in the plurality compared to the rate of myelin debris removal in one or more demyelination lesions in the same plurality of individuals prior to administration of the anti-TREM2 antibody.

Demyelination lesions, and/or myelination status, may be measured in an individual using any method in the art, such as magnetization transfer ratio (MTR), restricted proton fraction f, myelin water fraction and diffusion tensor imaging (DTI), positron emission tomography (PET), and magnetic resonance imaging (MRI) (see, e.g., Mallik et al., (2014) J Neurology, Neurosurgery, and Psychiatry, 85(12):1396-1404).

Anti-TREM2 Antibodies that Promote Oligodendrocytes

Normally, oligodendrocyte (OL) loss during demyelination is followed by remyelination primarily sustained by oligodendrocyte precursor cells (OPCs), multipotent adult progenitor cells widely distributed in the CNS. In multiple sclerosis, impaired generation of OLs from OPCs leads to an imbalance between demyelination and remyelination, which is the main cause of persistent demyelination and myelin debris accumulation within the brain, eventually leading to axonal damage which clinically manifests as neurological disability (Dulamea 2017). Without wishing to be bound by theory, it is believed that failure of axon remyelination in demyelination diseases such as MS may be due to impaired OPC activation, impaired OPC recruitment to the site of demyelination, and/or defects in OPC differentiation into myelinating mature OLs (Franklin and Ffrench-Constant 2017).

Accordingly, in some embodiments, the anti-TREM2 antibodies (e.g., agonist anti-TREM2 antibodies) of the present disclosure increase or promote the recruitment of OPCs to one or more demyelination lesions in an individual (e.g., in the CNS of an individual). For example, in some embodiments, an anti-TREM2 antibody of the disclosure increases the numbers of OPCs in one or more demyelination lesions in an individual by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold. In some embodiments, an anti-TREM2 antibody of the disclosure increases the numbers of OPCs in one or more demyelination lesions in an individual compared to the numbers of OPCs in one or more demyelination lesions in the same individual prior to administration of the anti-TREM2 antibody. In some embodiments, an anti-TREM2 antibody of the disclosure increases the numbers of OPCs in one or more demyelination lesions in an individual compared to the numbers of OPCs in one or more demyelination lesions in a corresponding individual that is not administered the anti-TREM2 antibody. In some embodiments, administration of an anti-TREM2 antibody of the disclosure to a plurality of individuals increases the numbers of OPCs in one or more demyelination lesions in at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the individuals in the plurality compared to the numbers of OPCs in one or more demyelination lesions in the same plurality of individuals prior to administration of the anti-TREM2 antibody. In some embodiments, the OPCs are PDGFRa positive.

In other embodiments, the anti-TREM2 antibodies (e.g., agonist anti-TREM2 antibodies) of the present disclosure increase or promote the differentiation of OPCs into mature OLs in one or more demyelination lesions in an individual (e.g., in the CNS of an individual). For example, in some embodiments, an anti-TREM2 antibody of the disclosure increases the rate of differentiation of OPCs into mature OLs in one or more demyelination lesions by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold. In some embodiments, an anti-TREM2 antibody of the disclosure increases the rate of differentiation of OPCs into mature OLs in one or more demyelination lesions in an individual compared to the rate of differentiation of OPCs into mature OLs in one or more demyelination lesions in the same individual prior to administration of the anti-TREM2 antibody. In some embodiments, an anti-TREM2 antibody of the disclosure increases the rate of differentiation of OPCs into mature OLs in one or more demyelination lesions in an individual compared to the rate of differentiation of OPCs into mature OLs in one or more demyelination lesions in a corresponding individual that is not administered the anti-TREM2 antibody. In some embodiments, administration of an anti-TREM2 antibody of the disclosure to a plurality of individuals increases the rate of differentiation of OPCs into mature OLs in one or more demyelination lesions in at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the individuals in the plurality compared to the rate of differentiation of OPCs into mature OLs in one or more demyelination lesions in the same plurality of individuals prior to administration of the anti-TREM2 antibody.

In some embodiments, the anti-TREM2 antibodies (e.g., agonist anti-TREM2 antibodies) of the present disclosure promote an increase of mature OLs in one or more demyelination lesions in an individual (e.g., in the CNS of an individual). For example, in some embodiments, an anti-TREM2 antibody of the disclosure increases the numbers of mature OLs in one or more demyelination lesions in an individual by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold. In some embodiments, an anti-TREM2 antibody of the disclosure increases the numbers of mature OLs in one or more demyelination lesions in an individual compared to the numbers of mature OLs in one or more demyelination lesions in the same individual prior to administration of the anti-TREM2 antibody. In some embodiments, an anti-TREM2 antibody of the disclosure increases the numbers of mature OLs in one or more demyelination lesions in an individual compared to the numbers of mature OLs in one or more demyelination lesions in a corresponding individual that is not administered the anti-TREM2 antibody. In some embodiments, administration of an anti-TREM2 antibody of the disclosure to a plurality of individuals increases the numbers of mature OLs in one or more demyelination lesions in at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the individuals in the plurality compared to the numbers of mature OLs in one or more demyelination lesions in the same plurality of individuals prior to administration of the anti-TREM2 antibody.

In some embodiments, the mature OLs are OLIG2 and/or CNPase positive. Methods for quantifying mature OLs have been described (see, e.g., Lucchinetti et al. (1999) Brain 122(12):2279-2295).

Anti-TREM2 Antibodies that Promote Remyelination and Axonal Health

Demyelination in a demyelination disease such as multiple sclerosis eventually leads to axonal damage, which clinically manifests as neurological disability (Dulamea 2017).

Accordingly, in some embodiments, the anti-TREM2 antibodies (e.g., agonist anti-TREM2 antibodies) of the present disclosure increase or promote remyelination in one or more demyelination lesions in an individual (e.g., in the CNS of an individual). For example, in some embodiments, an anti-TREM2 antibody of the disclosure increases remyelination in one or more demyelination lesions in an individual by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold. In some embodiments, an anti-TREM2 antibody of the disclosure increases remyelination in one or more demyelination lesions in an individual compared to remyelination in one or more demyelination lesions in the same individual prior to administration of the anti-TREM2 antibody. In some embodiments, an anti-TREM2 antibody of the disclosure increases remyelination in one or more demyelination lesions in an individual compared to remyelination in one or more demyelination lesions in a corresponding individual that is not administered the anti-TREM2 antibody. In some embodiments, administration of an anti-TREM2 antibody of the disclosure to a plurality of individuals increases remyelination in one or more demyelination lesions in at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the individuals in the plurality compared to remyelination in one or more demyelination lesions in the same plurality of individuals prior to administration of the anti-TREM2 antibody.

In some embodiments, an anti-TREM2 antibody of the disclosure increases the rate of remyelination in one or more demyelination lesions by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold. In some embodiments, an anti-TREM2 antibody of the disclosure increases the rate of remyelination in one or more demyelination lesions in an individual compared to the rate of remyelination in one or more demyelination lesions in the same individual prior to administration of the anti-TREM2 antibody. In some embodiments, an anti-TREM2 antibody of the disclosure increases the rate of remyelination in one or more demyelination lesions in an individual compared to the rate of remyelination in one or more demyelination lesions in a corresponding individual that is not administered the anti-TREM2 antibody. In some embodiments, administration of an anti-TREM2 antibody of the disclosure to a plurality of individuals increases the rate of remyelination in one or more demyelination lesions in at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the individuals in the plurality compared to the rate of remyelination in one or more demyelination lesions in the same plurality of individuals prior to administration of the anti-TREM2 antibody.

In some embodiments, the anti-TREM2 antibodies (e.g., agonist anti-TREM2 antibodies) of the present disclosure increase or promote axonal health in one or more demyelination lesions in an individual (e.g., in the CNS of an individual). For example, in some embodiments, an anti-TREM2 antibody of the disclosure increases the levels of phosphorylated neurofilaments in one or more demyelination lesions in an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200%. In some embodiments, an anti-TREM2 antibody of the disclosure increases the levels of phosphorylated neurofilaments in one or more demyelination lesions in an individual compared to the levels of phosphorylated neurofilaments in one or more demyelination lesions in the same individual prior to administration of the anti-TREM2 antibody. In some embodiments, an anti-TREM2 antibody of the disclosure increases the levels of phosphorylated neurofilaments in one or more demyelination lesions in an individual compared to the levels of phosphorylated neurofilaments in one or more demyelination lesions in a corresponding individual that is not administered the anti-TREM2 antibody. In some embodiments, administration of an anti-TREM2 antibody of the disclosure to a plurality of individuals increases the levels of phosphorylated neurofilaments in one or more demyelination lesions in at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the individuals in the plurality compared to the levels of phosphorylated neurofilaments in one or more demyelination lesions in the same plurality of individuals prior to administration of the anti-TREM2 antibody. In some embodiments, the phosphorylated neurofilaments are SMI31 positive.

The level of remyelination, the rate of remyelination, and/or axonal health in one or more demyelination lesions may be measured in an individual using any method known in the art, such as magnetization transfer ratio (MTR), restricted proton fraction f, myelin water fraction and diffusion tensor imaging (DTI), positron emission tomography (PET), visual evoked potential (VEP) and magnetic resonance imaging (MRI) (see, e.g., Malik et al., (2014) J Neurology, Neurosurgery, and Psychiatry, 85(12):1396-1404; and Bove et al., (2017) Neurotherapeutics, 14:894-904).

In some embodiments, axonal health may be measured using biomarkers of axonal health and/or neurodegeneration. For example, biomarkers of axonal health and/or neurodegeneration (e.g., SIM31, SMI35, NF-L, NF-H, NF-M, alpha-Internexin, tau, amino acid n-acetyl aspartate, pNF-H, and other markers known in the art) may be detected in a sample of cerebrospinal fluid and/or a sample of blood from an individual using methods known in the art, such as ELISA, Western blots, mass spectrometry, and dot blots (see, e.g., Gresle et al., (2011) Multiple Sclerosis International, 315406).

Antibodies that Affect TREM2 Clustering

In vivo, anti-TREM2 antibodies of the present disclosure may activate receptors by multiple potential mechanisms. In some embodiments, agonistic anti-TREM2 antibodies of the present disclosure, have, due to appropriate epitope specificity, the ability to activate TREM2 in solution without having to be clustered with a secondary antibody, bound on plates, or clustered through Fcg receptors. In some embodiments, anti-TREM2 antibodies of the present disclosure have isotypes of human antibodies, such as IgG2, that have, due to their unique structure, an intrinsic ability to cluster receptors or retain receptors in a clustered configuration, thereby activating receptors such as TREM2 without binding to an Fc receptor (e.g., White et al., (2015) Cancer Cell 27, 138-148).

In certain embodiments, agonist anti-TREM2 antibodies may induce or maintain clustering on the cell surface in order to activate TREM2 and transduce a signal. In certain embodiments, agonist anti-TREM2 antibodies with appropriate epitope specificity may induce or maintain clustering of TREM2 on the cell surface and/or activate TREM2. In some embodiments, agonist anti-TREM2 antibodies bind to one or more amino acids within amino acid residues 124-153 of SEQ ID NO: 1, or amino acid residues on a TREM2 protein corresponding to amino acid residues 124-153 of SEQ ID NO: 1; within amino acid residues 129-153 of SEQ ID NO: 1, or amino acid residues on a TREM2 protein corresponding to amino acid residues 129-153 of SEQ ID NO: 1; within amino acid residues 140-149 of SEQ ID NO: 1, or amino acid residues on a TREM2 protein corresponding to amino acid residues 140-149 of SEQ ID NO: 1; within amino acid residues 149-157 of SEQ ID NO: 1, or amino acid residues on a TREM2 protein corresponding to amino acid residues 149-157 of SEQ ID NO: 1; or within amino acid residues 153-162 of SEQ ID NO: 1, or amino acid residues on a TREM2 protein corresponding to amino acid residues 153-162 of SEQ ID NO: 1. In some embodiments, agonist anti-TREM2 antibodies bind to one or more amino acid residues selected from the group consisting of D140, L141, W142, F143, P144, E151, D152, H154, E156, and H157 of SEQ ID NO: 1, or one or more amino acid residues on a mammalian TREM2 protein corresponding to an amino acid residue selected from the group consisting of D140, L141, W142, F143, P144, E151, D152, H154, E156, and H157 of SEQ ID NO: 1. In some embodiments, anti-TREM2 antibodies of the present disclosure cluster receptors (e.g., TREM2) by binding to Fcg receptors on adjacent cells. Binding of the constant IgG Fc part of the antibody to Fcg receptors leads to aggregation of the antibodies, and the antibodies in turn aggregate the receptors to which they bind through their variable region (Chu et al (2008) Mol Immunol, 45:3926-3933; and Wilson et al., (2011) Cancer Cell 19, 101-113). Any suitable assay known to one of skill in the art (such as those described in WO2017/062672 and WO2019/028292) may be used to determine antibody clustering.

Other mechanisms may also be used to cluster receptors (e.g., TREM2). For example, in some embodiments, antibody fragments (e.g., Fab fragments) that are cross-linked together may be used to cluster receptors (e.g., TREM2) in a manner similar to antibodies with Fc regions that bind Fcg receptors, as described above. In some embodiments, cross-linked antibody fragments (e.g., Fab fragments) may function as agonist antibodies if they induce receptor clustering on the cell surface and bind an appropriate epitope on the target (e.g., TREM2).

An antibody dependent on binding to FcgR receptor to activate targeted receptors may lose its agonist activity if engineered to eliminate FcgR binding (see, e.g., Wilson et al., (2011) Cancer Cell 19, 101-113; Armour at al., (2003) Immunology 40 (2003) 585-593); and White et al., (2015) Cancer Cell 27, 138-148). As such, it is thought that an anti-TREM2 antibody of the present disclosure with appropriate epitope specificity can activate TREM2 when the antibody has an Fc domain.

Exemplary antibody Fc isotypes and modifications are provided in Table A below. In some embodiments, the antibody has an Fc isotype listed in Table A below.

TABLE A
Exemplary antibody Fc isotypes that are capable of binding
Fc gamma receptor

| Fc Isotype | Mutation (EU numbering scheme) |
|---|---|
| IgG1 | N297A |
| IgG1 | D265A and N297A |
| IgG1 | D270A |
| IgG1 | L234A and L235A |
| | L234A and G237A |
| | L234A and L235A and G237A |
| IgG1 | P238D and/or L328E and/or S267E/L328F and/or E233 and |
| | or/G237D and/or H268D and/or P271G and/or A330R |
| IgG1 | P238D and L328E and E233D and G237D and H268D and |
| | P271Gand A330R |
| IgG1 | P238D and L328E and G237D and H268D and P271G and |
| | A330R |
| IgG1 | P238D and S267E and L328F and E233D and G237D and |
| | H268D and P271G and A330R |
| IgG1 | P238D and S267E and L328F and G237D and H268D and |
| | P271Gand A330R |
| IgG2 | V234A and G237A |
| IgG4 | L235A and G237A and E318A |
| IgG4 | S228P and L236E |
| IgG2/4 | IgG2 aa 118 to 260 and IgG4 aa 261 to 447 |
| hybrid | H268Q and V309L; and A330S and P331S |
| IgG1 | C226S and C229S and E233P and L234V and L235A |
| IgG1 | L234F and L235E and P331S |
| IgG2 | C232S or C233S |
| IgG2 | A330S and P331S |
| IgG1 | S267E, and L328F |
| | S267E alone |
| IgG2 | S267E and L328F |
| IgG4 | S267E and L328F |
| IgG2 | WT HC with Kappa (light chain) LC |
| | HC C127S with Kappa LC |
| | Kappa LC C214S |
| | Kappa LC C214S and HC C233S |
| | Kappa LC C214S and HC C232S |
| | Any of the above listed mutations together with A330S and |
| | P331S mutations |
| | F(ab’)2 fragment of WT IgGl and any of the above listed |
| | mutations |
| IgG1 | Substitute the Constant Heavy 1 (CHI) and hinge region of |
| | IgGl With CHI and hinge region of IGg2 |
| | ASTKGPSVFP LAPCSRSTSE STAALGCLVK |
| | DYFPEPVTVS WNSGALTSGV HTFPAVLQSS |
| | GLYSLSSVVT VPSSNFGTQT YTCNVDHKPS |
| | NTKVDKTVER KCCVECPPCP (SEQ ID NO: 42) |
| | With a Kappa LC |
| IgG1 | Any of the above listed mutations together with |
| | A330L/A330S and/ or L234F and/or L235E and/or P331S |
| IgG1, IgG2, | Any of the above listed mutations together with M252Y |
| or IgG4 | and/or S254T and/or T256E |
| Mouse IgG1 | For mouse disease models |
| IgG4 | WT |
| IgG1 | Any of the above listed mutation together with E430G, E430S, |
| | E430F, E430T, E345K, E345Q, E345R, E345Y, S440Y, |
| | S440W and/or any combination thereof. |
| IgG2 | Any of the above listed mutation together with E430G, E430S, |
| | E430F, E430T, E345K, E345Q, E345R, E345Y, S440Y, |
| | S440W and/or any combination thereof. |

In some embodiments, the antibody is of the IgG class, the IgM class, or the IgA class. In some embodiments, the antibody has an IgG1, IgG2, IgG3, or IgG4 isotype.

Antibodies with human IgG1 or IgG3 isotypes and mutants thereof (e.g. Strohl (2009) Current Opinion in Biotechnology 2009, 20:685-691) that bind the activating Fcg Receptors I, IIA, IIC, IIIA, IIIB in human and/or Fcg Receptors I, III and IV in mouse, may also act as agonist antibodies in vivo but may be associated with effects related to ADCC. However, such Fcg receptors appear to be less available for antibody binding in vivo, as compared to the inhibitory Fcg receptor FcgRIIB (see, e.g., White, et al., (2013) Cancer Immunol. Immunother. 62, 941-948; and Li et al., (2011) Science 333(6045):1030-1034).

In certain embodiments, the antibody has an IgG2 isotype. In some embodiments, the antibody contains a human IgG2 constant region. In some embodiments, the human IgG2 constant region includes an Fc region. In some embodiments, the antibody induces the one or more TREM2 activities, the DAP12 activities, or both independently of binding to an Fc receptor. In some embodiments, the antibody binds an inhibitory Fc receptor. In certain embodiments, the inhibitory Fc receptor is inhibitory Fc-gamma receptor IIB (FcγIIB), which minimizes or eliminates ADCC. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from V234A (Alegre et al., (1994) Transplantation 57:1537-1543. 31; Xu et al., (2000) Cell Immunol, 200:16-26), G237A (Cole et al. (1999) Transplantation, 68:563-571), H268Q, V309L, A330S, P331S (US 2007/0148167; Armour et al. (1999) Eur J Immunol 29: 2613-2624; Armour et al. (2000) The Haematology Journal 1 (Suppl.1):27; Armour et al. (2000) The Haematology Journal 1 (Suppl.1):27), C232S, and/or C233S (White et al., (2015) Cancer Cell 27, 138-148), S267E, L328F (Chu et al., (2008) Mol Immunol, 45:3926-3933), M252Y, S254T, and/or T256E, where the amino acid position is according to the EU numbering convention.

In some embodiments, the antibody has an IgG2 isotype with a heavy chain constant domain that contains a C127S amino acid substitution, where the amino acid position is according to the EU numbering convention (White et al., (2015) Cancer Cell 27, 138-148; Lightle et al., (2010) PROTEIN SCIENCE 19:753-762; and WO2008079246).

In some embodiments, the antibody has an IgG2 isotype with a Kappa light chain constant domain that contains a C214S amino acid substitution, where the amino acid position is according to the EU numbering convention (White et al., (2015) Cancer Cell 27, 138-148; Lightle et al., (2010) PROTEIN SCIENCE 19:753-762; and WO2008079246).

In certain embodiments, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor JIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some embodiments, the antibody includes an IgG2 isotype heavy chain constant domain 1 (CH1) and hinge region (White et al., (2015) Cancer Cell 27, 138-148). In certain embodiments, the IgG2 isotype CH1 and hinge region contain the amino acid sequence of ASTKGPSVFPLAPCSRSTSESTAALGCLVKDYFPEPVTVSWNSGALTSGVHTFPAVLQSSGLY SLSSVVTVPSSNFGTQTYTCNVDHKPSNTKVDKTVERKCCVECPPCP (SEQ ID NO: 42). In some embodiments, the antibody Fc region contains a S267E amino acid substitution, a L328F amino acid substitution, or both, and/or a N297A or N297Q amino acid substitution, where the amino acid position is according to the EU numbering convention.

In certain embodiments, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor JIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from L235A, G237A, S228P, L236E (Reddy et al., (2000) J Immunol, 164:1925-1933), S267E, E318A, L328F, M252Y, S254T, and/or T256E, where the amino acid position is according to the EU numbering convention.

In certain embodiments, the antibody has a hybrid IgG2/4 isotype. In some embodiments, the antibody includes an amino acid sequence containing amino acids 118 to 260 according to EU numbering of human IgG2 and amino acids 261-447 according to EU numbering of human IgG4 (WO 1997/11971; WO 2007/106585).

In certain embodiments, the antibody contains a mouse IgG4 constant region (Bartholomaeus, et al. (2014). J. Immunol. 192, 2091-2098).

In some embodiments, the Fc region further contains one or more additional amino acid substitutions selected from A330L, L234F; L235E, or P331S according to EU numbering; and any combination thereof.

In certain embodiments, the antibody contains one or more amino acid substitutions in the Fc region at a residue position selected from C127S, L234A, L234F, L235A, L235E, S267E, K322A, L328F, A330S, P331S, E345R, E430G, S440Y, and any combination thereof, where the numbering of the residues is according to EU numbering. In some embodiments, the Fc region contains an amino acid substitution at positions E430G, L243A, L235A, and P33iS, where the numbering of the residue position is according to EU numbering. In some embodiments, the Fc region contains an amino acid substitution at positions E430G and P331S, where the numbering of the residue position is according to EU numbering. In some embodiments, the Fc region contains an amino acid substitution at positions E430G and K322A, where the numbering of the residue position is according to EU numbering. In some embodiments, the Fc region contains an amino acid substitution at positions E430G, A330S, and P331S, where the numbering of the residue position is according to EU numbering. In some embodiments, the Fc region contains an amino acid substitution at positions E430G, K322A, A330S, and P331S, where the numbering of the residue position is according to EU numbering. In some embodiments, the Fc region contains an amino acid substitution at positions E430G, K322A, and A330S, where the numbering of the residue position is according to EU numbering. In some embodiments, the Fc region contains an amino acid substitution at positions E430G, K322A, and P331S, where the numbering of the residue position is according to EU numbering. In some embodiments, the Fc region contains an amino acid substitution at positions S267E and L328F, where the numbering of the residue position is according to EU numbering. In some embodiments, the Fc region contains an amino acid substitution at position C127S, where the numbering of the residue position is according to EU numbering. In some embodiments, the Fc region contains an amino acid substitution at positions E345R, E430G and S440Y, where the numbering of the residue position is according to EU numbering.

In some embodiments, the antibody has a human IgG1 isotype and comprises amino acid substitutions in the Fc region at the residue positions P331S and E430G, wherein the numbering of the residues is according to EU numbering. An Fc region comprising amino acid substitutions at the residue positions P331S and E430G may be referred to as “PSEG.”

Further IgG Mutations

In some embodiments, one or more of the IgG1 variants described herein may be combined with an A330L mutation (Lazar et al., (2006) Proc Natl Acad Sci USA, 103:4005-4010), or one or more of L234F, L235E, and/or P331S mutations (Sazinsky et al., (2008) Proc Natl Acad Sci USA, 105:20167-20172), where the amino acid position is according to the EU numbering convention, to eliminate complement activation. In some embodiments, the IgG variants described herein may be combined with one or more mutations to enhance the antibody half-life in human serum (e.g. M252Y, S254T, T256E mutations according to the EU numbering convention) (Dall'Acqua et al., (2006) J Biol Chem, 281:23514-23524; and Strohl e al., (2009) Current Opinion in Biotechnology, 20:685-691).

In some embodiments, an IgG4 variant of the present disclosure may be combined with an S228P mutation according to the EU numbering convention (Angal et al., (1993) Mol Immunol, 30:105-108) and/or with one or more mutations described in Peters et al., (2012) J Biol Chem. 13;287(29):24525-33) to enhance antibody stabilization.

Exemplary Anti-TREM2 Antibodies

In some embodiments, an anti-TREM2 antibody of the present disclosure binds to TREM2 with high affinity, is an agonist, and induces one or more TREM2 activities. In some embodiments, the anti-TREM2 antibody enhances one or more TREM2 activities induced by binding of one or more TREM2 ligands to the TREM2 protein, as compared to the one or more TREM2 activities induced by binding of the one or more TREM2 ligands to the TREM2 protein in the absence of the isolated antibody. In some embodiments, the anti-TREM2 antibody enhances the one or more TREM2 activities without competing with or otherwise blocking binding of the one or more TREM2 ligands to the TREM2 protein. In some embodiments, the antibody is a humanized antibody, a bispecific antibody, a multivalent antibody, or a chimeric antibody. Exemplary descriptions of such antibodies are found throughout the present disclosure. In some embodiments, the antibody is a bispecific antibody recognizing a first antigen and a second antigen.

In some embodiments, anti-TREM2 antibodies of the present disclosure bind to a human TREM2, or a homolog thereof, including without limitation a mammalian (e.g., non-human mammalian) TREM2 protein, mouse TREM2 protein (Uniprot Accession No. Q99NH8), rat TREM2 protein (Uniprot Accession No. D3ZZ89), Rhesus monkey TREM2 protein (Uniprot Accession No. F6QVF2), cynomolgus monkey TREM2 protein (NCBI Accession No. XP_015304909.1), equine TREM2 protein (Uniprot Accession No. F7D6L0), pig TREM2 protein (Uniprot Accession No. H2EZZ3), and dog TREM2 protein (Uniprot Accession No. E2RP46). In some embodiments, anti-TREM2 antibodies of the present disclosure specifically bind to human TREM2. In some embodiments, anti-TREM2 antibodies of the present disclosure specifically bind to cynomolgus monkey TREM2. In some embodiments, anti-TREM2 antibodies of the present disclosure specifically bind to both human TREM2 and cynomolgus monkey TREM2. In some embodiments, anti-TREM2 antibodies of the present disclosure induce at least one TREM2 activity of the present disclosure.

### Anti-TREM2 Antibody-Binding Regions

In some embodiments, anti-TREM2 antibodies of the present disclosure bind to one or more amino acids within amino acid residues 124-153 of SEQ ID NO: 1, or amino acid residues on a TREM2 protein corresponding to amino acid residues 124-153 of SEQ ID NO: 1; one or more amino acids within amino acid residues 129-153 of SEQ ID NO: 1, or amino acid residues on a TREM2 protein corresponding to amino acid residues 129-153 of SEQ ID NO: 1; one or more amino acids within amino acid residues 140-149 of SEQ ID NO: 1, or amino acid residues on a TREM2 protein corresponding to amino acid residues 140-149 of SEQ ID NO: 1; one or more amino acids within amino acid residues 149-157 of SEQ ID NO: 1, or amino acid residues on a TREM2 protein corresponding to amino acid residues 149-157 of SEQ ID NO: 1; or one or more amino acids within amino acid residues 153-162 of SEQ ID NO: 1, or amino acid residues on a TREM2 protein corresponding to amino acid residues 153-162 of SEQ ID NO: 1. In some embodiments, anti-TREM2 antibodies of the present disclosure bind one or more of amino acid residues D140, L141, W142, F143, P144, E151, D152, H154, E156, and H157 of SEQ ID NO: 1, or one or more amino acid residues on a mammalian TREM2 protein corresponding to an amino acid residue selected from the group consisting of D140, L141, W142, F143, P144, E151, D152, H154, E156, and H157 of SEQ ID NO: 1.

### Anti-TREM2 Antibody Light Chain and Heavy Chain Variable Regions

In some embodiments, anti-TREM2 antibodies to be used in the methods of the present disclosure are described in WO2018/195506, WO2019/028292, WO2018/015573, or WO2019/055841, each of which is hereby incorporated by reference herein. In some embodiments, the anti-TREM2 antibodies to be used in the methods of the present disclosure induce or enhance one or more of the following TREM2 activities: TREM2 binding to DAP12; DAP12 phosphorylation; activation of Syk kinase; modulation of one or more pro-inflammatory mediators selected from IFN-β, IL-1α, IL-1β, TNF-α, YM-1, IL-6, IL-8, CRP, CD86, MCP-1/CCL2, CCL3, CCL4, CCL5, CCR2, CXCL-10, Gata3, Rorc, IL-20 family members, IL-33, LIF, IFN-gamma, OSM, CNTF, GM-CSF, CSF-1, MHC-II, OPN, CD11c, GM-CSF, IL-11, IL-12, IL-17, IL-18, and IL-23, optionally where the modulation occurs in one or more cells selected from macrophages, M1 macrophages, activated M1 macrophages, M2 macrophages, dendritic cells, monocytes, osteoclasts, Langerhans cells of skin, Kupffer cells, and microglial cells; recruitment of Syk, ZAP70, or both to a DAP12/TREM2 complex; increasing activity of one or more TREM2-dependent genes, optionally where the one or more TREM2-dependent genes comprise nuclear factor of activated T-cells (NFAT) transcription factors; increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; modulated expression of one or more stimulatory molecules selected from CD83, CD86 MHC class II, CD40, and any combination thereof, optionally where the CD40 is expressed on dendritic cells, monocytes, macrophages, or any combination thereof, and optionally where the dendritic cells comprise bone marrow-derived dendritic cells; increasing memory; and reducing cognitive deficit. In some embodiments, anti-TREM2 antibodies of the present disclosure increase memory and/or reduce cognitive deficit when administered to an individual.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 34; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 35; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 31; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 41; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 33; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 32.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 36; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 37; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 38; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 39; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 40; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 32.

In some embodiments, anti-TREM2 antibodies of the present disclosure comprise a light chain variable domain and a heavy chain variable domain, wherein the heavy chain variable region comprises an HVR-H1 comprising the amino acid sequence YAFSSDWMN (SEQ ID NO: 36), an HVR-H2 comprising the amino acid sequence RIYPGEGDTNYARKFHG (SEQ ID NO: 37), an HVR-H3 comprising the amino acid sequence ARLLRNKPGESYAMDY (SEQ ID NO: 38), and the light chain variable region comprises an HVR-L1 comprising the amino acid sequence RTSQSLVHSNAYTYLH (SEQ ID NO: 39), an HVR-L2 comprising the amino acid sequence KVSNRVS (SEQ ID NO: 40), and an HVR-L3 comprising the amino acid sequence SQSTRVPYT (SEQ ID NO: 32). In some embodiments, anti-TREM2 antibodies of the present disclosure comprise a light chain variable domain and a heavy chain variable domain, wherein the heavy chain variable region comprises an HVR-H1 comprising the amino acid sequence YAFSSQWMN (SEQ ID NO: 34), an HVR-H2 comprising the amino acid sequence RIYPGGGDTNYAGKFQG (SEQ ID NO: 35), an HVR-H3 comprising the amino acid sequence ARLLRNQPGESYAMDY (SEQ ID NO: 31), and the light chain variable region comprises an HVR-L1 comprising the amino acid sequence RSSQSLVHSNRYTYLH (SEQ ID NO: 41), an HVR-L2 comprising the amino acid sequence KVSNRFS (SEQ ID NO: 33), and an HVR-L3 comprising the amino acid sequence SQSTRVPYT (SEQ ID NO: 32). In some embodiments, anti-TREM2 antibodies of the present disclosure comprise a heavy chain variable region and a light chain variable region, wherein the heavy chain variable region comprises one, two, three or four frame work regions selected from VH FR1, VH FR2, VH FR3, and VH FR4, wherein: the VH FRI comprises a sequence selected from the group consisting of SEQ ID NOs: 9-11, the VH FR2 comprises a sequence selected from the group consisting of SEQ ID NOs: 12 and 13, the VH FR3 comprises a sequence selected from the group consisting of SEQ ID NOs: 14 and 15, and the VH FR4 comprises the sequence of SEQ ID NO: 16; and/or the light chain variable region comprises one, two, three or four frame work regions selected from VL FRI, VL FR2, VL FR3, and VL FR4, wherein: the L FR1 comprises a sequence selected from the group consisting of SEQ ID NOs: 17-20, the VL FR2 comprises a sequence selected from the group consisting of SEQ ID NOs: 21 and 22, the VL FR3 comprises a sequence selected from the group consisting of SEQ ID NOs: 23 and 24, and the VL FR4 comprises a sequence selected from the group consisting of SEQ ID NOs: 25 and 26.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-47 or to the amino acid sequence of SEQ ID NO: 28; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-47 or to the amino acid sequence of SEQ ID NO: 29. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-47 or to the amino acid sequence of SEQ ID NO: 28, wherein the heavy chain variable domain comprises the HVR-H1, HVR-H2, and HVR-H3 amino acid sequences of antibody AL2p-47. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-47 or to the amino acid sequence of SEQ ID NO: 29, wherein the light chain variable domain comprises the HVR-L1, HVR-L2, and HVR-L3 amino acid sequences of antibody AL2p-47. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-47 or to the amino acid sequence of SEQ ID NO: 28 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody AL2p-47 or the amino acid sequence of SEQ ID NO: 28. In certain embodiments, a total of 1 to 5 amino acids have been substituted, inserted and/or deleted in the heavy chain variable domain amino acid sequence of antibody AL2p-47 or the amino acid sequence of SEQ ID NO: 28. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody AL2p-47 or of SEQ ID NO: 28, including post-translational modifications of that sequence. In a particular embodiment, the VH comprises one, two or three HVRs selected from: (a) the HVR-H1 amino acid sequence of antibody AL2p-47, (b) the HVR-H2 amino acid sequence of antibody AL2p-47, and (c) the HVR-H3 amino acid sequence of antibody AL2p-47. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-47 or to the amino acid sequence of SEQ ID NO: 29 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody AL2p-47 or the amino acid sequence of SEQ ID NO: 29. In certain embodiments, a total of 1 to 5 amino acids have been substituted, inserted and/or deleted in the light chain variable domain amino acid sequence of antibody AL2p-47 or the amino acid sequence of SEQ ID NO: 29. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody AL2p-47 or of SEQ ID NO: 29, including post-translational modifications of that sequence. In a particular embodiment, the VL comprises one, two or three HVRs selected from: (a) the HVR-L1 amino acid sequence of antibody AL2p-47, (b) the HVR-L2 amino acid sequence of antibody AL2p-47, and (c) the HVR-L3 amino acid sequence of antibody AL2p-47. In some embodiments, the antibody comprises a heavy chain variable region comprising the amino acid sequence of SEQ ID NO: 28 and a light chain variable region comprising the amino acid sequence of SEQ ID NO: 29.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-58 or to the amino acid sequence of SEQ ID NO: 27; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-58 or to the amino acid sequence of SEQ ID NO: 30. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-58 or to the amino acid sequence of SEQ ID NO: 27, wherein the heavy chain variable domain comprises the HVR-H1, HVR-H2, and HVR-H3 amino acid sequences of antibody AL2p-58. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-58 or to the amino acid sequence of SEQ ID NO: 30, wherein the light chain variable domain comprises the HVR-L1, HVR-L2, and HVR-L3 amino acid sequences of antibody AL2p-58. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-58 or to the amino acid sequence of SEQ ID NO: 27 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody AL2p-58 or the amino acid sequence of SEQ ID NO: 27. In certain embodiments, a total of 1 to 5 amino acids have been substituted, inserted and/or deleted in the heavy chain variable domain amino acid sequence of antibody AL2p-58 or the amino acid sequence of SEQ ID NO: 27. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody AL2p-58 or of SEQ ID NO: 27, including post-translational modifications of that sequence. In a particular embodiment, the VH comprises one, two or three HVRs selected from: (a) the HVR-H1 amino acid sequence of antibody AL2p-58, (b) the HVR-H2 amino acid sequence of antibody AL2p-58, and (c) the HVR-H3 amino acid sequence of antibody AL2p-58. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-58 or to the amino acid sequence of SEQ ID NO: 30 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody AL2p-58 or the amino acid sequence of SEQ ID NO: 30. In certain embodiments, a total of 1 to 5 amino acids have been substituted, inserted and/or deleted in the light chain variable domain amino acid sequence of antibody AL2p-58 or the amino acid sequence of SEQ ID NO: 30. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody AL2p-58 or of SEQ ID NO: 30, including post-translational modifications of that sequence. In a particular embodiment, the VL comprises one, two or three HVRs selected from: (a) the HVR-L1 amino acid sequence of antibody AL2p-58, (b) the HVR-L2 amino acid sequence of antibody AL2p-58, and (c) the HVR-L3 amino acid sequence of antibody AL2p-58. In some embodiments, the antibody comprises a heavy chain variable region comprising the amino acid sequence of SEQ ID NO: 27 and a light chain variable region comprising the amino acid sequence of SEQ ID NO: 30.

In some embodiments, the antibody comprises a heavy chain comprising the amino acid sequence of SEQ ID NO: 43, and a light chain comprising the amino acid sequence of SEQ ID NO: 47; or a heavy chain comprising the amino acid sequence of SEQ ID NO: 44, and a light chain comprising the amino acid sequence of SEQ ID NO: 47.

In some embodiments, the antibody comprises a heavy chain comprising the amino acid sequence of SEQ ID NO: 45, and a light chain comprising the amino acid sequence of SEQ ID NO: 48; or a heavy chain comprising the amino acid sequence of SEQ ID NO: 46, and a light chain comprising the amino acid sequence of SEQ ID NO: 48.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 52; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 53; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 54; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 55; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 56; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 57. In some embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 52; (b) an HVR-H2 comprising the amino acid sequence of SEQ ID NO: 53; and (c) an HVR-H3 comprising the amino acid sequence of SEQ ID NO: 54; and wherein the light chain variable domain comprises (a) an HVR-L1 comprising the amino acid sequence of SEQ ID NO: 55; (b) an HVR-L2 comprising the amino acid sequence of SEQ ID NO: 56; and (c) an HVR-L3 comprising the amino acid sequence of SEQ ID NO: 57.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 60; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 61; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 62; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 63; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 64; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 65. In some embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 60; (b) an HVR-H2 comprising the amino acid sequence of SEQ ID NO: 61; and (c) an HVR-H3 comprising the amino acid sequence of SEQ ID NO: 62; and wherein the light chain variable domain comprises: (a) an HVR-L1 comprising the amino acid sequence of SEQ ID NO: 63; (b) an HVR-L2 comprising the amino acid sequence of SEQ ID NO: 64; and (c) an HVR-L3 comprising the amino acid sequence of SEQ ID NO: 65.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 68; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 69; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 70; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 71; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 72; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 73. In some embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 68; (b) an HVR-H2 comprising the amino acid sequence of SEQ ID NO: 69; and (c) an HVR-H3 comprising the amino acid sequence of SEQ ID NO: 70; and wherein the light chain variable domain comprises: (a) an HVR-L1 comprising the amino acid sequence of SEQ ID NO: 71; (b) an HVR-L2 comprising the amino acid sequence of SEQ ID NO: 72; and (c) an HVR-L3 comprising the amino acid sequence of SEQ ID NO: 73.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 159; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 160; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 161; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 156; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 157; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 158. In some embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 159; (b) an HVR-H2 comprising the amino acid sequence of SEQ ID NO: 160; and (c) an HVR-H3 comprising the amino acid sequence of SEQ ID NO: 161; and wherein the light chain variable domain comprises: (a) an HVR-L1 comprising the amino acid sequence of SEQ ID NO: 156; (b) an HVR-L2 comprising the amino acid sequence of SEQ ID NO: 157; and (c) an HVR-L3 comprising the amino acid sequence of SEQ ID NO: 158. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 169; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 170; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 171; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 166; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 167; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 168. In some embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 169; (b) an HVR-H2 comprising the amino acid sequence of SEQ ID NO: 170; and (c) an HVR-H3 comprising the amino acid sequence of SEQ ID NO: 171; and wherein the light chain variable domain comprises: (a) an HVR-L1 comprising the amino acid sequence of SEQ ID NO: 166; (b) an HVR-L2 comprising the amino acid sequence of SEQ ID NO: 167; and (c) an HVR-L3 comprising the amino acid sequence of SEQ ID NO: 168. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 175; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 176; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 177; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 172; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 173; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 174. In some embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 175; (b) an HVR-H2 comprising the amino acid sequence of SEQ ID NO: 176; and (c) an HVR-H3 comprising the amino acid sequence of SEQ ID NO: 177; and wherein the light chain variable domain comprises: (a) an HVR-L1 comprising the amino acid sequence of SEQ ID NO: 172; (b) an HVR-L2 comprising the amino acid sequence of SEQ ID NO: 173; and (c) an HVR-L3 comprising the amino acid sequence of SEQ ID NO: 174. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 181; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 182; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 183; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 178; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 179; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 180. In some embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 181; (b) an HVR-H2 comprising the amino acid sequence of SEQ ID NO: 182; and (c) an HVR-H3 comprising the amino acid sequence of SEQ ID NO: 183; and wherein the light chain variable domain comprises: (a) an HVR-L1 comprising the amino acid sequence of SEQ ID NO: 178; (b) an HVR-L2 comprising the amino acid sequence of SEQ ID NO: 179; and (c) an HVR-L3 comprising the amino acid sequence of SEQ ID NO: 180. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 187; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 188; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 189; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 184; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 185; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 186. In some embodiments, anti-TREM2 antibodies of the present disclosure comprise a light chain variable domain and a heavy chain variable domain, wherein the heavy chain variable domain comprises: (a) an HVR-H1 comprising the amino acid sequence of SEQ ID NO: 187; (b) an HVR-H2 comprising the amino acid sequence of SEQ ID NO: 188; and (c) an HVR-H3 comprising the amino acid sequence of SEQ ID NO: 189; and wherein the light chain variable domain comprises: (a) an HVR-L1 comprising the amino acid sequence of SEQ ID NO: 184; (b) an HVR-L2 comprising the amino acid sequence of SEQ ID NO: 185; and (c) an HVR-L3 comprising the amino acid sequence of SEQ ID NO: 186. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 58; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 59. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 58, wherein the heavy chain variable domain comprises the HVR-H1, HVR-H2, and HVR-H3 amino acid sequences of antibody 42E8.H1. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 59, wherein the light chain variable domain comprises the HVR-L1, HVR-L2, and HVR-L3 amino acid sequences of antibody 42E8.H1. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 58 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 42E8.H1 or the amino acid sequence of SEQ ID NO: 58. In certain embodiments, a total of 1 to 5 amino acids have been substituted, inserted and/or deleted in the heavy chain variable domain amino acid sequence of antibody 42E8.H1 or the amino acid sequence of SEQ ID NO: 58. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody 42E8.H1 or of SEQ ID NO: 58, including post-translational modifications of that sequence. In a particular embodiment, the VH comprises one, two or three HVRs selected from: (a) the HVR-H1 amino acid sequence of antibody 42E8.H1, (b) the HVR-H2 amino acid sequence of antibody 42E8.H1, and (c) the HVR-H3 amino acid sequence of antibody 42E8.H1. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 59 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 42E8.H1 or the amino acid sequence of SEQ ID NO: 59. In certain embodiments, a total of 1 to 5 amino acids have been substituted, inserted and/or deleted in the light chain variable domain amino acid sequence of antibody 42E8.H1 or the amino acid sequence of SEQ ID NO: 59. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody 42E8.H1 or of SEQ ID NO: 59, including post-translational modifications of that sequence. In a particular embodiment, the VL comprises one, two or three HVRs selected from: (a) the HVR-L1 amino acid sequence of antibody 42E8.H1, (b) the HVR-L2 amino acid sequence of antibody 42E8.H1, and (c) the HVR-L3 amino acid sequence of antibody 42E8.H1. In some embodiments, the antibody comprises a heavy chain variable region comprising the amino acid sequence of SEQ ID NO: 58 and a light chain variable region comprising the amino acid sequence of SEQ ID NO: 59.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 66; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 67. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 66, wherein the heavy chain variable domain comprises the HVR-H1, HVR-H2, and HVR-H3 amino acid sequences of antibody RS9.F6. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 67, wherein the light chain variable domain comprises the HVR-L1, HVR-L2, and HVR-L3 amino acid sequences of antibody RS9.F6. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 66 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody RS9.F6 or the amino acid sequence of SEQ ID NO: 66. In certain embodiments, a total of 1 to 5 amino acids have been substituted, inserted and/or deleted in the heavy chain variable domain amino acid sequence of antibody RS9.F6 or the amino acid sequence of SEQ ID NO: 66. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody RS9.F6 or of SEQ ID NO: 66, including post-translational modifications of that sequence. In a particular embodiment, the VH comprises one, two or three HVRs selected from: (a) the HVR-H1 amino acid sequence of antibody RS9.F6, (b) the HVR-H2 amino acid sequence of antibody RS9.F6, and (c) the HVR-H3 amino acid sequence of antibody RS9.F6. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 67 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody RS9.F6 or the amino acid sequence of SEQ ID NO: 67. In certain embodiments, a total of 1 to 5 amino acids have been substituted, inserted and/or deleted in the light chain variable domain amino acid sequence of antibody RS9.F6 or the amino acid sequence of SEQ ID NO: 67. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody RS9.F6 or of SEQ ID NO: 67, including post-translational modifications of that sequence. In a particular embodiment, the VL comprises one, two or three HVRs selected from: (a) the HVR-L1 amino acid sequence of antibody RS9.F6, (b) the HVR-L2 amino acid sequence of antibody RS9.F6, and (c) the HVR-L3 amino acid sequence of antibody RS9.F6. In some embodiments, the antibody comprises a heavy chain variable region comprising the amino acid sequence of SEQ ID NO: 66 and a light chain variable region comprising the amino acid sequence of SEQ ID NO: 67.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 74; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 75. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 74 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the amino acid sequence of SEQ ID NO: 74. In certain embodiments, a total of 1 to 5 amino acids have been substituted, inserted and/or deleted in the amino acid sequence of SEQ ID NO: 74. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of SEQ ID NO: 74, including post-translational modifications of that sequence. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 75 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the amino acid sequence of SEQ ID NO: 75. In certain embodiments, a total of 1 to 5 amino acids have been substituted, inserted and/or deleted in the amino acid sequence of SEQ ID NO: 75. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of SEQ ID NO: 75, including post-translational modifications of that sequence. In some embodiments, the antibody comprises a heavy chain variable region comprising the amino acid sequence of SEQ ID NO: 74 and a light chain variable region comprising the amino acid sequence of SEQ ID NO: 75.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 6E7 or to the amino acid sequence of SEQ ID NO: 149; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 6E7 or to the amino acid sequence of SEQ ID NO: 148. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 6E7 or to the amino acid sequence of SEQ ID NO: 149, wherein the heavy chain variable domain comprises the HVR-H1, HVR-H2, and HVR-H3 amino acid sequences of antibody 6E7. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 6E7 or to the amino acid sequence of SEQ ID NO: 148, wherein the light chain variable domain comprises the HVR-L1, HVR-L2, and HVR-L3 amino acid sequences of antibody 6E7. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 6E7 or to the amino acid sequence of SEQ ID NO: 149 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 6E7 or the amino acid sequence of SEQ ID NO: 149. In certain embodiments, a total of 1 to 5 amino acids have been substituted, inserted and/or deleted in the heavy chain variable domain amino acid sequence of antibody 6E7 or the amino acid sequence of SEQ ID NO: 149. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 6E7 or of SEQ ID NO: 149, including post-translational modifications of that sequence. In a particular embodiment, the VH comprises one, two or three HVRs selected from: (a) the HVR-H1 amino acid sequence of antibody 6E7, (b) the HVR-H2 amino acid sequence of antibody 6E7, and (c) the HVR-H3 amino acid sequence of antibody 6E7. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 6E7 or to the amino acid sequence of SEQ ID NO: 148 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 6E7 or the amino acid sequence of SEQ ID NO: 148. In certain embodiments, a total of 1 to 5 amino acids have been substituted, inserted and/or deleted in the light chain variable domain amino acid sequence of antibody 6E7 or the amino acid sequence of SEQ ID NO: 148. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 6E7 or of SEQ ID NO: 148, including post-translational modifications of that sequence. In a particular embodiment, the VL comprises one, two or three HVRs selected from: (a) the HVR-L1 amino acid sequence of antibody 6E7, (b) the HVR-L2 amino acid sequence of antibody 6E7, and (c) the HVR-L3 amino acid sequence of antibody 6E7. In some embodiments, the antibody comprises a heavy chain variable region comprising the amino acid sequence of SEQ ID NO: 149 and a light chain variable region comprising the amino acid sequence of SEQ ID NO: 148. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 5E3 or to the amino acid sequence of SEQ ID NO: 151; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 5E3 or to the amino acid sequence of SEQ ID NO: 150. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 5E3 or to the amino acid sequence of SEQ ID NO: 151, wherein the heavy chain variable domain comprises the HVR-H1, HVR-H2, and HVR-H3 amino acid sequences of antibody 5E3. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 5E3 or to the amino acid sequence of SEQ ID NO: 150, wherein the light chain variable domain comprises the HVR-L1, HVR-L2, and HVR-L3 amino acid sequences of antibody 5E3. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 5E3 or to the amino acid sequence of SEQ ID NO: 151 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 5E3 or the amino acid sequence of SEQ ID NO: 151. In certain embodiments, a total of 1 to 5 amino acids have been substituted, inserted and/or deleted in the heavy chain variable domain amino acid sequence of antibody 5E3 or the amino acid sequence of SEQ ID NO: 151. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 5E3 or of SEQ ID NO: 151, including post-translational modifications of that sequence. In a particular embodiment, the VH comprises one, two or three HVRs selected from: (a) the HVR-H1 amino acid sequence of antibody 5E3, (b) the HVR-H2 amino acid sequence of antibody 5E3, and (c) the HVR-H3 amino acid sequence of antibody 5E3. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 5E3 or to the amino acid sequence of SEQ ID NO: 150 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 5E3 or the amino acid sequence of SEQ ID NO: 150. In certain embodiments, a total of 1 to 5 amino acids have been substituted, inserted and/or deleted in the light chain variable domain amino acid sequence of antibody 5E3 or the amino acid sequence of SEQ ID NO: 150. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 5E3 or of SEQ ID NO: 150, including post-translational modifications of that sequence. In a particular embodiment, the VL comprises one, two or three HVRs selected from: (a) the HVR-L1 amino acid sequence of antibody 5E3, (b) the HVR-L2 amino acid sequence of antibody 5E3, and (c) the HVR-L3 amino acid sequence of antibody 5E3. In some embodiments, the antibody comprises a heavy chain variable region comprising the amino acid sequence of SEQ ID NO: 151 and a light chain variable region comprising the amino acid sequence of SEQ ID NO: 150. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 24G6 or to the amino acid sequence of SEQ ID NO: 153; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 24G6 or to the amino acid sequence of SEQ ID NO: 152. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 24G6 or to the amino acid sequence of SEQ ID NO: 153, wherein the heavy chain variable domain comprises the HVR-H1, HVR-H2, and HVR-H3 amino acid sequences of antibody 24G6. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 24G6 or to the amino acid sequence of SEQ ID NO: 152, wherein the light chain variable domain comprises the HVR-L1, HVR-L2, and HVR-L3 amino acid sequences of antibody 24G6. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 24G6 or to the amino acid sequence of SEQ ID NO: 153 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 24G6 or the amino acid sequence of SEQ ID NO: 153. In certain embodiments, a total of 1 to 5 amino acids have been substituted, inserted and/or deleted in the heavy chain variable domain amino acid sequence of antibody 24G6 or the amino acid sequence of SEQ ID NO: 153. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 24G6 or of SEQ ID NO: 153, including post-translational modifications of that sequence. In a particular embodiment, the VH comprises one, two or three HVRs selected from: (a) the HVR-H1 amino acid sequence of antibody 24G6, (b) the HVR-H2 amino acid sequence of antibody 24G6, and (c) the HVR-H3 amino acid sequence of antibody 24G6. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 24G6 or to the amino acid sequence of SEQ ID NO: 152 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 24G6 or the amino acid sequence of SEQ ID NO: 152. In certain embodiments, a total of 1 to 5 amino acids have been substituted, inserted and/or deleted in the light chain variable domain amino acid sequence of antibody 24G6 or the amino acid sequence of SEQ ID NO: 152. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 24G6 or of SEQ ID NO: 152, including post-translational modifications of that sequence. In a particular embodiment, the VL comprises one, two or three HVRs selected from: (a) the HVR-L1 amino acid sequence of antibody 24G6, (b) the HVR-L2 amino acid sequence of antibody 24G6, and (c) the HVR-L3 amino acid sequence of antibody 24G6. In some embodiments, the antibody comprises a heavy chain variable region comprising the amino acid sequence of SEQ ID NO: 153 and a light chain variable region comprising the amino acid sequence of SEQ ID NO: 152. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 25F12 or to the amino acid sequence of SEQ ID NO: 155; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 25F12 or to the amino acid sequence of SEQ ID NO: 154. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 25F12 or to the amino acid sequence of SEQ ID NO: 155, wherein the heavy chain variable domain comprises the HVR-H1, HVR-H2, and HVR-H3 amino acid sequences of antibody 25F12. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 25F12 or to the amino acid sequence of SEQ ID NO: 154, wherein the light chain variable domain comprises the HVR-L1, HVR-L2, and HVR-L3 amino acid sequences of antibody 25F12. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 25F12 or to the amino acid sequence of SEQ ID NO: 155 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 25F12 or the amino acid sequence of SEQ ID NO: 155. In certain embodiments, a total of 1 to 5 amino acids have been substituted, inserted and/or deleted in the heavy chain variable domain amino acid sequence of antibody 25F12 or the amino acid sequence of SEQ ID NO: 155. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 25F12 or of SEQ ID NO: 155, including post-translational modifications of that sequence. In a particular embodiment, the VH comprises one, two or three HVRs selected from: (a) the HVR-H1 amino acid sequence of antibody 25F12, (b) the HVR-H2 amino acid sequence of antibody 25F12, and (c) the HVR-H3 amino acid sequence of antibody 25F12. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 25F12 or to the amino acid sequence of SEQ ID NO: 154 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 25F12 or the amino acid sequence of SEQ ID NO: 154. In certain embodiments, a total of 1 to 5 amino acids have been substituted, inserted and/or deleted in the light chain variable domain amino acid sequence of antibody 25F12 or the amino acid sequence of SEQ ID NO: 154. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 25F12 or of SEQ ID NO: 154, including post-translational modifications of that sequence. In a particular embodiment, the VL comprises one, two or three HVRs selected from: (a) the HVR-L1 amino acid sequence of antibody 25F12, (b) the HVR-L2 amino acid sequence of antibody 25F12, and (c) the HVR-L3 amino acid sequence of antibody 25F12. In some embodiments, the antibody comprises a heavy chain variable region comprising the amino acid sequence of SEQ ID NO: 155 and a light chain variable region comprising the amino acid sequence of SEQ ID NO: 154. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 14C12 or to the amino acid sequence of SEQ ID NO: 165; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 14C12 or to the amino acid sequence of SEQ ID NO: 164. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 14C12 or to the amino acid sequence of SEQ ID NO: 165, wherein the heavy chain variable domain comprises the HVR-H1, HVR-H2, and HVR-H3 amino acid sequences of antibody 13E7 14C12. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 14C12 or to the amino acid sequence of SEQ ID NO: 164, wherein the light chain variable domain comprises the HVR-L1, HVR-L2, and HVR-L3 amino acid sequences of antibody 13E7 14C12. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 14C12 or to the amino acid sequence of SEQ ID NO: 165 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 13E7 14C12 or the amino acid sequence of SEQ ID NO: 165. In certain embodiments, a total of 1 to 5 amino acids have been substituted, inserted and/or deleted in the heavy chain variable domain amino acid sequence of antibody 13E7 14C12 or the amino acid sequence of SEQ ID NO: 165. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 13E7 14C12 or of SEQ ID NO: 165, including post-translational modifications of that sequence. In a particular embodiment, the VH comprises one, two or three HVRs selected from: (a) the HVR-H1 amino acid sequence of antibody 13E7 14C12, (b) the HVR-H2 amino acid sequence of antibody 13E7 14C12, and (c) the HVR-H3 amino acid sequence of antibody 13E7 14C12. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 14C12 or to the amino acid sequence of SEQ ID NO: 164 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 13E7 14C12 or the amino acid sequence of SEQ ID NO: 164. In certain embodiments, a total of 1 to 5 amino acids have been substituted, inserted and/or deleted in the light chain variable domain amino acid sequence of antibody 13E7 14C12 or the amino acid sequence of SEQ ID NO: 164. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 13E7 14C12 or of SEQ ID NO: 164, including post-translational modifications of that sequence. In a particular embodiment, the VL comprises one, two or three HVRs selected from: (a) the HVR-L1 amino acid sequence of antibody 13E7 14C12, (b) the HVR-L2 amino acid sequence of antibody 13E7 14C12, and (c) the HVR-L3 amino acid sequence of antibody 13E7 14C12. In some embodiments, the antibody comprises a heavy chain variable region comprising the amino acid sequence of SEQ ID NO: 165 and a light chain variable region comprising the amino acid sequence of SEQ ID NO: 164. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 (SST202443) or to the amino acid sequence of SEQ ID NO: 163; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 (SST202443) or to the amino acid sequence of SEQ ID NO: 162. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 (SST202443) or to the amino acid sequence of SEQ ID NO: 163, wherein the heavy chain variable domain comprises the HVR-H1, HVR-H2, and HVR-H3 amino acid sequences of antibody 13E7 (SST202443). In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 (SST202443) or to the amino acid sequence of SEQ ID NO: 162, wherein the light chain variable domain comprises the HVR-L1, HVR-L2, and HVR-L3 amino acid sequences of antibody 13E7 (SST202443). In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 13E7 (SST202443) or to the amino acid sequence of SEQ ID NO: 163 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 13E7 (SST202443) or the amino acid sequence of SEQ ID NO: 163. In certain embodiments, a total of 1 to 5 amino acids have been substituted, inserted and/or deleted in the heavy chain variable domain amino acid sequence of antibody 13E7 (SST202443) or the amino acid sequence of SEQ ID NO: 163. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VH sequence of antibody 13E7 (SST202443) or of SEQ ID NO: 163, including post-translational modifications of that sequence. In a particular embodiment, the VH comprises one, two or three HVRs selected from: (a) the HVR-H1 amino acid sequence of antibody 13E7 (SST202443), (b) the HVR-H2 amino acid sequence of antibody 13E7 (SST202443), and (c) the HVR-H3 amino acid sequence of antibody 13E7 (SST202443). In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 13E7 (SST202443) or to the amino acid sequence of SEQ ID NO: 162 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 13E7 (SST202443) or the amino acid sequence of SEQ ID NO: 162. In certain embodiments, a total of 1 to 5 amino acids have been substituted, inserted and/or deleted in the light chain variable domain amino acid sequence of antibody 13E7 (SST202443) or the amino acid sequence of SEQ ID NO: 162. In certain embodiments, substitutions, insertions, or deletions occur in regions outside the HVRs (i.e., in the FR regions). In some embodiments, the substitutions, insertions, or deletions occur in the FR regions. Optionally, the anti-TREM2 antibody comprises the VL sequence of antibody 13E7 (SST202443) or of SEQ ID NO: 162, including post-translational modifications of that sequence. In a particular embodiment, the VL comprises one, two or three HVRs selected from: (a) the HVR-L1 amino acid sequence of antibody 13E7 (SST202443), (b) the HVR-L2 amino acid sequence of antibody 13E7 (SST202443), and (c) the HVR-L3 amino acid sequence of antibody 13E7 (SST202443). In some embodiments, the antibody comprises a heavy chain variable region comprising the amino acid sequence of SEQ ID NO: 163 and a light chain variable region comprising the amino acid sequence of SEQ ID NO: 162. In some embodiments, anti-TREM2 antibodies of the present disclosure compete with an antibody of any of the embodiments described in this paragraph for binding to TREM2. In some embodiments, anti-TREM2 antibodies of the present disclosure bind to the same TREM2 epitope as the TREM2 epitope bound by an antibody of any of the embodiments described in this paragraph.

In some embodiments, an agonist anti-TREM2 antibody of the present disclosure is AL2p-58 huIgG1 PSEG. In some embodiments, an agonist anti-TREM2 antibody of the present disclosure is AL2p-47 huIgG1. In the Examples provided herein, anti-TREM2 antibody 7E5 was used as an exemplary agonist anti-TREM2 antibody. Anti-TREM2 antibody 7E5 comprises a mouse IgG1 Fc region and binds to both mouse and human TREM2, making it suitable for use in the mouse cuprizone (CPZ) model described in the Examples. Anti-TREM2 antibody 7E5 possesses properties, including agonist activity, similar to agonist anti-TREM2 antibodies AL2p-58 huIgG1 PSEG and AL2p-47 huIgG1, as shown in the Examples herein and as disclosed in WO2017062672A2 and WO2019028292, which are hereby incorporated by reference in their entirety. Accordingly, anti-TREM2 antibody 7E5 is a suitable surrogate for anti-TREM2 antibodies AL2p-58 huIgG1 PSEG and AL2p-47 huIgG1.

TABLE B
Sequences

| SEQ | | |
|---|---|---|
| ID NO | Sequence | Description |
| 1 | MEPLRLLILLFVTELSGAHNTTVFQGVAGQSLQVSCPYDS | Human TREM2 |
| | MKHWGRRKAWCRQLGEKGPCQRVVSTHNLWLLSFLRR | protein |
| | WNGSTAITDDTLGGTLTITLRNLQPHDAGLYQCQSLHGSE | |
| | ADTLRKVLVEVLADPLDHRDAGDLWFPGESESFEDAHVE | |
| | HSISRSLLEGEIPFPPTSILLLLACIFLIKILAASALWAAAWH | |
| | GQKPGTHPPSELDCGHDPGYQLQTLPGLRDT | |
| 2 | MGPLHQFLLLLITALSQALNTTVLQGMAGQSLRVSCTYD | Mouse TREM2 |
| | ALKHWGRRKAWCRQLGEEGPCQRVVSTHGVWLLAFLK | protein |
| | KRNGSTVIADDTLAGTVTITLKNLQAGDAGLYQCQSLRG | |
| | REAEVLQKVLVEVLEDPLDDQDAGDLWVPEESSSFEGAQ | |
| | VEHSTSRNQETSFPPTSILLLLACVLLSKFLAASILWAVAR | |
| | GRQKPGTPVVRGLDCGQDAGHQLQILTGPGGT | |
| 3 | MEPLHVFVLLLVTELSQALNTTVLQGVAGQSLRVSCTYD | Rat TREM2 protein |
| | ALRHWGRRKAWCRQLAEEGPCQRVVSTHGVWLLAFLRK | |
| | QNGSTVITDDTLAGTVTITLRNLQAGDAGLYQCQSLRGRE | |
| | AEVLQKVVVEVLEDPLDDQDAGDLWVPEESESFEGAQVE | |
| | HSTSSQVSSCGSPLTYHLPPKEPIRKDLLPTHFHSSPPGLCP | |
| | PEQASYSQHPLGCGQGQAEAGDTCGQWARL | |
| 4 | MPDPLFSAVQGKDKILHKALCICPWPGKGGMEPLRLLILL | Rhesus monkey |
| | FATELSGAHNTTVFQGVEGQSLQVSCPYDSMKHWGRRK | TREM2 protein |
| | AWCRQLGEKGPCQRVVSTHNLWLLSFLRRRNGSTAITDD | |
| | TLGGTLTITLRNLQPHDAGFYQCQSLHGSEADTLRKVLVE | |
| | VLADPLDHRDAGDLWVPGESESFEDAHVEHSISRSLLEGE | |
| | IPFPPTSVLLLLACIFLIKILAASALWAAAWHGQKPGTHPPS | |
| | EPDCGHDPGHQLQTLPGLRDT | |
| 5 | MEPLRLLILLFATELSGAHNTTVFQGVEGQSLQVSCPYDS | Cynomolgus monkey |
| | MKHWGRRKAWCRQLGEKGPCQRVVSTHNLWLLSFLRRR | TREM2 protein |
| | NGSTAITDDTLGGTLTITLRNLQPHDAGFYQCQSLHGSEA | |
| | DTLRKVLVEVLADPLDHRDAGDLWVPGESESFEDAHVEH | |
| | SISRSLLEGEIPFPPTSVLLLLACIFLIKILAASALWAAAWH | |
| | GQKPGTHPPSEPDCGHDPGHQLQTLPGLRDT | |
| 6 | MEPLPLLILLSVAELSRGHNTTVFQGTAGRSLKVSCPYNSL | Equine TREM2 |
| | MHWGRRKAWCRQLGEDGPCQQWSTHSLWLLSFLKRRN | protein |
| | GSTVITDDALGGILTITLRNLQAHDAGFYQCQSLHGGEAD | |
| | TLRKVLVEVLADPLDHQEPGDLWIPKESESFEDAQVEHSIS | |
| | RSLVEEEIPSLPTSILLLLACIFLSKLLAASAIWAAAWHGQK | |
| | QETPPASEPDRGHDPGYQLHTLTGERDT | |
| 7 | METLGLLLLLWVAELSRAHNTSVFQGTAGQSLRVSCSYN | Pig TREM2 protein |
| | SLKHWGRRKAWCRQLSEEGLCQHVVSTHPTWLLSFLKRR | |
| | NGSTAITDDALGGTLTITLRNLQAHDAGLYQCQSLHGSEA | |
| | DTLKKVLVEVLADPLESQSKSFQDVQMEHSISRNLSEESLF | |
| | PPTSTLFLLACVFLSKLLVASALWAAAWHGHKQRTSPAG | |
| | GLDCGRDPGDQDQTLTDELGESSDQDQTLTELRDT | |
| 8 | MEPLWLLILLAVTELSGAHNTTVFQGMAGRSLQVSCPYN | Dog TREM2 protein |
| | SLKHWGRRKAWCRQVDKEGPCQRVVSTHRSWLLSFLKR | |
| | WNGSTAIVDDALGGTLTITLRNLQAHDAGLYQCQSLYGD | |
| | EADTLRKVLVEVLADPLDHLDPGDLWIPEESKGFEDAHV | |
| | EPSVSRSLSEEEIPFPPTSILFLLACIFLSKFLAASALWAAA | |
| | WRGQKLGTPQASELDCSCDPGYQLQTLTEPRDM | |
| 9 | QVQLVQSGAEVKKPGSSVKVSCKASG | VHFR1 |
| 10 | EVQLVQSGAEVKKPGSSVKVSCKASG | VHFR1 |
| 11 | QVQLVQSGAEVKKPGASVKVSCKASG | VHFR1 |
| 12 | WVRQAPGQGLEWMG | VHFR2 |
| 13 | WVRQAPGQRLEWIG | VHFR2 |
| 14 | RVTITADESTSTAYMELSSLRSEDTAVYYC | VHFR3 |
| 15 | RVTITADTSASTAYMELSSLRSEDTAVYYC | VHFR3 |
| 16 | WGQGTLVTVSS | VHFR4 |
| 17 | DVVMTQTPLSLSVTPGQPASISC | VLFR1 |
| 18 | GVVMTQTPLSLSVTPGQPASISC | VLFR1 |
| 19 | GVVMAQTPLSLSVTPGQPASISC | VLFR1 |
| 20 | DVVMTQSPDSLAVSLGERATINC | VLFR1 |
| 21 | WYLQKPGQSPQLLIY | VLFR2 |
| 22 | WYQQKPGQSPKLLIY | VLFR2 |
| 23 | GVPDRFSGSGSGTDFTLKISRVEAEDVGVYYC | VLFR3 |
| 24 | GVPDRFSGSGSGTDFTLTISSLQAEDVAVYYC | VLFR3 |
| 25 | FGQGTKLEIK | VLFR4 |
| 26 | FGGGTKVEIK | VLFR4 |
| 27 | QVQLVQSGAEVKKPGASVKVSCKASGYAFSSQWMNWV | AL2p-58-Heavy |
| | RQAPGQRLEWIGRIYPGGGDTNYAGKFQGRVTITADTSAS | chain variable |
| | TAYMELSSLRSEDTAVYYCARLLRNQPGESYAMDYWGQ | domain |
| | GTLVTVSS | |
| 28 | QVQLVQSGAEVKKPGSSVKVSCKASGYAFSSDWMNWVR | AL2p-47-Heavy |
| | QAPGQGLEWMGRIYPGEGDTNYARKFHGRVTITADESTS | chain variable |
| | TAYMELSSLRSEDTAVYYCARLLRNKPGESYAMDYWGQ | domain |
| | GTLVTVSS | |
| 29 | DVVMTQTPLSLSVTPGQPASISCRTSQSLVHSNAYTYLHW | AL2p-47-Light |
| | YLQKPGQSPQLLIYKVSNRVSGVPDRFSGSGSGTDFTLKIS | chain variable |
| | RVEAEDVGVYYCSQSTRVPYTFGQGTKLEIK | domain |
| 30 | DVVMTQSPDSLAVSLGERATINCRSSQSLVHSNRYTYLH | AL2p-58-Light |
| | WYQQKPGQSPKLLIYKVSNRFSGVPDRFSGSGSGTDFTLK | chain variable |
| | ISRVEAEDVGVYYCSQSTRVPYTFGQGTKLEIK | domain |
| 31 | ARLLRNQPGESYAMDY | AL2p-58-HVR-H3 |
| 32 | SQSTRVPYT | AL2p-58; |
| | | AL2p-47-HVR-L3 |
| 33 | KVSNRFS | AL2p-58-HVR-L2 |
| 34 | YAFSSQWMN | AL2p-58-HVR-H1 |
| 35 | RIYPGGGDTNYAGKFQG | AL2p-58-HVR-H2 |
| 36 | YAFSSDWMN | AL2p-47-HVR-H1 |
| 37 | RIYPGEGDTNYARKFHG | AL2p-47-HVR-H2 |
| 38 | ARLLRNKPGESYAMDY | AL2p-47-HVR-H3 |
| 39 | RTSQSLVHSNAYTYLH | AL2p-47-HVR-L1 |
| 40 | KVSNRVS | AL2p-47-HVR-L2 |
| 41 | RSSQSLVHSNRYTYLH | AL2p-58-HVR-L1 |
| 42 | ASTKGPSVFPLAPCSRSTSESTAALGCLVKDYFPEPVTVSW | IgG2 isotype heavy |
| | NSGALTSGVHTFPAVLQSSGLYSLSSVVTVPSSNFGTQTYT | chain constant |
| | CNVDHKPSNTKVDKTVERKCCVECPPCP | domain 1 (CH1) and |
| | | hinge region |
| 43 | QVQLVQSGAEVKKPGASVKVSCKASGYAFSSQWMNWV | AL2p-58 huIgG1 |
| | RQAPGQRLEWIGRIYPGGGDTNYAGKFQGRVTITADTSAS | PSEG-Heavy chain |
| | TAYMELSSLRSEDTAVYYCARLLRNQPGESYAMDYWGQ | |
| | GTLVTVSSASTKGPSVFPLAPSSKSTSGGTAALGCLVKDY | |
| | FPEPVTVSWNSGALTSGVHTFPAVLQSSGLYSLSSVVTVPS | |
| | SSLGTQTYICNVNHKPSNTKVDKKVEPKSCDKTHTCPPCP | |
| | APELLGGPSVFLFPPKPKDTLMISRTPEVTCVVVDVSHEDP | |
| | EVKFNWYVDGVEVHNAKTKPREEQYNSTYRVVSVLTVL | |
| | HQDWLNGKEYKCKVSNKALPASIEKTISKAKGQPREPQV | |
| | YTLPPSRDELTKNQVSLTCLVKGFYPSDIAVEWESNGQPE | |
| | NNYKTTPPVLDSDGSFFLYSKLTVDKSRWQQGNVFSCSV | |
| | MHGALHNHYTQKSLSLSPGK | |
| 44 | QVQLVQSGAEVKKPGASVKVSCKASGYAFSSQWMNWV | AL2p-58 huIgG1 |
| | RQAPGQRLEWIGRIYPGGGDTNYAGKFQGRVTITADTSAS | PSEG-Heavy chain |
| | TAYMELSSLRSEDTAVYYCARLLRNQPGESYAMDYWGQ | |
| | GTLVTVSSASTKGPSVFPLAPSSKSTSGGTAALGCLVKDY | |
| | FPEPVTVSWNSGALTSGVHTFPAVLQSSGLYSLSSVVTVPS | |
| | SSLGTQTYICNVNHKPSNTKVDKKVEPKSCDKTHTCPPCP | |
| | APELLGGPSVFLFPPKPKDTLMISRTPEVTCVVVDVSHEDP | |
| | EVKFNWYVDGVEVHNAKTKPREEQYNSTYRVVSVLTVL | |
| | HQDWLNGKEYKCKVSNKALPASIEKTISKAKGQPREPQV | |
| | YTLPPSRDELTKNQVSLTCLVKGFYPSDIAVEWESNGQPE | |
| | NNYKTTPPVLDSDGSFFLYSKLTVDKSRWQQGNVFSCSV | |
| | MHGALHNHYTQKSLSLSPG | |
| 45 | QVQLVQSGAEVKKPGSSVKVSCKASGYAFSSDWMNWVR | AL2p-47 huIgG1- |
| | QAPGQGLEWMGRIYPGEGDTNYARKFHGRVTITADESTS | Heavy chain |
| | TAYMELSSLRSEDTAVYYCARLLRNKPGESYAMDYWGQ | |
| | GTLVTVSSASTKGPSVFPLAPSSKSTSGGTAALGCLVKDY | |
| | FPEPVTVSWNSGALTSGVHTFPAVLQSSGLYSLSSVVTVPS | |
| | SSLGTQTYICNVNHKPSNTKVDKKVEPKSCDKTHTCPPCP | |
| | APELLGGPSVFLFPPKPKDTLMISRTPEVTCVVVDVSHEDP | |
| | EVKFNWYVDGVEVHNAKTKPREEQYNSTYRVVSVLTVL | |
| | HQDWLNGKEYKCKVSNKALPAPIEKTISKAKGQPREPQV | |
| | YTLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSPGK | |
| 46 | QVQLVQSGAEVKKPGSSVKVSCKASGYAFSSDWMNWVR | AL2p-47 huIgG1- |
| | QAPGQGLEWMGRIYPGEGDTNYARKFHGRVTITADESTS | Heavy chain |
| | TAYMELSSLRSEDTAVYYCARLLRNKPGESYAMDYWGQ | |
| | GTLVTVSSASTKGPSVFPLAPSSKSTSGGTAALGCLVKDY | |
| | FPEPVTVSWNSGALTSGVHTFPAVLQSSGLYSLSSVVTVPS | |
| | SSLGTQTYICNVNHKPSNTKVDKKVEPKSCDKTHTCPPCP | |
| | APELLGGPSVFLFPPKPKDTLMISRTPEVTCVVVDVSHEDP | |
| | EVKFNWYVDGVEVHNAKTKPREEQYNSTYRVVSVLTVL | |
| | HQDWLNGKEYKCKVSNKALPAPIEKTISKAKGQPREPQV | |
| | YTLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSPG | |
| 47 | DVVMTQSPDSLAVSLGERATINCRSSQSLVHSNRYTYLH | AL2p-58 huIgG1 |
| | WYQQKPGQSPKLLIYKVSNRFSGVPDRFSGSGSGTDFTLK | PSEG-Light chain |
| | ISRVEAEDVGVYYCSQSTRVPYTFGQGTKLEIKRTVAAPS | |
| | VFIFPPSDEQLKSGTASVVCLLNNFYPREAKVQWKVDNAL | |
| | QSGNSQESVTEQDSKDSTYSLSSTLTLSKADYEKHKVYAC | |
| | EVTHQGLSSPVTKSFNRGEC | |
| 48 | DVVMTQTPLSLSVTPGQPASISCRTSQSLVHSNAYTYLHW | AL2p-47 huIgG1- |
| | YLQKPGQSPQLLIYKVSNRVSGVPDRFSGSGSGTDFTLKIS | Light chain |
| | RVEAEDVGVYYCSQSTRVPYTFGQGTKLEIKRTVAAPSVF | |
| | IFPPSDEQLKSGTASVVCLLNNFYPREAKVQWKVDNALQS | |
| | GNSQESVTEQDSKDSTYSLSSTLTLSKADYEKHKVYACEV | |
| | THQGLSSPVTKSFNRGEC | |
| 49 | D/Ex0-2YxxL/IX6-8YxxL/I | Receptor motif |
| 52 | GYSITSDYAWN | 42E8.H1 CDR-H1 |
| 53 | YINYSGRTIYNPSLKS | 42E8.H1 CDR-H2 |
| 54 | ARWNGNYGFAY | 42E8.H1 CDR-H3 |
| 55 | RSSQSLVHINGNTYLH | 42E8.H1 CDR-L1 |
| 56 | KVSNRFS | 42E8.H1 CDR-L2 |
| 57 | SQTTHALFT | 42E8.H1 CDR-L3 |
| 58 | DVQLQESGPGLVKPSQSLSLTCTVTGYSITSDYAWNWIRQ | 42E8.H1 |
| | FPGNKLEWMGYINYSGRTIYNPSLKSRISITRDTSKNHFFL | Heavy Chain |
| | QLISVTTEDTATYYCARWNGNYGFAYWGQGTLVTVSA | Variable Region |
| 59 | DWMTQNPLSLPVSLGDQASISCRSSQSLVHINGNTYLHWY | 42E8.H1 |
| | LQKPGQSPKLLIYKVSNRFSGVPDRFSGSGSGTDFTLKISR | Light Chain Variable |
| | VEAEDLGVYFCSQTTHALFTFGSGTKLEIK | Region |
| 60 | GYTFTSY | RS9.F6 CDR-H1 |
| 61 | IGRSDPTTGGTNYNE | RS9.F6 CDR-H2 |
| 62 | VRTSGTGDY | RS9.F6 CDR-H3 |
| 63 | RSSQSLVHNNGNTFLH | RS9.F6 CDR-L1 |
| 64 | VSNRFS | RS9.F6 CDR-L2 |
| 65 | SQTTHVPPT | RS9.F6 CDR-L3 |
| 66 | QVQLQQPGAELVKPGASVKLSCKASGYTFTSYWMHWVK | RS9.F6 |
| | QSPGRGLEWIGRSDPTTGGTNYNEKFKTKATLTVDKPSST | Heavy Chain |
| | AYMQLSSLTSDDSAVYYCVRTSGTGDYWGQGTSLTVSSA | Variable Region |
| | KTTAPSVYPLAPVCGGTTGSSVT | |
| 67 | DVVMTQTPLSLPVSLGDQASISCRSSQSLVHNNGNTFLHW | RS9.F6 |
| | YLQKPGQSPKLLIYKVSNRFSGVPDRFSGSGSGTDFTLKIS | Light Chain Variable |
| | RVEAEDLGVYFCSQTTHVPPTFGGGTKLEIKRADAAPTVS | Region |
| | IFPPSSEQLTSGGASVVCF | |
| 68 | GFTFTDFY | WO2018/015573 |
| | | Consensus |
| | | CDR-H1 |
| 69 | IRNKANGYTT | WO2018/015573 |
| | | Consensus |
| | | CDR-H2 |
| 70 | ARIGINNGGSLDYWG | WO2018/015573 |
| | | Consensus |
| | | CDR-H3 |
| 71 | QSLLYSENNQDY | WO2018/015573 |
| | | Consensus |
| | | CDR-L1 |
| 72 | GAS | WO2018/015573 |
| | | Consensus |
| | | CDR-L2 |
| 73 | EQTYSYPYT | WO2018/015573 |
| | | Consensus |
| | | CDR-L3 |
| 74 | EVKLLESGGGLVQPGGSMRLSCAASGFTFTDFYMNWIRQ | WO2018/015573 |
| | PAGKAPEWLGLIRNKANGYTTEYNPSVKGRFTISRDNTQN | Consensus |
| | MLYLQMNTLR*EDTATYYCARIGINNGGSLDYWGQGVM | Heavy Chain |
| | VTVSS | Variable Region |
| | | The asterisk (*) in the |
| | | sequence can be any |
| | | amino acid. |
| 75 | DILINQSPASLTVSAGEKVTMSCKSSQSLLYSENNQDYLA | WO2018/015573 |
| | WYQQKPGQFPKLLIYGASNRHTGVPDRFTGSGSGTDFTLT | Consensus |
| | ISSVQAEDLADYYCEQTYSYPYTFGAGTKLELK | Light Chain Variable |
| | | Region |
| 76 | GFTFTDFY | WO2018/015573 |
| | | 14D3 |
| | | CDR-H1 |
| 77 | IRNKTKGYTT | WO2018/015573 |
| | | 14D3 |
| | | CDR-H2 |
| 78 | ARIGVNNGGSLDYWG | WO2018/015573 |
| | | 14D3 |
| | | CDR-H3 |
| 79 | QSLLYSENNQDY | WO2018/015573 |
| | | 14D3 |
| | | CDR-L1 |
| 80 | GAS | WO2018/015573 |
| | | 14D3 |
| | | CDR-L2 |
| 81 | EQTYSYPYT | WO2018/015573 |
| | | 14D3 |
| | | CDR-L3 |
| 82 | EVKLLEFGGGLVQPGGSMRLSCAASGFTFTDFYMNWIRQ | WO2018/015573 |
| | PAGRAPEWLGLIRNKTKGYTTEYNRSVKGRFTISRDNTQN | 14D3 |
| | MLYLQMNSLRPEDTATYYCARIGVNNGGSLDYWGQGVM | Heavy Chain |
| | VTVSS | Variable Region |
| 83 | DILIIQSPASLTVSAGARVTMSCKSSQSLLYSENNQDYLAW | WO2018/015573 |
| | YQQKPGQFPKLLIYGASNRHTGVPDRFTGSGSGTDFTLTIS | 14D3 |
| | SVQAEDLADYYCEQTYSYPYTFGAGTKLELK | Light Chain Variable |
| | | Region |
| 84 | GFTFTDFY | WO2018/015573 |
| | | 14D8 |
| | | CDR-H1 |
| 85 | IRNKANGYTT | WO2018/015573 |
| | | 14D8 |
| | | CDR-H2 |
| 86 | ARIGINNGGSLDYWG | WO2018/015573 |
| | | 14D8 |
| | | CDR-H3 |
| 87 | QSLLYSEKNQDY | WO2018/015573 |
| | | 14D8 |
| | | CDR-L1 |
| 88 | GAS | WO2018/015573 |
| | | 14D8 |
| | | CDR-L2 |
| 89 | EQTYSYPYT | WO2018/015573 |
| | | 14D8 |
| | | CDR-L3 |
| 90 | EVKLLESGGGLVQPGGSMRLSCAASGFTFTDFYMNWIRQ | WO2018/015573 |
| | PAGKAPEWLGLIRNKANGYTTVYNPSVKGRFTISRDNTQ | 14D8 |
| | NMLYLQMNTLRGEDTATYYCARIGINNGGSLDYWGQGV | Heavy Chain |
| | MVTVSS | Variable Region |
| 91 | DILINQSPASLTVSTGEKVTMSCRSSQSLLYSEKNQDYLA | WO2018/015573 |
| | WYQQKPGQFPKLLIYGASYRHTGVPDRFTGSGSGTDFTLT | 14D8 |
| | ISSVQAEDLADYYCEQTYSYPYTFGAGTKLELK | Light Chain Variable |
| | | Region |
| 92 | GFTFTDFY | WO2018/015573 |
| | | 7A12 |
| | | CDR-H1 |
| 93 | IRNKANGYTT | WO2018/015573 |
| | | 7A12 |
| | | CDR-H2 |
| 94 | ARIGINNGGSLDYWG | WO2018/015573 |
| | | 7A12 |
| | | CDR-H3 |
| 95 | QSLLYSEKNQDY | WO2018/015573 |
| | | 7A12 |
| | | CDR-L1 |
| 96 | GAS | WO2018/015573 |
| | | 7A12 |
| | | CDR-L2 |
| 97 | EQTYSYPYT | WO2018/015573 |
| | | 7A12 |
| | | CDR-L3 |
| 98 | EVKLLESGGGLVQPGGSMRLSCAASGFTFTDFYMNWIRQ | WO2018/015573 |
| | PAGKAPEWLGLIRNKANGYTTQYNPSVKGRFTISRDNTQ | 7A12 |
| | NMLYLQMNTLRGEDTATYYCARIGINNGGSLDYWGQGV | Heavy Chain |
| | MVTVSS | Variable Region |
| 99 | DILINQSPASLTVSAGEKVTMSCKSSQSLLYSEKNQDYLA | WO2018/015573 |
| | WYQQKPGQSPKLLMYGASYRHTGVPDRFTGSGSGTDFTL | 7A12 |
| | TISSVQAEDLADYYCEQTYSYPYTFGAGTKLELK | Light Chain Variable |
| | | Region |
| 100 | GFTFTDFY | WO2018/015573 |
| | | 8A11 |
| | | CDR-H1 |
| 101 | IRNKTKGYTT | WO2018/015573 |
| | | 8A11 |
| | | CDR-H2 |
| 102 | ARIGVNNGGSLDYWG | WO2018/015573 |
| | | 8A11 |
| | | CDR-H3 |
| 103 | QSLLYSENNQDY | WO2018/015573 |
| | | 8A11 |
| | | CDR-L1 |
| 104 | GAS | WO2018/015573 |
| | | 8A11 |
| | | CDR-L2 |
| 105 | EQTYSYPYT | WO2018/015573 |
| | | 8A11 |
| | | CDR-L3 |
| 106 | EVKLLESGGGLVQPGGSMRLSCAASGFTFTDFYMNWIRQ | WO2018/015573 |
| | PAGKAPEWLGLIRNKTKGYTTEYNTSVKGRFTISRDNTQN | 8A11 |
| | MLYLQMNSLRPEDTATYYCARIGVNNGGSLDYWGQGVM | Heavy Chain |
| | VTVSS | Variable Region |
| 107 | DILIIQSPASLTVSAGARVTMSCKSSQSLLYSENNQDYLAW | WO2018/015573 |
| | YQQKPGQFPKLLIYGASNRHTGVPDRFTGSGSGTDFTLTIS | 8A11 |
| | SVQAEDLADYYCEQTYSYPYTFGAGTKLELK | Light Chain Variable |
| | | Region |
| 108 | GFTFTDFY | WO2018/015573 |
| | | 21A3 |
| | | CDR-H1 |
| 109 | IRNKANGYTT | WO2018/015573 |
| | | 21A3 |
| | | CDR-H2 |
| 110 | ARIGINNGGSLDYWG | WO2018/015573 |
| | | 21A3 |
| | | CDR-H3 |
| 111 | QSLLYSEKNQDY | WO2018/015573 |
| | | 21A3 |
| | | CDR-L1 |
| 112 | GAS | WO2018/015573 |
| | | 21A3 |
| | | CDR-L2 |
| 113 | EQTYSYPYT | WO2018/015573 |
| | | 21A3 |
| | | CDR-L3 |
| 114 | EVKLLESGGGLVQPGGSMRLSCAASGFTFTDFYMNWIRQ | WO2018/015573 |
| | PAGKAPEWLGLIRNKANGYTTQYNPSVKGRFTISRDNTQ | 21A3 |
| | NMLYLQMNTLRGEDTATYYCARIGINNGGSLDYWGQGV | Heavy Chain |
| | MVTVSS | Variable Region |
| 115 | DILINQSPASLTVSAGEKVTMSCKSSQSLLYSEKNQDYLA | WO2018/015573 |
| | WYQQKPGQSPKLLMYGASYRHTGVPDRFTGSGSGTDFTL | 21A3 |
| | TISSVQAEDLADYYCEQTYSYPYTFGAGTKLELK | Light Chain Variable |
| | | Region |
| 116 | GFTFTDFY | WO2018/015573 |
| | | 10C3 |
| | | CDR-H1 |
| 117 | IRNKTKGYTT | WO2018/015573 |
| | | 10C3 |
| | | CDR-H2 |
| 118 | ARIGTNNGGSLDYWG | WO2018/015573 |
| | | 10C3 |
| | | CDR-H3 |
| 119 | QSLLYSENNQDY | WO2018/015573 |
| | | 10C3 |
| | | CDR-L1 |
| 120 | GAS | WO2018/015573 |
| | | 10C3 |
| | | CDR-L2 |
| 121 | EQTYSYPYT | WO2018/015573 |
| | | 10C3 |
| | | CDR-L3 |
| 122 | EVKLLESGGGLVQPGGSMRLSCAASGFTFTDFYMNWIRQ | WO2018/015573 |
| | PAGETPEWLGLIRNKTKGYTTEYNPSVKGRFTISRDNTQN | 10C3 |
| | MLYLQMNSLRPEDTATYYCARIGTNNGGSLDYWGQGVM | Heavy Chain |
| | VTVSS | Variable Region |
| 123 | DILIIQSPASLTVSAGARVTMSCKSSQSLLYSENNQDYLAW | WO2018/015573 |
| | YQQKPGQFPKLLIYGASNRHTGVPDRFTGSGSGTDFTLTIS | 10C3 |
| | SVQAEDLADYYCEQTYSYPYTFGAGTKLELK | Light Chain Variable |
| | | Region |
| 124 | GFTFTDFY | WO2018/015573 |
| | | 18F9 |
| | | CDR-H1 |
| 125 | IRNKVNGYRT | WO2018/015573 |
| | | 18F9 |
| | | CDR-H2 |
| 126 | ARIGINNGGSLDYWG | WO2018/015573 |
| | | 18F9 |
| | | CDR-H3 |
| 127 | QSLLYSENNQDY | WO2018/015573 |
| | | 18F9 |
| | | CDR-L1 |
| 128 | GAS | WO2018/015573 |
| | | 18F9 |
| | | CDR-L2 |
| 129 | EQTYSYPYT | WO2018/015573 |
| | | 18F9 |
| | | CDR-L3 |
| 130 | EVKLLESGGGLVQPGGSMRLSCVVSGFTFTDFYMNWIRQ | WO2018/015573 |
| | AAGKAPEWLGLIRNKVNGYRTEYNPSVKGRFTISRDNIQN | 18F9 |
| | MLYLQMNTLRAEDTATYYCARIGINNGGSLDYWGQGVM | Heavy Chain |
| | VTVSS | Variable Region |
| 131 | DILINQSPASLTVSAGEKVTMSCKSSQSLLYSENNQDYLA | WO2018/015573 |
| | WYQQKPGQFPKLLIYGASNRHTGVPDRFTGSGSGTDFTLT | 18F9 |
| | ISSVQAEDLADYYCEQTYSYPYTFGAGTKLELK | Light Chain Variable |
| | | Region |
| 132 | GFTFTDFY | WO2018/015573 |
| | | 15C5 |
| | | CDR-H1 |
| 133 | IRNKAYGYTT | WO2018/015573 |
| | | 15C5 |
| | | CDR-H2 |
| 134 | ARIGINYGGSLDYWG | WO2018/015573 |
| | | 15C5 |
| | | CDR-H3 |
| 135 | QSLLYSESNQDY | WO2018/015573 |
| | | 15C5 |
| | | CDR-L1 |
| 136 | GAS | WO2018/015573 |
| | | 15C5 |
| | | CDR-L2 |
| 137 | EQTYSYPYT | WO2018/015573 |
| | | 15C5 |
| | | CDR-L3 |
| 138 | EVKLLESGGGLVQPGGSMRLSCAASGFTFTDFYMNWIRQ | WO2018/015573 |
| | PAGKAPEWLGLIRNKAYGYTTEYNPSVKGRFTISRDNTQD | 15C5 |
| | MLYLQMNTLRAEDTATYYCARIGINYGGSLDYWGQGVM | Heavy Chain |
| | VTVSS | Variable Region |
| 139 | DILINQSPASLTVSAGEKVTVSCKSSQSLLYSESNQDYLAW | WO2018/015573 |
| | YQQKPGQFPKLLIYGASYRHTGVPDRFTGSGSGTDFTLTIS | 15C5 |
| | SVQAEDLAHYYCEQTYSYPYTFGAGTKLELK | Light Chain Variable |
| | | Region |
| 140 | GFTFTDFY | WO2018/015573 |
| | | 1G6 |
| | | CDR-H1 |
| 141 | IRNKANGFTT | WO2018/015573 |
| | | 1G6 |
| | | CDR-H2 |
| 142 | ARIGINNGGSLDYWG | WO2018/015573 |
| | | 1G6 |
| | | CDR-H3 |
| 143 | QSLLYSENKQDY | WO2018/015573 |
| | | 1G6 |
| | | CDR-L1 |
| 144 | GAS | WO2018/015573 |
| | | 1G6 |
| | | CDR-L2 |
| 145 | EQTYSYPYT | WO2018/015573 |
| | | 1G6 |
| | | CDR-L3 |
| 146 | EVKLLESGGGLVQPGGSLRLSCVASGFTFTDFYMNWIRQP | WO2018/015573 |
| | AGKAPEWLGLIRNKANGFTTEYNPSVKGRFTISRDNTQH | 1G6 |
| | MLYLQMNTLRAEDTATYYCARIGINNGGSLDYWGQGVM | Heavy Chain |
| | VTVSS | Variable Region |
| 147 | DILINQSPASLTVSTGEKVTMSCKSSQSLLYSENKQDYLA | WO2018/015573 |
| | WYQQKPGQFPKLLIYGASNRHTGVPDRFTGSGSGTDFTLT | 1G6 |
| | INIVQAEDLADYYCEQTYSYPYTFGAGTKLELK | Light Chain Variable |
| | | Region |
| 148 | DIQMTQSPSSVSASVGDRVTITCRASQGISSWLAWYQQKP | WO2018/195506 |
| | GKAPKLLIYAASSLQNGVPSRFSGSGSGTDFTLTISSLQPED | 6E7 |
| | FATYFCQQADSFPRTFGQGTKLEIK | Light Chain Variable |
| | | Region |
| 149 | EVQLVQSGAEVKKPGESLKISCKGSGYSFTSYWIAWVRQ | WO2018/195506 |
| | MPGKGLEWMGIIYPGDSDTRYSPSFQGQVTISADKSISTA | 6E7 |
| | YLQWSSLKASDTAMYFCARQRTFYYDSSDYFDYWGQG | Heavy Chain |
| | TLVTVSS | Variable Region |
| 150 | DIQMTQSPSSLSASVGDRVTITCRASQGISNYLAWFQQKP | WO2018/195506 |
| | GKAPKSLIYAASSLQSGVPSKFSGSGSGTDFTLTISSLQPE | 5E3 |
| | DFATYYCQQYSTYPFT FGPGTKVDIK | Light Chain Variable |
| | | Region |
| 151 | QVQLVQSGAEVKKPGASVKVSCKASGYTFTGYYIHWVR | WO2018/195506 |
| | QAPGLGLEWMGWINPYSGGTTSAQKFQGRVTMTRDTSIS | 5E3 |
| | SAYMELSRLRSDDTAVYYCARDGGYLALYGTDVWGQGT | Heavy Chain |
| | TVTVSS | Variable region |
| 152 | DIVMTQSPDSLAVSLGERATINCKSSQSVLYSSNNKHFLA | WO2018/195506 |
| | WYQQKPGQPPKLLIYWASTRESGVPDRFSGSGSGTDFTLT | 24G6 |
| | ISSLQAEDVAFYYCQQYYSTPLTFGGGTKVEIK | Light Chain Variable |
| | | Region |
| 153 | EVQLLESGGGLVQPGGSLRLSCAASGFTFSSYAMSWVRQ | WO2018/195506 |
| | APGKGLEWVSAISGSGGSTYYADSVKGRFTISRDNSKNTL | 24G6 |
| | YLQMNSLRAEDTAVYYCAKAYTPMAFFDYWGQGTLVTV | Heavy Chain |
| | SS | Variable Region |
| 154 | EKVMTQSPATLSVSPGERATLSCRASQSVNNNLAWYQQK | WO2018/195506 |
| | PGQAPRLLIYGASTRATGIPARFSGSGSGTEFTLTISSLQSE | 25F12 |
| | DFAVYYCQQYNNWPRTFGQGTKVEIK | Light Chain Variable |
| | | Region |
| 155 | QVQLQQWGAGLLKPSETLSLTCAVYGGSFSSYYWSWIRQ | WO2018/195506 |
| | PPGKGLEWIGEINHSGNTNYNPSLKSRVTISVDTSKNQFSL | 25F12 |
| | KLSSVTAADTAVYYCAREGYYDILTGYHDAFDIWDQGT | Heavy Chain |
| | MVTVFS | Variable Region |
| 156 | RASQSVSSNLA | WO2018/195506 |
| | | 13E7 14C12 |
| | | CDR-L1 |
| 157 | GASTRAT | WO2018/195506 |
| | | 13E7 14C12 |
| | | CDR-L2 |
| 158 | LQDNNWPPT | WO2018/195506 |
| | | 13E7 14C12 |
| | | CDR-L3 |
| 159 | SYWIG | WO2018/195506 |
| | | 13E7 14C12 |
| | | CDR-H1 |
| 160 | IIYPGDSDTRYSPSFQG | WO2018/195506 |
| | | 13E7 14C12 |
| | | CDR-H2 |
| 161 | RRQGIWGDALDF | WO2018/195506 |
| | | 13E7 14C12 |
| | | CDR-H3 |
| 162 | EIVMTQSPATLSVSPGERATLSCRASQSVSSNLAWFQQKP | WO2018/195506 |
| | GQAPRLLIYGASTRATGIPARFSGSGSGTEFTLTISSLQPED | 13E7 (SST202443) |
| | FAVYYCLQDNNFPPTFGQGTKVDIK | Light Chain Variable |
| | | Region |
| 163 | EVQLVQSGAEVKKPGESLKISCKGSGYSFTSYWIGWVRQ | WO2018/195506 |
| | MPGKGLEWMGIIYPGDADARYSPSFQGQVTISADKSISTA | 13E7 (SST202443) |
| | YLQWSSLKASDTAMYFCARRRQGIFGDALDF | Heavy Chain |
| | WGQGTLVTVSS | Variable Region |
| 164 | EIVMTQSPATLSVSPGERATLSCRASQSVSSNLAWFQQKP | WO2018/195506 |
| | GQAPRLLIYGASTRATGIPARFSVSGSGTEFTLTISSLQSED | 13E7 14C12 |
| | FAVYYCLQDNNWPPTFGPGTKVDIK | Light Chain Variable |
| | | Region |
| 165 | EVQLVQSGAEVKKPGESLMISCKGSGYSFTSYWIGWVR | WO2018/195506 |
| | QMPGKGLEWMGIIYPGDSDTRYSPSFQGQVTISADKSIST | 13E7 14C12 |
| | AYLQWSSLKASDTAMYFCARRRQGIWGDALDFWGQGT | Heavy Chain |
| | LVTVSS | Variable Region |
| 166 | RASQGISSWLA | WO2018/195506 |
| | | 6E7 |
| | | CDR-L1 |
| 167 | AASSLQN | WO2018/195506 |
| | | 6E7 |
| | | CDR-L2 |
| 168 | QQADSFPRT | WO2018/195506 |
| | | 6E7 |
| | | CDR-L3 |
| 169 | SYWIA | WO2018/195506 |
| | | 6E7 |
| | | CDR-H1 |
| 170 | IIYPGDSDTRYSPSFQG | WO2018/195506 |
| | | 6E7 |
| | | CDR-H2 |
| 171 | QRTFYYDSSDYFDY | WO2018/195506 |
| | | 6E7 |
| | | CDR-H3 |
| 172 | RASQGISNYLA | WO2018/195506 |
| | | 5E3 |
| | | CDR-L1 |
| 173 | AASSLQS | WO2018/195506 |
| | | 5E3 |
| | | CDR-L2 |
| 174 | QQYSTYPFT | WO2018/195506 |
| | | 5E3 |
| | | CDR-L3 |
| 175 | GYYIH | WO2018/195506 |
| | | 5E3 |
| | | CDR-H1 |
| 176 | WINPYSGGTTSAQKFQG | WO2018/195506 |
| | | 5E3 |
| | | CDR-H2 |
| 177 | DGGYLALYGTDV | WO2018/195506 |
| | | 5E3 |
| | | CDR-H3 |
| 178 | KSSQSVLYSSNNKHFLA | WO2018/195506 |
| | | 24G6 |
| | | CDR-L1 |
| 179 | WASTRES | WO2018/195506 |
| | | 24G6 |
| | | CDR-L2 |
| 180 | QQYYSTPLT | WO2018/195506 |
| | | 24G6 |
| | | CDR-L3 |
| 181 | SYAMS | WO2018/195506 |
| | | 24G6 |
| | | CDR-H1 |
| 182 | AISGSGGSTYYADSVKG | WO2018/195506 |
| | | 24G6 |
| | | CDR-H2 |
| 183 | AYTPMAFFDY | WO2018/195506 |
| | | 24G6 |
| | | CDR-H3 |
| 184 | RASQSVNNNLA | WO2018/195506 |
| | | 25F12 |
| | | CDR-L1 |
| 185 | GASTRAT | WO2018/195506 |
| | | 25F12 |
| | | CDR-L2 |
| 186 | QQYNNWPRT | WO2018/195506 |
| | | 25F12 |
| | | CDR-L3 |
| 187 | SYYWS | WO2018/195506 |
| | | 25F12 |
| | | CDR-H1 |
| 188 | EINHSGNTNYNPSLKS | WO2018/195506 |
| | | 25F12 |
| | | CDR-H2 |
| 189 | EGYYDILTGYHDAFDI | WO2018/195506 |
| | | 25F12 |
| | | CDR-H3 |

Any of the antibodies of the present disclosure may be produced by a cell line. In some embodiments, the cell line may be a mammalian cell line. In certain embodiments, the cell line may be a hybridoma cell line. In other embodiments, the cell line may be a yeast cell line. Any cell line known in the art suitable for antibody production may be used to produce an antibody of the present disclosure. Exemplary cell lines for antibody production are described throughout the present disclosure.

Antibody Fragments

Certain aspects of the present disclosure relate to antibody fragments that bind to one or more of human TREM2, a naturally occurring variant of human TREM2, and a disease variant of human TREM2. In some embodiments, the antibody fragment is an Fab, Fab′, Fab′-SH, F(ab′)2, Fv or scFv fragment.

Antibody Frameworks

Any of the antibodies described herein further include a framework. In some embodiments, the framework is a human immunoglobulin framework. For example, in some embodiments, an antibody (e.g., an anti-TREM2 antibody) comprises HVRs as in any of the above embodiments and further comprises an acceptor human framework, e.g., a human immunoglobulin framework or a human consensus framework. Human immunoglobulin frameworks may be part of the human antibody, or a non-human antibody may be humanized by replacing one or more endogenous frameworks with human framework region(s). Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Antibody Preparation

Anti-TREM2 antibodies of the present disclosure can encompass polyclonal antibodies, monoclonal antibodies, humanized and chimeric antibodies, human antibodies, antibody fragments (e.g., Fab, Fab′-SH, Fv, scFv, and F(ab′)₂), bispecific and polyspecific antibodies, multivalent antibodies, library derived antibodies, antibodies having modified effector functions, fusion proteins containing an antibody portion, and any other modified configuration of the immunoglobulin molecule that includes an antigen recognition site, such as an epitope having amino acid residues of a TREM2 protein of the present disclosure, including glycosylation variants of antibodies, amino acid sequence variants of antibodies, and covalently modified antibodies. The anti-TREM2 antibodies may be human, murine, rat, or of any other origin (including chimeric or humanized antibodies).

(1) Polyclonal Antibodies

Polyclonal antibodies, such as anti-TREM2 polyclonal antibodies, are generally raised in animals by multiple subcutaneous (sc) or intraperitoneal (ip) injections of the relevant antigen and an adjuvant. It may be useful to conjugate the relevant antigen (e.g., a purified or recombinant TREM2 protein of the present disclosure) to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the desired antigen, immunogenic conjugates, or derivatives by combining, e.g., 100 μg (for rabbits) or 5 μg (for mice) of the protein or conjugate with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant-cell culture as protein fusions. Also, aggregating agents such as alum are suitable to enhance the immune response.

(2) Monoclonal Antibodies

Monoclonal antibodies, such as anti-TREM2 monoclonal antibodies, are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the anti-TREM2 monoclonal antibodies may be made using the hybridoma method first described by Köhler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization (e.g., a purified or recombinant TREM2 protein of the present disclosure). Alternatively, lymphocytes may be immunized in vitro. Lymphocytes then are fused with an immortal cell line, such as myeloma cells, using a suitable fusing agent, such as polyethylene glycol, to form a hybridoma cell (Goding, Monoclonal Antibodies: Principles and Practice, pp. 59-103 (Academic Press, 1986)).

The culture medium in which the hybridoma cells are cultured can be assayed for the presence of monoclonal antibodies directed against the desired antigen (e.g., a TREM2 protein of the present disclosure), e.g., as determined by immunoprecipitation or by an in vitro binding assay, such as radioimmunoassay (RIA) or enzyme-linked assay (ELISA). Such techniques and assays are known in the in art.

After hybridoma cells are identified that produce antibodies of the desired specificity, affinity, and/or activity, the clones may be subcloned, and monoclonal antibodies secreted by the subclones may be separated from the culture medium, ascites fluid, or serum by conventional immunoglobulin purification procedures such as, for example, protein A-Sepharose chromatography, hydroxylapatite chromatography, gel electrophoresis, dialysis, affinity chromatography, and other methods as described above.

Anti-TREM2 monoclonal antibodies may also be made by recombinant DNA methods, e.g., as described above. DNA encoding the monoclonal antibodies is readily isolated and sequenced using conventional procedures (e.g., by using oligonucleotide probes that specifically bind to genes encoding the heavy and light chains of murine antibodies). The hybridoma cells serve as a preferred source of such DNA. Once isolated, the DNA may be placed into expression vectors, which are then transfected into host-cells such as E. coli cells, simian COS cells, Chinese hamster ovary (CHO) cells, or myeloma cells that do not otherwise produce immunoglobulin protein, in order to synthesize monoclonal antibodies in such recombinant host-cells. Review articles on recombinant expression in bacteria of DNA encoding the antibody include Skerra et al., Curr. Opin. Immunol., 5:256-262 (1993) and Phickthun, Immunol. Rev. 130:151-188 (1992).

In certain embodiments, anti-TREM2 antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) described the isolation of murine and human antibodies, respectively, from phage libraries. Subsequent publications describe the production of high affinity (nanomolar (“nM”) range) human antibodies by chain shuffling (Marks et al., Bio Technology, 10:779-783 (1992)), as well as combinatorial infection and in vivo recombination as a strategy for constructing very large phage libraries (Waterhouse et al., Nucl. Acids Res., 21:2265-2266 (1993)).

The DNA encoding antibodies or fragments thereof may also be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(3) Humanized Antibodies

Anti-TREM2 antibodies of the present disclosure or antibody fragments thereof may further include humanized or human antibodies. Humanized forms of non-human (e.g., murine) antibodies are chimeric immunoglobulins, immunoglobulin chains or fragments thereof (such as Fab, Fab′-SH, Fv, scFv, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally will also comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Certain methods for humanizing non-human anti-TREM2 antibodies are known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies may impact immunogenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Nat'l Acad. Sci. USA 89:4285 (1992); Presta et al., J. Immunol. 151:2623 (1993).

Humanized antibodies preferably retain high affinity for the antigen and other favorable biological properties. To achieve this goal, according to a preferred method, humanized antibodies are prepared by a process of analyzing the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen or antigens (e.g., TREM2 proteins of the present disclosure), is achieved. In general, the CDR residues are directly and most substantially involved in influencing antigen binding.

Various forms of the humanized anti-TREM2 antibody are contemplated. For example, the humanized anti-TREM2 antibody may be an antibody fragment, such as an Fab, or an intact antibody, such as an intact IgG1 antibody.

(4) Antibody Fragments

In certain embodiments, there are advantages to using anti-TREM2 antibody fragments, rather than whole anti-TREM2 antibodies. In some embodiments, smaller fragment sizes allow for rapid clearance and better brain penetration.

Various techniques have been developed for the production of antibody fragments. Traditionally, these fragments were derived via proteolytic digestion of intact antibodies (see, e.g., Morimoto et al., J. Biochem. Biophys. Method. 24:107-117 (1992); and Brennan et al., Science 229:81 (1985)). However, these fragments can now be produced directly by recombinant host-cells, for example, using nucleic acids encoding anti-TREM2 antibodies of the present disclosure. Fab, Fv and scFv antibody fragments can all be expressed in and secreted from E. coli, thus allowing the straightforward production of large amounts of these fragments. Anti-TREM2 antibody fragments can also be isolated from the antibody phage libraries as discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)2 fragments can be isolated directly from recombinant host-cell culture. Production of Fab and F(ab′)₂ antibody fragments with increased in vivo half-lives are described in U.S. Pat. No. 5,869,046. In other embodiments, the antibody of choice is a single chain Fv fragment (scFv). See WO 93/16185; U.S. Pat. Nos. 5,571,894 and 5,587,458. The anti-TREM2 antibody fragment may also be a “linear antibody,” e.g., as described in U.S. Pat. No. 5,641,870. Such linear antibody fragments may be monospecific or bispecific.

(5) Bispecific and Polyspecific Antibodies

Bispecific antibodies (BsAbs) are antibodies that have binding specificities for at least two different epitopes, including those on the same or another protein (e.g., one or more TREM2 proteins of the present disclosure). Alternatively, one part of a BsAb can be armed to bind to the target TREM2 antigen, and another can be combined with an arm that binds to a second protein. Such antibodies can be derived from full-length antibodies or antibody fragments (e.g., F(ab′)₂ bispecific antibodies).

(6) Effector Function Engineering

It may also be desirable to modify an anti-TREM2 antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH 2 domain of IgG) of the antibody. In some embodiments, the effector function is impaired by modifying regions such as 233-236, 297, and/or 327-331 of human IgG as described in PCT WO 99/58572 and Armour et al., Molecular Immunology 40: 585-593 (2003); Reddy et al., J. Immunology 164:1925-1933 (2000). In other embodiments, it may also be desirable to modify an anti-TREM2 antibody of the present disclosure to modify effector function to increase finding selectivity toward the ITIM-containing FcgRIIb (CD32b) to increase clustering of TREM2 antibodies on adjacent cells without activating humoral responses including antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

(7) Other Amino Acid Sequence Modifications

Amino acid sequence modifications of anti-TREM2 antibodies of the present disclosure, or antibody fragments thereof, are also contemplated. For example, it may be desirable to improve the binding affinity and/or other biological properties of the antibodies or antibody fragments. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics (i.e., the ability to bind or physically interact with a TREM2 protein of the present disclosure). The amino acid changes also may alter post-translational processes of the antibody, such as changing the number or position of glycosylation sites.

A useful method for identification of certain residues or regions of the anti-TREM2 antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the target antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity.

Amino acid sequence insertions include amino- (“N”) and/or carboxy- (“C”) terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table C below under the heading of “preferred substitutions”. If such substitutions result in a change in biological activity, then more substantial changes, denominated “exemplary substitutions” in Table C, or as further described below in reference to amino acid classes, may be introduced and the products screened.

TABLE C
Amino acid substitutions

| Original Residue | Exemplary Substitutions | Preferred Substitutions |
|---|---|---|
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | Ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

A particularly preferred type of substitutional variant involves substituting one or more hypervariable region residues of a parent antibody (e.g. a humanized or human anti-TREM2 antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen (e.g., a TREM2 protein of the present disclosure). Such contact residues and neighboring residues are candidates for substitution according to the techniques elaborated herein. Once such variants are generated, the panel of variants is subjected to screening as described herein and antibodies with superior properties in one or more relevant assays may be selected for further development. Affinity maturation may also be performed by employing a yeast presentation technology such as that disclosed in, for example, WO2009/036379A2; WO2010105256; WO2012009568; and Xu et al., Protein Eng. Des. Sel., 26(10): 663-70 (2013).

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

(8) Other Antibody Modifications

Anti-TREM2 antibodies of the present disclosure, or antibody fragments thereof, can be further modified to contain additional non-proteinaceous moieties that are known in the art and readily available, or to contain different types of drug conjugates that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

Drug conjugation involves coupling of a biological active cytotoxic (anticancer) payload or drug to an antibody that specifically targets a certain tumor marker (e.g. a protein that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue, OncLive Jul. 5, 2012: ADC Review on antibody-drug conjugates; and Ducry et al., (2010). Bioconjugate Chemistry 21 (1): 5-13).

(9) Binding Assays and Other Assays

Anti-TREM2 antibodies of the present disclosure may be tested for antigen binding activity, e.g., by known methods such as ELISA, Western blot, etc.

Detailed exemplary methods for mapping an epitope to which an antibody binds are provided in Morris (1996) “Epitope Mapping Protocols,” in Methods in Molecular Biology vol. 66 (Humana Press, Totowa, N.J.).

### Nucleic Acids, Vectors, and Host Cells

Anti-TREM2 antibodies of the present disclosure may be produced using recombinant methods and compositions, e.g., as described in U.S. Pat. No. 4,816,567. In some embodiments, isolated nucleic acids having a nucleotide sequence encoding any of the anti-TREM2 antibodies of the present disclosure are provided. Such nucleic acids may encode an amino acid sequence containing the VL and/or an amino acid sequence containing the VH of the anti-TREM2 antibody (e.g., the light and/or heavy chains of the antibody). In some embodiments, one or more vectors (e.g., expression vectors) containing such nucleic acids are provided. In some embodiments, a host cell containing such nucleic acid is also provided. In some embodiments, the host cell contains (e.g., has been transduced with): (1) a vector containing a nucleic acid that encodes an amino acid sequence containing the VL of the antibody and an amino acid sequence containing the VH of the antibody, or (2) a first vector containing a nucleic acid that encodes an amino acid sequence containing the VL of the antibody and a second vector containing a nucleic acid that encodes an amino acid sequence containing the VH of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-TREM2 antibody of the present disclosure are provided. In some embodiments, the method includes culturing a host cell of the present disclosure containing a nucleic acid encoding the anti-TREM2 antibody, under conditions suitable for expression of the antibody. In some embodiments, the antibody is subsequently recovered from the host cell (or host cell culture medium).

For recombinant production of an anti-TREM2 antibody of the present disclosure, a nucleic acid encoding the anti-TREM2 antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the anti-TREM2 antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-TREM2 antibody of the present disclosure.

Suitable host cells for cloning or expression of antibody-encoding vectors include prokaryotic or eukaryotic cells. For example, anti-TREM2 antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B.K.C. Lo, ed., Humana Press, Totowa, N.J., 2003), pp. 245-254, describing expression of antibody fragments in E. coli.). After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross, Nat. Biotech. 22:1409-1414 (2004); and Li et al., Nat. Biotech. 24:210-215 (2006)).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CVI); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

### Pharmaceutical Compositions and Formulations

Provided herein are pharmaceutical compositions comprising an anti-TREM2 antibody of the present disclosure and a pharmaceutically acceptable carrier. In some embodiments, provided herein are pharmaceutical compositions comprising the anti-TREM2 antibody of the present disclosure having the desired degree of purity in a physiologically acceptable carrier, excipient or stabilizer (Remington's Pharmaceutical Sciences (1990) Mack Publishing Co., Easton, Pa.). Acceptable carriers, excipients, or stabilizers are nontoxic to recipients at the dosages and concentrations employed.

In various embodiments, pharmaceutical compositions comprising an anti-TREM2 antibody are provided in formulations with a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

### Pharmaceutical Dosages and Administration

An anti-TREM2 antibody provided herein can be administered by any suitable means, including parenteral, intrapulmonary, intranasal, intralesional administration, intracerobrospinal, intracranial, intraspinal, intrasynovial, intrathecal, oral, topical, or inhalation routes. Parenteral infusions include intramuscular, intravenous administration as a bolus or by continuous infusion over a period of time, intraarterial, intra-articular, intraperitoneal, or subcutaneous administration. In some embodiments, the administration is intravenous administration. In some embodiments, the administration is subcutaneous. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

For the prevention or treatment of disease, the appropriate dosage of an anti-TREM2 antibody will depend on the type of disease to be treated, the particular antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

### Kits/Articles of Manufacture

The present disclosure also provides kits containing an isolated antibody of the present disclosure (e.g., an anti-TREM2 antibody described herein), or a functional fragment thereof. Kits of the present disclosure may include one or more containers comprising a purified antibody of the present disclosure. In some embodiments, the kits further include instructions for use in accordance with the methods of this disclosure. In some embodiments, these instructions comprise a description of administration of the isolated antibody of the present disclosure (e.g., an anti-TREM2 antibody described herein) to prevent, reduce risk, or treat an individual having a demyelination disease, disorder, or injury. In some embodiments, the demyelination disease, disorder, or injury is multiple sclerosis, optic neuritis, neuromyelitis optica (Devic's disease), transverse myelitis, acute disseminated encephalomyelitis, adrenoleukodystrophy, or adrenomyeloneuropathy. In some embodiments, the demyelination disease, disorder, or injury is multiple sclerosis.

In some embodiments, the instructions comprise a description of how to detect TREM2, for example in an individual, in a tissue sample, or in a cell. The kit may further comprise a description of selecting an individual suitable for treatment based on identifying whether that individual has the disease and the stage of the disease.

In some embodiments, the kits may further include another antibody of the present disclosure (e.g., at least one antibody that specifically binds to an inhibitory checkpoint molecule, at least one antibody that specifically binds to an inhibitory cytokine, and/or at least one agonistic antibody that specifically binds to a stimulatory checkpoint protein) and/or at least one stimulatory cytokine. In some embodiments, the kits may further include instructions for using the antibody and/or stimulatory cytokine in combination with an isolated antibody of the present disclosure (e.g., an anti-TREM2 antibody described herein), instructions for using the isolated antibody of the present disclosure in combination with an antibody and/or stimulatory cytokine, or instructions for using an isolated antibody of the present disclosure and an antibody and/or stimulatory cytokine, according to any methods of this disclosure.

The instructions generally include information as to dosage, dosing schedule, and route of administration for the intended treatment. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a disease of the present disclosure. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example, the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). At least one active agent in the composition is an isolated antibody of the present disclosure (e.g., an anti-TREM2antibody described herein). The container may further comprise a second pharmaceutically active agent.

Kits may optionally provide additional components such as buffers and interpretive information. Normally, the kit comprises a container and a label or package insert(s) on or associated with the container.

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Trem2 Haploinsufficiency Results in Inefficient Myelin Debris Clearance

Homozygous Trem2^{-/-} mice exhibit a significant defect in clearance of myelin debris in a cuprizone (CPZ)-induced model of demyelination compared to Trem2‘i’ mice (see, e.g., Cantoni, Bollman et al. 2015, Poliani, Wang et al. 2015). Whether heterozygous (Trem2^{+/-}) mice would present the same or a milder phenotype than Trem2^{-/-} mice was evaluated in this Example.

### Materials and Methods

### Mice

Trem2+/+, Trem2^{+/-} and Trem2−/− mice (backcrossed 12 generations to the C57BL/6 background) were used. The three strains were bred in parallel. Mice were maintained under controlled conditions (19-22° C. and in a 12-h light/dark cycle with unrestricted access to food and water).

### CPZ-Induced Model of Demyelination

Trem2^{+/+}, Trem2^{+/-}, and Trem2^{-/-} mice were fed a 0.2% Cuprizone Diet (5C5N: Modified PicoLab® Rodent w/0.2% Cuprizone, TestDiet; cuprizone from Sigma (cod C9012-25G)) for 4 weeks or for 4 weeks followed by 3 days or 7 days or 14 days on regular chow (PicoLab Rodent Diet 20 #5053, Purina).

### Mouse Tissue Processing and Histological Analyses

Mice were anesthetized with ketamine (100 mgkg⁻¹) and perfused with 4% paraformaldehyde (PFA). Mouse brains were removed and post-fixed in 4% PFA for 24 hours, followed by immersion in 30% sucrose for 24-48 hours. The samples were then embedded in Optimal Cutting Temperature (OCT). Thick sections (5 m) were placed on glass slides and stained with solochrome cyanine to confirm the presence of a lesion as previously described (Kiernan, 1984). Sections were stained with the following primary antibodies: rabbit (Rb) anti-dMBP (Millipore, ab5864, 1:2000), Rb anti-Iba1 (Wako, 019-19741, 1:600), goat (Gt) anti-Iba1 (Novus, NB100-1028, 1:250), Rb anti-PDGFRa (ThermoFisher, PA5-16742, 1:50), Rb anti-OLIG2 (Milipore, AB9610, 1:300), mouse (Ms) anti-CNPase (Abcam, ab6319, 1:100), and an anti-SMI-31 antibody. AlexaFluor-conjugated secondary antibodies (Thermo Fisher, 1:400) were used. Some of the images were acquired with a Nikon Eclipse 90i fluorescent and bright field microscope equipped with 10×, 20×, and 60×objectives and analyzed with Metamorph 7.7 software. CNPase, SMI-31, and dMBP were analyzed as the percentage area of positive staining (number of positive pixels/mm²) within the region of interest. Iba1, PDGFRa, and OLIG2 were quantified as the density of cells in the region of interest (number of cells/mm²). For confocal analysis, images were acquired with an Olympus FV1200 laser scanning confocal microscope (Olympus-America Inc., Waltham, Mass.) equipped with a PlanApoN 60X, 1.4 NA super corrected oil objective. The Olympus FV1200 confocal microscope was equipped with five detectors: two spectral and one filter-based and two gallium arsenide phosphide (GaAsP) photo-multiplier tubes (PMTs). The 405-, 488-, and 559-nm diode lasers and 635-nm HeNe (helium neon) lasers were used with an optimal pinhole of 1 airy unit to acquire images. Images were finally processed with ImageJ software.

### Immunohistochemistry Analysis of Myelin Debris Accumulation

To assess the amount of myelin debris accumulation in the corpus callosum (CC), a site of particularly profound and consistent demyelination in the CPZ model (Matsushima and Morell 2001), degraded myelin basic protein (dMBP) was analyzed by immunohistochemistry using an antibody able to bind MBP epitopes exposed only after myelin degradation (Matsuo, Lee et al. 1997).

### Statistical Analysis

Prism 8.0 (GraphPad Software) was used for statistical analyses. Data shown are the mean±standard deviation (s.d.), with P<0.05 considered statistically significant. Group differences were analyzed by one-way analysis of variance (ANOVA) followed by Sidak's or Benjamini, Krieger and Yekutieli correction multiple comparisons tests, or by two-way ANOVA followed by Tukey's multiple comparisons test for multiple groups.

### Results

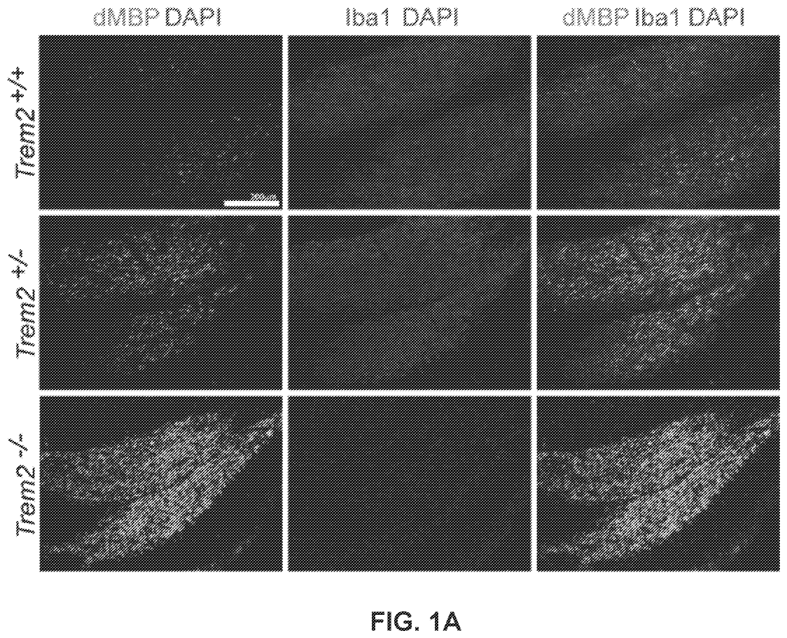

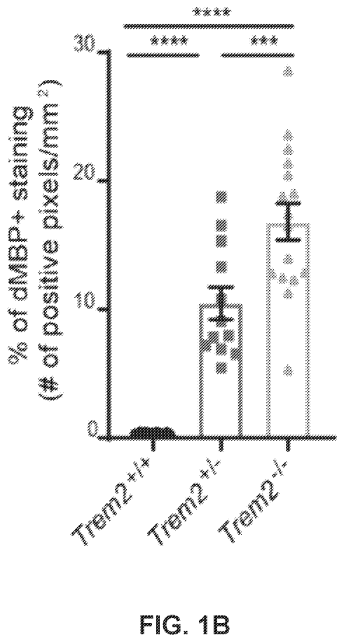

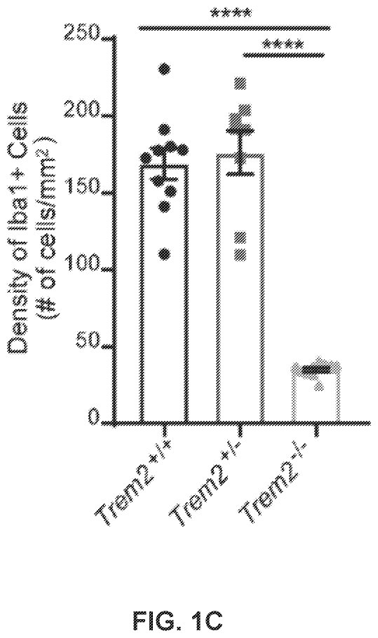

As shown in FIGS. 1A-1B, Trem2^{-/-} mice after 4 weeks on CPZ displayed a significant increase in dMBP signal when compared to Trem2^{+/+} mice. The increased dMBP signal in Trem2^{-/-} mice was accompanied by a significant reduction in Iba1+ microglia (see FIGS. 1A and 1C). The numbers of OPCs in Trem2−/− mice were also significantly reduced compared to Trem2^{+/+} mice (data not shown). No differences were found in astrocyte activation, quantified as glial fibrillary acidic protein (GFAP) positive cells, between Trem2^{+/+} and Trem2−/− mice (data not shown).

Notably, Trem2^{+/-} mice displayed a phenotype which was intermediate between Trem2^{+/+} and Trem2−/− mice. For example, as shown in FIGS. 1A-1B, Trem2^{+/-} mice exhibited a significant increase in dMBP signal when compared to Trem2^{+/+} mice, but the increase was significantly smaller than that observed in Trem2−/− mice. Trem2+/− mice also showed less PDGFRα⁺ OPCs compared to Trem2‘i’ mice (data not shown). No differences were noted in the number of Iba1+ cells in the corpus callosum of Trem2^{+/-} mice compared to Trem2″ mice (FIGS. 1A and 1C), in contrast to the significant reduction of the microglia number observed in Trem2−/− mice compared to Trem2^{+/+} mice. The number of GFAP⁺ cells in the corpus callosum was not different between Trem2^{+/- and} Trem2^{+/+} mice (data not shown).

The results described in this Example showed that Trem2 haploinsufficiency (i.e., loss of one copy of TREM2) resulted in an intermediate phenotype, suggesting that myelin removal by microglia is sensitive to TREM2 copy number. In particular, Trem2 haploinsufficiency led to inefficent myelin debris clearance and reduced OPC recruitment, even if it did not impact the number of microglia accumulating in the corpus callosum. These results suggested that one copy of TREM2 is sufficient to sustain microglia expansion after CPZ-induced damage, but cannot support a fully functional response by microglia to clear myelin debris derived from massive oligodendrocyte death.

Overall, the results described in this Example showed that that the presence of a fully functional TREM2 receptor on microglia is optimal for the efficient myelin debris removal and OPC recruitment. Moreover, the discovery that Trem2 haploinsufficient mice (Trem2^{+/-} mice) displayed an intermediate phenotype, having defects in myelin debris clearance, provided a model for investigating the effect of a TREM2 agonist antibody on this process. TREM2 protein is present in Trem2 haploinsufficient mice, albeit at presumably decreased levels compared to Trem2^{+/+} mice, and therefore a TREM2 agonist antibody can be tested.

### Example 2: Characterization of the Effects of an Agonist Anti-TREM2 Antibody on TREM2 Signaling

In this Example, the effects of an agonist anti-TREM2 antibody on TREM2 signaling in vitro and in vivo were analyzed.

### Materials and Methods

### Generation of Anti-TREM2 Antibodies

Monoclonal antibodies targeting mouse TREM2 were generated by immunizing mice genetically deficient in TREM2 (Trem2−/−) with recombinant TREM2 protein, followed by hybridoma generation. Anti-TREM2 antibody 7E5, which has a mouse IgG1 Fc region (mIgG1), was selected for its specific binding to wild type (Trem2+/+) but not Trem2^{-/-} bone marrow-derived macrophages (BMDMs).

Anti-TREM2 antibody 7E5 has a light chain variable domain with the following sequence:

(SEQ ID NO: 50)
DVZMTQTPLTLSVTIGQPASISCKSSQSLLYSNGKTFLSWLLQRPGQSP
KRLIYLVSKLDSGVPDRFAGSGSGTDFTLKISRLEADDLGIYYCMQGTH
FPLTFGAGTKLELK.

Anti-TREM2 antibody 7E5 has a heavy chain variable domain with the following sequence:

(SEQ ID NO: 51)
EVKLEESGGGLVQPGGSMKLSCAASGFTFSDAWMGWVRQSPEKGLEWVA
EIRDKVKNHATYYAESVKGRFTISRDDSKSTVYLQMNTLRADDTGIYYC
RLGVFDYWGQGTTLTVSS.

In the above sequences of the light chain and heavy chain variable domains of anti-TREM2 antibody 7E5, the HVR sequences have been underlined.

### Bone Marrow-Derived Macrophages

Bone marrow-derived macrophages (BMDMs) were obtained by flushing tibial and femoral marrow cells with cold PBS 2% FBS. Red blood cells were lysed using ACK lysing buffer (Thermo Fisher), and after two washes in PBS 2% FBS, the cells were re-suspended in complete media (RPMI, 10% FBS, Pen/Strep, L-glutamine, non-essential amino acid) with (50 ng/ml) murine M-CSF (m-MCSF) to obtain differentiated macrophages after 6 days. Adherent macrophages were detached with 1 mM EDTA in PBS.

### In Vitro and In Vivo Immunoprecipitation and Immunoblotting

For in vitro immunoprecipitation experiments, BMDM cells were starved for 4 h in RPMI with 1% FBS. 10×10⁶ cells were incubated for 15 min at 4° C. with 1 μg of control antibody or anti-TREM2 antibody 7E5. Cells were then washed and incubated at 37° C. in the presence of goat-anti mouse IgG (1.5 μg for 1×10⁶ cells). After stimulation, cells were lysed with lysis buffer (1% n-Dodecyl-β-D-Maltoside, 50 Mm Tris-HCl (pH 8.0), 150 mM NaCl, 1 mM EDTA, 1.5 mM MgCl2, 10% glycerol, protease and phosphatase inhibitors) and immunoprecipitated with an anti-TREM2 antibody that binds a different domain of TREM2 than antibody 7E5 (rat anti-h/m-TREM2, clone 237920, R&D System) or isotype control.

For in vivo immunoprecipitation experiments, 6-8 week old C57BL/6 mice were injected intraperitoneally with 3 ml of 3% thioglycolate. After 3 days, when the peritoneal cavity was enriched with CD11b⁺F4/80⁺ macrophages expressing TREM2, mice were injected with control antibody or anti-TREM2 antibody 7E5 (40 mg/kg). 24 hours after antibody injection, peritoneal macrophages were collected, immediately lysed in lysis buffer (as described above), and immunoprecipitated with rat anti-h/m TREM2 antibody (R&D System, clone 237920).

For both in vitro and in vivo experiments, precipitated proteins were fractionated by SDS-PAGE in non-reducing conditions, transferred to PVDF membranes, and probed with an anti-phosphotyrosine antibody (4G10, Millipore). TREM2 is not detected in non-reducing conditions. To confirm that all substrates were adequately immunoprecipitated, whole cell lysates from each sample were also fractionated by SDS-PAGE in reducing conditions and immunoblotted with an anti-actin antibody (actin, sc-47778 Santa Cruz).

### NFAT-Luciferase Reporter Assay

A stable BW5147.G.1.4 (ATCC® TIB48™) (BWZ) cell line expressing both mouse TREM2 and DAP12 was infected with a Cignal PLenti NFAT-Luciferase virus (Qiagen) to generate a stable mouse TREM2 reporter cell line that induces luciferase signaling upon TREM2 activation. The activity of the reporter was validated using phorbol 12-myristate 13-acetate (PMA) (0.05 μg/ml) and ionomycin (0.25 μM). To test whether anti-TREM2 antibody 7E5 induced signaling, 5 μg/ml of soluble anti-TREM2 or control antibodies were added to each well of 96-well culture plates together with 100,000 cells/well and incubated for 4-6 hours at 37° C. in Dulbecco's Modified Eagle Medium (DMEM). Luciferase activity was measured by removing media and adding 50 μl of PBS and 50 μl of OneGlo Reagent (Promega). The mixtures were then incubated for 3 minutes at room temperature on a plate shaker to lyse the cells. Luciferase signal was measured using a BioTek plate reader. Data were analyzed using GraphPad Prism.

To test myelin-induced signaling, human myelin was diluted in PBS to 200 μg/ml, titrated onto a 96-well tissue culture plate and incubated overnight at 4° C. The next morning, the solution was removed, and plates were washed three times with 200 μl PBS. Plates were air dried, and BWZ cells (with or without antibodies) were added, incubated, and analyzed as described above.

### Myelin Production

Human myelin was prepared and stored in lyophilized form at −80° C. Prior to use, myelin was suspended in DMEM to a final concentration of 2 mg/ml and dissolved by vortexing and sonication. Myelin was then irradiated with 10000 RADS to achieve sterility. Aliquots were stored at −80° C. for further use.

### Results

### In Vitro TREM2 Signaling

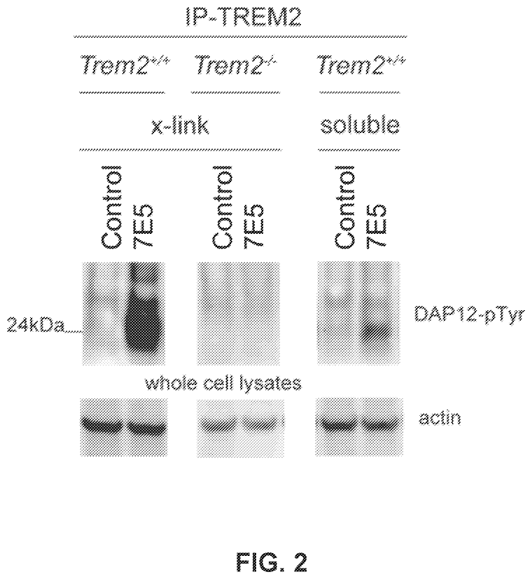

To test whether cross-linked anti-TREM2 antibody 7E5 induced signaling after receptor engagement, Trem2^{+/+} and Trem2^{-/-} BMDMs were stimulated in vitro with anti-TREM2 antibody 7E5 or isotype control antibody, followed by crosslinking with a secondary antibody, in order to induce receptor clustering. TREM2 was then immunoprecipitated, and DAP12 phosphorylation was analyzed. As shown in FIG. 2, cross-linked (“x-link”) anti-TREM2 antibody 7E5 induced phosphorylation of TREM2-associated DAP12 in Trem2^{+/+}, but not in Trem2−/− BMDMs. Treatment with isotype control antibody did not result in any DAP12 phosphorylation. These results demonstrated that cross-linked anti-TREM2 antibody 7E5 was a strong inducer of DAP12 phosphorylation and acted as an agonist mediating TREM2 signaling.

Whether soluble anti-TREM2 antibody 7E5 induced signaling after receptor engagement in the absence of cross-linking was also tested. As shown in FIG. 2, soluble anti-TREM2 antibody 7E5 (without secondary cross-linking) induced DAP12 phosphorylation. These results demonstrated that soluble (non-cross-linked) anti-TREM2 antibody 7E5 induced DAP12 phosphorylation and acted as an agonist mediating TREM2 signaling.

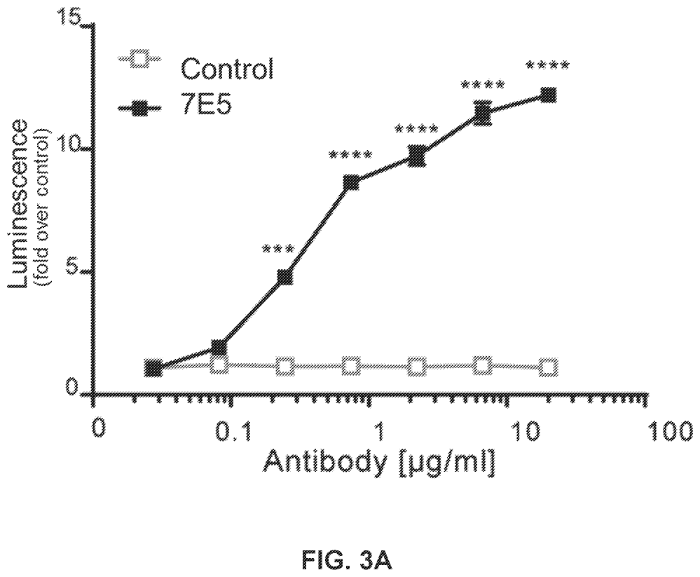

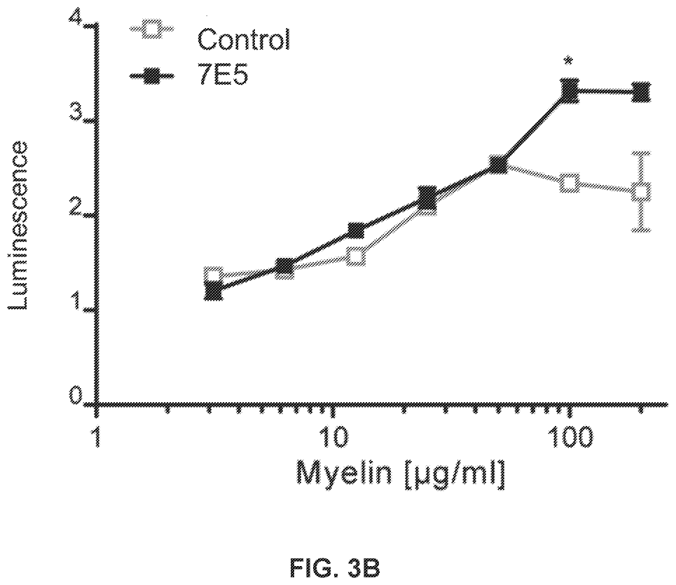

The ability of soluble anti-TREM2 antibody 7E5 to activate TREM2 signaling was also tested in a TREM2 reporter cell assay using BWZ cells expressing the mouse TREM2/DAP12 complex and transduced with a luciferase reporter gene under NFAT promoter control. As shown in FIG. 3A, anti-TREM2 antibody 7E5 induced TREM2 signaling in a dose-dependent manner, as demonstrated by incremental luciferase induction relative to isotype control antibody.

Subsequently, whether anti-TREM2 antibody 7E5 could block or enhance myelin-induced TREM2 signaling was tested using the TREM2 reporter cell assay described above. Anti-TREM2 antibody 7E5 or isotype control antibody were added to BWZ cells seeded on increasing concentrations of plate-bound myelin. As shown in FIG. 3B, at lower myelin concentrations (<100 μg/ml), anti-TREM2 antibody 7E5 did not modulate signaling differently than isotype control antibody. In contrast, at higher concentrations (>100 μg/ml), anti-TREM2 antibody 7E5 significantly increased myelin induced luciferase activation relative to isotype control antibody. These results showed that anti-TREM2 antibody 7E5 potentiated myelin-mediated TREM2 signaling activation in vitro.

### In Vivo TREM2 Signaling

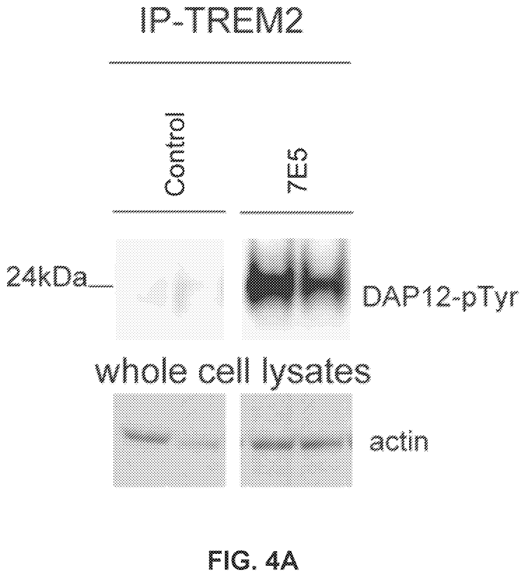

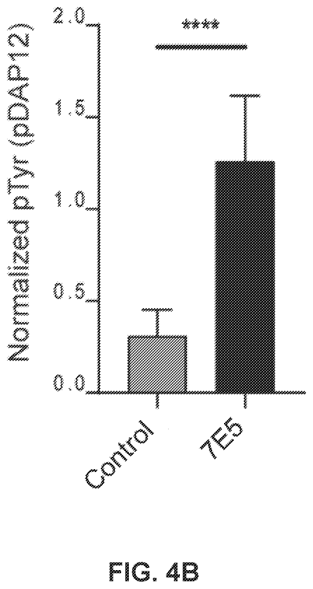

Next, whether anti-TREM2 antibody 7E5 was able to induce TREM2 signaling activation in vivo was investigated. Mice were injected with the anti-TREM2 antibody after thioglycollate-induced recruitment of macrophages to the peritoneal cavity. TREM2 was then immunoprecipitated, and DAP12 phosphorylation was analyzed. As shown in FIGS. 4A-4B, anti-TREM2 antibody 7E5 significantly induced phosphorylation of TREM2-associated DAP12 compared to the isotype control. These results demonstrated that anti-TREM2 antibody 7E5 is a potent inducer of TREM2 intracellular signaling in vivo (by soluble stimulation).

In conclusion, the results described in this Example demonstrated that anti-TREM2 antibody 7E5 was a potent TREM2 agonist both in vitro and in vivo, and that its agonistic activity was boosted by the presence of myelin.

### Example 3: Agonist Anti-TREM2 Antibody 7E5 Accelerates Myelin Debris Clearance

In this Example, the ability of anti-TREM2 antibody 7E5 to promote clearance of myelin debris caused by CPZ-induced demyelination in Trem2^{+/-} mice was assessed.

### Materials and Methods

### Administration of Anti-TREM2 Antibody 7E5

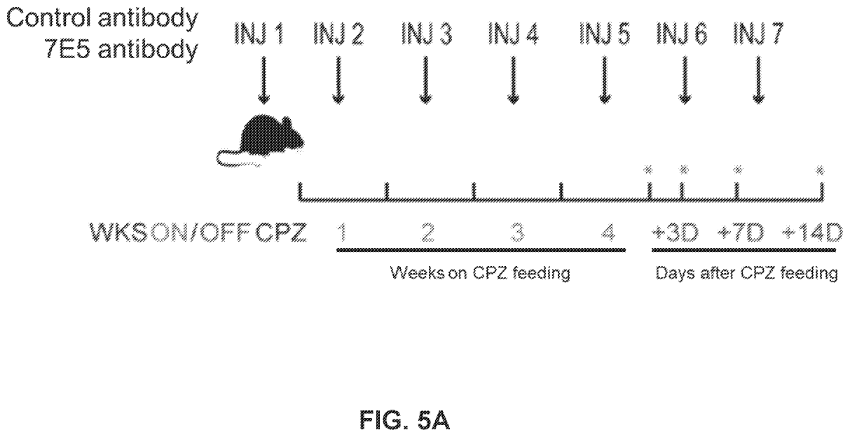

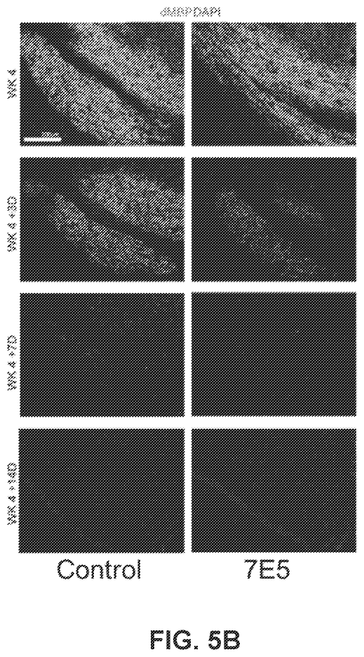

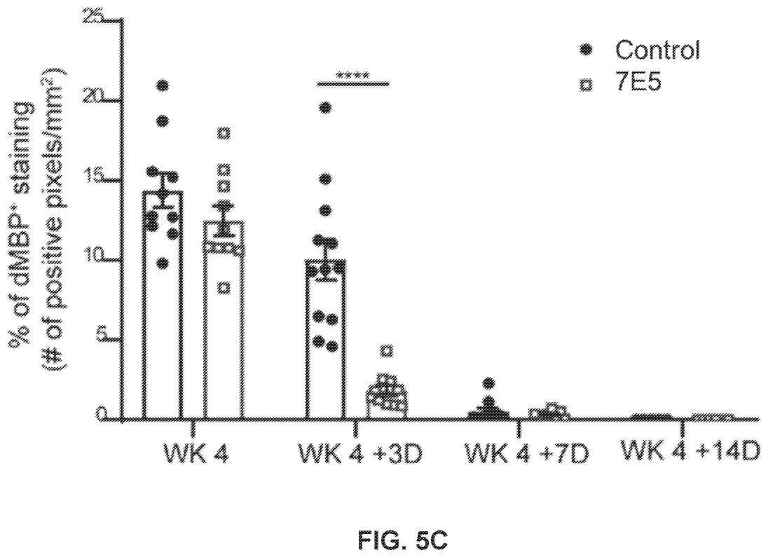

Trem2^{+/-} mice were treated with anti-TREM2 antibody 7E5 or an isotype control antibody given intraperitoneally at a dose of 80 mg/kg 4 days before starting CPZ feeding and then weekly throughout the experiment (see FIG. 5A). It was determined that the anti-TREM2 antibody reached the brain and was detectable in the corpus callosum and in the cortex (data not shown). Analyses were performed at 4 weeks after CPZ feeding (4WK; when the peak of demyelination was observed), and then at 3 days (WK 4+3D), 7 days (WK 4+7D), and 14 days (WK 4+14D) after CPZ withdrawal, when mice were fed regular chow to allow remyelination (see FIG. 5A).

### Results

Treatment in vivo with anti-TREM2 antibody 7E5 resulted in the amelioration of CPZ-induced pathology in Trem2+/− mice. Specifically, as shown in FIGS. 5B-5C, anti-TREM2 antibody 7E5 treatment enhanced myelin debris clearance relative to isotype control antibody, as demonstrated by a trend toward a reduction in dMBP staining at 4 weeks on CPZ. The enhanced myelin debris clearance observed in Trem2+/− mice treated with anti-TREM2 antibody 7E5 compared to Trem2^{+/-} mice treated with the control antibody was highly statistically significant at 3 days after CPZ withdrawal (FIG. 5C).

The results of the in vivo experiments described in this Example demonstrated that anti-TREM2 antibody 7E5 accelerated myelin clearance, suggesting that anti-TREM2 antibody 7E5 potentiated TREM2 function on microglia. Without wishing to be bound by theory, it is believed that the effect of anti-TREM2 antibody 7E5 on myelin clearance in Trem2^{+/-} mice was due to a direct effect of anti-TREM2 antibody 7E5 on microglia, the CNS cell type consistently shown to express TREM2 (Schmid, Sautkulis et al. 2002) and that is heavily involved in the response to damage after CPZ-induced demyelination.

### Example 4: Agonist Anti-TREM2 Antibody 7E5 Promoted Recruitment of Oligodendrocyte Precursor Cells and Differentiation into Mature Oligodendrocytes

Oligodendrocyte precursor cells (OPCs) sustain remyelination and counteract the oligodendrocyte loss that occurs during demyelination. In this Example, the effects of anti-TREM2 antibody 7E5 on OPC numbers and OPC differentiation into mature oligodendrocytes (OLs) in Trem2^{+/-} mice with CPZ-induced CNS demyelination were analyzed.

### Materials and Methods

CPZ-induced demyelination and administration of anti-TREM2 antibody 7E5 or an isotype control antibody to Trem2^{+/-} mice were carried out as described in Example 3.

Immunohistochemistry experiments were performed as described above.

### Results

### OPC Recruitment

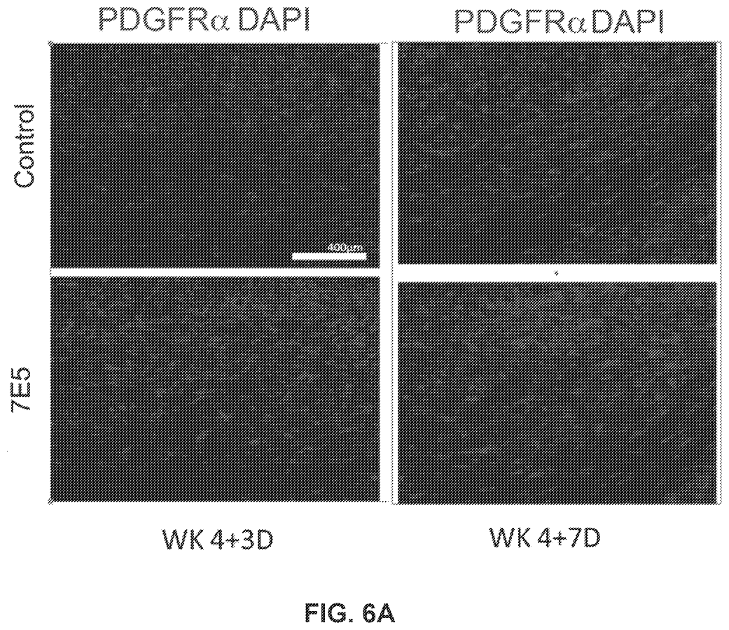

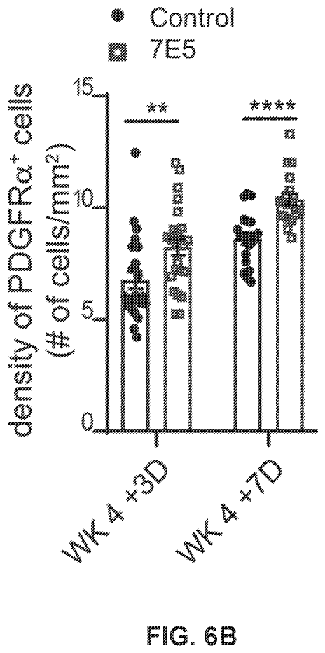

As shown in FIGS. 6A-6B, a statistically significant increase in PDGFRα*OPC density was observed in the corpus callosum of Trem2+/− mice after anti-TREM2 antibody 7E5 treatment at WK4+3D and WK4+7D compared to Trem2^{+/-} mice treated with control antibody. No differences in PDGFRα⁺ OPC proliferation, quantified as density of PDGFRa⁺BrdU⁺ cells, were observed at WK4+3D (data not shown).

OPC Differentiation into Mature OLs

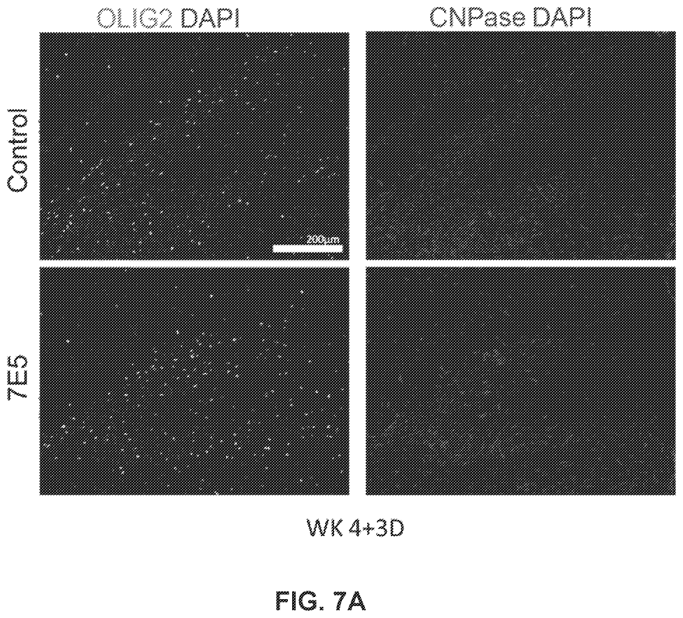

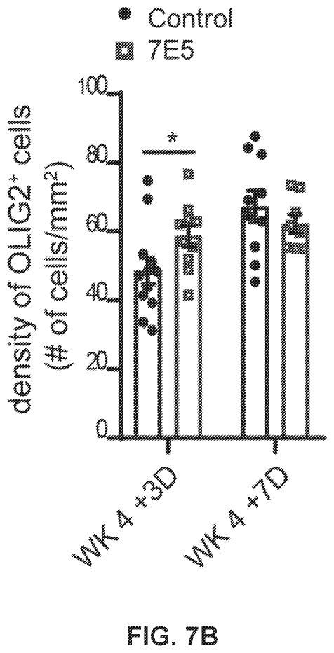

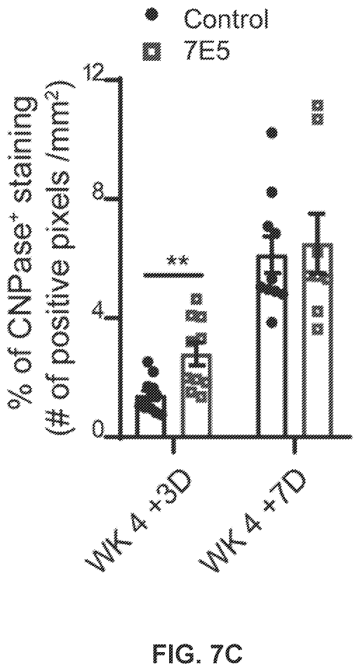

Expression of the OLIG2 and CNPase markers, which are specifically expressed by mature OLs, was analyzed to evaluate the effect of anti-TREM2 antibody 7E5 on oligodendrocyte maturation. As shown in FIGS. 7A-7C, statistically significant increases of both OLIG2⁺ cells (FIGS. 7A and 7B) and CNPase⁺ cells (FIGS. 7A and 7C) were observed at WK 4+3D after CPZ withdrawal in Trem2+/− mice administered anti-TREM2 antibody 7E5 compared to Trem2^{+/-} mice treated with control antibody.

The results described in this Example showed that anti-TREM2 antibody 7E5 administration to Trem2+/− mice with CPZ-induced demyelination resulted in the recruitment of oligodendrocyte precursor cells to the corpus callosum. In addition, anti-TREM2 antibody 7E5 promoted differentiation of OPCs into mature oligodendrocytes.

These results suggested that anti-TREM2 antibody 7E5 promoted the recruitment of OPCs from surrounding brain regions to the corpus callosum of Trem2^{+/-} mice with CPZ-induced CNS demyelination, rather than enhancing the proliferation of resident OPCs. However, even if an effect of anti-TREM2 antibody 7E5 on microglia proliferation was not observed at 4 weeks on CPZ, microglia proliferation could be affected by anti-TREM2 antibody 7E5 at an earlier time point, when it is more robust (see, e.g., (Gudi, Gingele et al. 2014).

### Example 5: Agonist Anti-TREM2 Antibody 7E5 Promotes Axonal Health in Mice with CPZ-Induced CNS Demyelination

In this Example, the effects of anti-TREM2 antibody 7E5 on axonal health in the corpus callosum of Trem2^{+/-} mice with CPZ-induced CNS demyelination were analyzed.

### Materials and Methods

CPZ-induced demyelination and administration of anti-TREM2 antibody 7E5 or an isotype control antibody to Trem2^{+/-} mice were carried out as described in Example 3.

Immunohistochemistry experiments were performed as described above.

### Results

### SAM-31 Expression

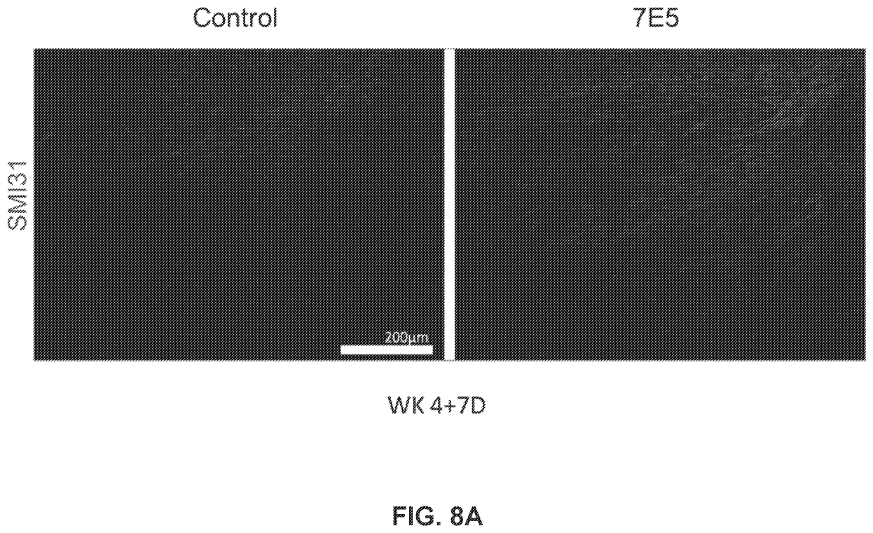

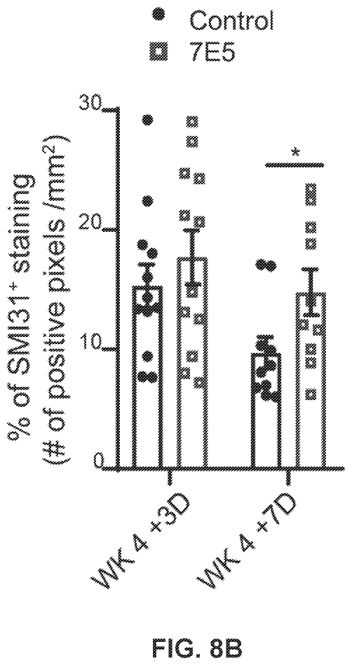

As shown in FIGS. 8A-8B, a statistically significant increase in SMI-31 expression, which is a marker for phosphorylated neurofilaments, was observed at WK 4+7D after CPZ withdrawal in Trem2+/− mice administered anti-TREM2 antibody 7E5 compared to Trem2^{+/-} mice treated with control antibody.

The presence of phosphorylated neurofilaments is an indicator of axonal health. Therefore, these results showed that anti-TREM2 antibody 7E5 promoted axonal health in the corpus callosum of Trem2^{+/-} mice with CPZ-induced CNS demyelination.

What is claimed is:

1. A method for treating or preventing a central nervous system (CNS) demyelination disease, comprising administering to an individual in need thereof a therapeutically effective amount of an agonist antibody that binds to a TREM2 protein.

2. The method of claim 1, wherein the antibody promotes remyelination in one or more demyelination lesions in the CNS of the individual.

3. A method for promoting remyelination of one or more demyelination lesions in an individual having a central nervous system (CNS) demyelination disease, comprising administering to the individual a therapeutically effective amount of an agonist antibody that binds to a TREM2 protein.

4. The method of any one of claims 1-3 wherein the TREM2 protein is a mammalian protein or a human protein.

5. The method of claim 4, wherein the TREM2 protein is a wild-type protein, a naturally occurring variant, or a disease variant.

6. The method of any one of claims 1-5, wherein the antibody induces one or more TREM2 activities selected from the group consisting of:
a. TREM2 binding to DAP12;
b. DAP12 phosphorylation;
c. activation of Syk kinase;
d. recruitment of Syk to a DAP12/TREM2 complex; and
e. increasing activity of one or more TREM2-dependent genes, optionally wherein the one or more TREM2-dependent genes comprise nuclear factor of activated T-cells (NFAT) transcription factors.

7. The method of any one of claims 1-6, wherein the antibody enhances one or more TREM2 activities induced in the presence of myelin.

8. The method of claim 7, wherein the one or more TREM2 activities induced in the presence of myelin comprise increasing activity of one or more TREM2-dependent genes, optionally wherein the one or more TREM2-dependent genes comprise nuclear factor of activated T-cells (NFAT) transcription factors.

9. The method of any one of claims 1-8, wherein the antibody promotes recruitment of oligodendrocyte precursor cells (OPCs) to one or more demyelination lesions in the CNS of the individual.

10. The method of claim 9, wherein the OPCs are PDGFRα positive.

11. The method of any one of claims 1-10, wherein the antibody promotes an increase of mature oligodendrocytes (OLs) in one or more demyelination lesions in the CNS of the individual.

12. The method of any one of claims 1-11, wherein the antibody promotes differentiation of OPCs into mature oligodendrocytes (OLs) in one or more demyelination lesions in the CNS of the individual.

13. The method of claim 11 or claim 12, wherein the mature OLs are OLIG2 and/or CNPase positive.

14. The method of any one of claims 1-13, wherein the antibody promotes an increase in the levels of phosphorylated neurofilaments in one or more demyelination lesions in the CNS of the individual.

15. The method of claim 14, wherein the phosphorylated neurofilaments are SMI-31 positive.

16. The method of any one of claims 1-15, wherein the antibody promotes clearance of myelin debris in one or more demyelination lesions in the individual.

17. The method of any one of claims 1-16, wherein the antibody is a murine antibody, a humanized antibody, a bispecific antibody, a multivalent antibody, a conjugated antibody, or a chimeric antibody.

18. The method of any one of claims 1-17, wherein the antibody is a monoclonal antibody.

19. The method of any one of claims 1-18, wherein the antibody binds to one or more amino acids within amino acid residues 124-153 of SEQ ID NO: 1, or amino acid residues on a TREM2 protein corresponding to amino acid residues 124-153 of SEQ ID NO: 1; within amino acid residues 129-153 of SEQ ID NO: 1, or amino acid residues on a TREM2 protein corresponding to amino acid residues 129-153 of SEQ ID NO: 1; within amino acid residues 140-149 of SEQ ID NO: 1, or amino acid residues on a TREM2 protein corresponding to amino acid residues 140-149 of SEQ ID NO: 1; within amino acid residues 149-157 of SEQ ID NO: 1, or amino acid residues on a TREM2 protein corresponding to amino acid residues 149-157 of SEQ ID NO: 1; or within amino acid residues 153-162 of SEQ ID NO: 1, or amino acid residues on a TREM2 protein corresponding to amino acid residues 153-162 of SEQ ID NO: 1.

20. The method of any one of claims 1-18, wherein the antibody binds to one or more amino acid residues selected from the group consisting of D140, L141, W142, F143, P144, E151, D152, H154, E156, and H157 of SEQ ID NO: 1, or one or more amino acid residues on a mammalian TREM2 protein corresponding to an amino acid residue selected from the group consisting of D140, L141, W142, F143, P144, E151, D152, H154, E156, and H157 of SEQ ID NO: 1.

21. The method of any one of claims 1-20, wherein the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSQWMN (SEQ ID NO: 34), the HVR-H2 comprises the amino acid sequence RIYPGGGDTNYAGKFQG (SEQ ID NO: 35), the HVR-H3 comprises the amino acid sequence ARLLRNQPGESYAMDY (SEQ ID NO: 31), the HVR-L1 comprises the amino acid sequence RSSQSLVHSNRYTYLH (SEQ ID NO: 41), the HVR-L2 comprises the amino acid sequence KVSNRFS (SEQ ID NO: 33), and the HVR-L3 comprises the amino acid sequence SQSTRVPYT (SEQ ID NO: 32).

22. The method of any one of claims 1-21, wherein the antibody comprises a heavy chain variable region comprising the amino acid sequence of SEQ ID NO: 27 and a light chain variable region comprising the amino acid sequence of SEQ ID NO: 30.

23. The method of any one of claims 1-20, wherein the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSDWMN (SEQ ID NO: 36), the HVR-H2 comprises the amino acid sequence RIYPGEGDTNYARKFHG (SEQ ID NO: 37), the HVR-H3 comprises the amino acid sequence ARLLRNKPGESYAMDY (SEQ ID NO: 38), the HVR-L1 comprises the amino acid sequence RTSQSLVHSNAYTYLH (SEQ ID NO: 39), the HVR-L2 comprises the amino acid sequence KVSNRVS (SEQ ID NO: 40), and the HVR-L3 comprises the amino acid sequence SQSTRVPYT (SEQ ID NO: 32).

24. The method of any one of claims 1-20 or 23, wherein the antibody comprises a heavy chain variable region comprising the amino acid sequence of SEQ ID NO: 28 and a light chain variable region comprising the amino acid sequence of SEQ ID NO: 29.

25. The method of any one of claims 1-24, wherein the antibody is a fragment and the fragment is a Fab, Fab′, Fab′-SH, F(ab′)2, Fv or scFv fragment.

26. The method of any one of claims 1-24, wherein the antibody is of the IgG class, the IgM class, or the IgA class.

27. The method of claim 26, wherein the antibody is of the IgG class and has an IgG1, IgG2, IgG3, or IgG4 isotype.

28. The method of claim 26, wherein the antibody has a human IgG1 isotype and comprises amino acid substitutions in the Fc region at the residue positions P331S and E430G, wherein the numbering of the residues is according to EU numbering.

29. The method of any one of claims 1-22, wherein the antibody comprises:
a. a heavy chain comprising the amino acid sequence of SEQ ID NO: 43, and a light chain comprising the amino acid sequence of SEQ ID NO: 47; or
b. a heavy chain comprising the amino acid sequence of SEQ ID NO: 44, and a light chain comprising the amino acid sequence of SEQ ID NO: 47.

30. The method of any one of claims 1-20 or 23-24, wherein the antibody comprises:
a. a heavy chain comprising the amino acid sequence of SEQ ID NO: 45, and a light chain comprising the amino acid sequence of SEQ ID NO: 48; or
b. a heavy chain comprising the amino acid sequence of SEQ ID NO: 46, and a light chain comprising the amino acid sequence of SEQ ID NO: 48.

31. The method of any one of claims 1-30, wherein the individual is a human.

32. The method of any one of claims 1-31, wherein the individual comprises at least one copy of a functional TREM2 gene.

33. The method of claim 32, wherein the individual is heterozygous for a mutation in a TREM2 gene.

34. The method of claim 32, wherein the individual is homozygous for a mutation in a TREM2 gene.

35. The method of any one of claims 1-34, wherein the individual has or is at risk for a disease characteristic selected from the group consisting of myelin damage, one or more demyelination lesions in the CNS, inflammation in the CNS, one or more plaques in the CNS, loss of myelin sheaths, axonal damage, reduced OPCs, reduced OLs, reduced myelin debris clearance, axonal varicosities, axonal spheroids, gliosis, autofluorescent lipid-laden macrophages, axon destruction, and any combination thereof.

36. The method of claim 35, wherein the individual has or is at risk for having axonal damage and/or one or more demyelination lesions in the CNS.

37. The method of claim 36, wherein the axonal damage and/or the one or more demyelination lesions in the CNS are in the white matter, the gray matter, or the corpus callosum of the CNS.

38. The method of any one of claims 1-37, wherein the individual has or is at risk for having a symptom selected from the group consisting of changes in sensation, pricking, numbness, muscle weakness, clonus, muscle spasms, difficulty in moving, difficulties with coordination, difficulties with balance, problems in speech, problems in swallowing, visual problems, fatigue, acute pain, chronic pain, bladder difficulties, bowel difficulties, cognitive impairment, depression, unstable mood, Uhthoffs phenomenon, Lhermitte's sign, and any combination thereof.

39. The method of any one of claims 1-38, wherein the demyelination disease or disorder is multiple sclerosis.
